SEQUENCE LISTING (55 sequences)

SEQ ID NO: 1 - protein, 20 aa, artificial sequence
  CAR signal peptide
ALPVTALLLPLALLLHAARP

SEQ ID NO: 2 - protein, 20 aa, artificial sequence
  Alternative signal peptide
METDTLLLWVLLLWVPGSTG

SEQ ID NO: 3 - protein, 16 aa, artificial sequence
  Fc&#947;RIII&#945; hinge
GLAVSTISSFFPPGYQ

SEQ ID NO: 4 - protein, 45 aa, artificial sequence
  CD8&#945; hinge
TTTPAPRPPTPAPTIASQPLSLRPEACRPAAGGAVHTRGLDFACD

SEQ ID NO: 5 - protein, 231 aa, artificial sequence
  IgG1 hinge
EPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 24 aa, artificial sequence
  CD8&#945; transmembrane domain
IYIWAPLAGTCGVLLLSLVITLYC

SEQ ID NO: 7 - protein, 27 aa, artificial sequence
  4-1BB transmembrane domain
IISFFLALTSTALLFLLFFLTLRFSVV

SEQ ID NO: 8 - protein, 42 aa, artificial sequence
  4-1BB stimulatory domain
KRGRKKLLYIFKQPFMRPVQTTQEEDGCSCRFPEEEEGGCEL

SEQ ID NO: 9 - protein, 112 aa, artificial
  CD3&#950; signalling domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - protein, 15 aa, artificial sequence
  Linker
GGGGSGGGGSGGGGS

SEQ ID NO: 11 - protein, 127 aa, artificial sequence
  CD123 VH
EVKLVESGGGLVQPGGSLSLSCAASGFTFTDYYMSWVRQPPGKALEWLALIRSKADGYTTEYSASVKGRFTLSRDDSQSILYLQMNALRPEDSATYYCARDAAYYSYYSPEGAMDYWGQGTSVTVSS

SEQ ID NO: 12 - protein, 113 aa, artificial sequence
  CD123 VL
MADYKDIVMTQSHKFMSTSVGDRVNITCKASQNVDSAVAWYQQKPGQSPKALIYSASYRYSGVPDRFTGRGSGTDFTLTISSVQAEDLAVYYCQQYYSTPWTFGGGTKLEIKR

SEQ ID NO: 13 - protein, 499 aa, artificial sequence
  123CAR
MALPVTALLLPLALLLHAARPEVKLVESGGGLVQPGGSLSLSCAASGFTFTDYYMSWVRQPPGKALEWLALIRSKADGYTTEYSASVKGRFTLSRDDSQSILYLQMNALRPEDSATYYCARDAAYYSYYSPEGAMDYWGQGTSVTVSSGGGGSGGGGSGGGGSMADYKDIVMTQSHKFMSTSVGDRVNITCKASQNVDSAVAWYQQKPGQSPKALIYSASYRYSGVPDRFTGRGSGTDFTLTISSVQAEDLAVYYCQQYYSTPWTFGGGTKLEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 120 aa, artificial sequence
  CS1 VH
QVQLQQPGAELVRPGASVKLSCKASGYSFTTYWMNWVKQRPGQGLEWIGMIHPSDSETRLNQKFKDKATLTVDKSSSTAYMQLSSPTSEDSAVYYCARSTMIATRAMDYWGQGTSVTVSS

SEQ ID NO: 15 - protein, 107 aa, artificial sequence
  CS1 VL
DIVMTQSQKSMSTSVGDRVSITCKASQDVITGVAWYQQKPGQSPKLLIYSASYRYTGVPDRFTGSGSGTDFTFTISNVQAEDLAVYYCQQHYSTPLTFGAGTKLELK

SEQ ID NO: 16 - protein, 486 aa, artificial sequence
  CS1CAR
MALPVTALLLPLALLLHAARPQVQLQQPGAELVRPGASVKLSCKASGYSFTTYWMNWVKQRPGQGLEWIGMIHPSDSETRLNQKFKDKATLTVDKSSSTAYMQLSSPTSEDSAVYYCARSTMIATRAMDYWGQGTSVTVSSGGGGSGGGGSGGGGSDIVMTQSQKSMSTSVGDRVSITCKASQDVITGVAWYQQKPGQSPKLLIYSASYRYTGVPDRFTGSGSGTDFTFTISNVQAEDLAVYYCQQHYSTPLTFGAGTKLELKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - protein, 120 aa, artificial sequence
  CLL1 VH
EVQLQQSGPELVKPGASVKMSCKASGYTFTSYFIHWVKQKPGQGLEWIGFINPYNDGSKYNEKFKGKATLTSDKSSSTAYMELSSLTSEDSAVYYCTRDDGYYGYAMDYWGQGTSVTVSS

SEQ ID NO: 18 - protein, 107 aa, artificial sequence
  CLL1 VL
DIQMTQSPSSLSASLGERVSLTCRATQELSGYLSWLQQKPDGTIKRLIYAASTLDSGVPKRFSGNRSGSDYSLTISSLESEDFADYYCLQYAIYPYTFGGGTKLEIK

SEQ ID NO: 19 - protein, 487 aa, artificial sequence
  CLL1CAR
MALPVTALLLPLALLLHAARPEVQLQQSGPELVKPGASVKMSCKASGYTFTSYFIHWVKQKPGQGLEWIGFINPYNDGSKYNEKFKGKATLTSDKSSSTAYMELSSLTSEDSAVYYCTRDDGYYGYAMDYWGQGTSVTVSSGGGGSGGGGSGGGGSDIQMTQSPSSLSASLGERVSLTCRATQELSGYLSWLQQKPDGTIKRLIYAASTLDSGVPKRFSGNRSGSDYSLTISSLESEDFADYYCLQYAIYPYTFGGGTKLEIKSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 20 - protein, 124 aa, artificial sequence
  CD22 VH
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSS

SEQ ID NO: 21 - protein, 107 aa, artificial sequence
  CD22 VL
DIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 22 - protein, 490 aa, artificial sequence
  CD22CAR
MALPVTALLLPLALLLHAARPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - protein, 925 aa, artificial sequence
  TRAC_T01-L
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 24 - protein, 925 aa, artificial sequence
  TRAC_T01-R
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPQQVVAIASNNGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 25 - protein, 925 aa, artificial sequence
  CD52_T01-L
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 26 - protein, 925 aa, artificial sequence
  CD52_T01-R
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 27 - protein, 925 aa, artificial sequence
  B2M_T01-L4
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 28 - protein, 925 aa, artificial sequence
  B2M_T01-R4
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 29 - protein, 902 aa, artificial sequence
  CS1_T01-L2
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNIGGKQALETVQALLPVLCQAHGLTPQQVVAIASNGGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPQQVVAIASNGGGRPALESIVAQLSRPDPSGSGSGGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 30 - protein, 902 aa, artificial sequence
  CS1_T01-R2
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPQQVVAIASNNGGKQALETVQRLLPVLCQAHGLTPEQVVAIASNIGGKQALETVQALLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPEQVVAIASNIGGKQALETVQALLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNNGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGRPALESIVAQLSRPDPSGSGSGGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 31 - protein, 22 aa, artificial sequence
  Signal Peptide IC engager (CLL1)
MVLLVTSLLLCELPHPAFLLIP

SEQ ID NO: 32 - protein, 20 aa, artificial sequence
  Signal Peptide IC engager (Trop2
MYRMQLLSCIALSLALVTNS

SEQ ID NO: 33 - protein, 18 aa, artificial sequence
  T2A
EGRGSLLTCGDVEENPGP

SEQ ID NO: 34 - protein, 22 aa, artificial sequence
  P2A
GSGATNFSLLKQAGDVEENPGP

SEQ ID NO: 35 - protein, 239 aa, artificial sequence
  IC engager CD3 scFv
EVQLVESGGGLVQPGKSLKLSCEASGFTFSGYGMHWVRQAPGRGLESVAYITSSSINIKYADAVKGRFTVSRDNAKNLLFLQMNILKSEDTAMYYCARFDWDKNYWGQGTMVTVSSGGGGSGGGGSGGGGSDIQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGPGTKLEIKR

SEQ ID NO: 36 - protein, 240 aa, artificial sequence
  IC engager CD16 scFv
MEVQLVESGGGVVRPGGSLRLSCAASGFTFDDYGMSWVRQAPGKGLEWVSGINWNGGSTGYADSVKGRFTISRDNAKNSLYLQMNSLRAEDTAVYYCARGRSLLFDYWGQGTLVTVSRGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVL

SEQ ID NO: 37 - protein, 490 aa, artificial sequence
  IC engagers TRop2 ScFv
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKGGGGSGGGGSGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLEK

SEQ ID NO: 38 - protein, 487 aa, artificial sequence
  CLL1-CD3 BiTE
EVQLQQSGPELVKPGASVKMSCKASGYTFTSYFIHWVKQKPGQGLEWIGFINPYNDGSKYNEKFKGKATLTSDKSSSTAYMELSSLTSEDSAVYYCTRDDGYYGYAMDYWGQGTSVTVSSGGGGSGGGGSGGGGSDIQMTQSPSSLSASLGERVSLTCRATQELSGYLSWLQQKPDGTIKRLIYAASTLDSGVPKRFSGNRSGSDYSLTISSLESEDFADYYCLQYAIYPYTFGGGTKLEIKGGGGGSEVQLVESGGGLVQPGKSLKLSCEASGFTFSGYGMHWVRQAPGRGLESVAYITSSSINIKYADAVKGRFTVSRDNAKNLLFLQMNILKSEDTAMYYCARFDWDKNYWGQGTMVTVSSGGGGSGGGGSGGGGSDIQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGPGTKLEIKR

SEQ ID NO: 39 - protein, 1094 aa, artificial sequence
  CD123CAR-CLL1BiTE (pCLS35091)
SGEGRGSLLTCGDVEENPGPESALPVTALLLPLALLLHAARPEVKLVESGGGLVQPGGSLSLSCAASGFTFTDYYMSWVRQPPGKALEWLALIRSKADGYTTEYSASVKGRFTLSRDDSQSILYLQMNALRPEDSATYYCARDAAYYSYYSPEGAMDYWGQGTSVTVSSGGGESAGGGSGGGGSMADYKDIVMTQSHKFMSTSVGDRVNITCKASQNVDSAVAWYQQKPGQSPKALIYSASYRYSGVPDRFTGRGSGTDFTLTISSVQAEDLAVYYCQQYYSTPWTFGGGTKLEIKRGSGGGGSCPYSNPSLCSGGGGSCPYSNPSLCSGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMVLLVTSLLLCELPHPAFLLIPEVQLQQSGPELVKPGASVKMSCKASGYTFTSYFIHWVKQKPGQGLEWIGFINPYNDGSKYNEKFKGKATLTSDKSSSTAYMELSSLTSEDSAVYYCTRDDGYYGYAMDYWGQGTSVTVSSGGGGSGGGGSGGGGSDIQMTQSPSSLSASLGERVSLTCRATQELSGYLSWLQQKPDGTIKRLIYAASTLDSGVPKRFSGNRSGSDYSLTISSLESEDFADYYCLQYAIYPYTFGGGTKLEIKGGGGGSEVQLVESGGGLVQPGKSLKLSCEASGFTFSGYGMHWVRQAPGRGLESVAYITSSSINIKYADAVKGRFTVSRDNAKNLLFLQMNILKSEDTAMYYCARFDWDKNYWGQGTMVTVSSGGGGSGGGGSGGGGSDIQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGPGTKLEIKRHHHHHH

SEQ ID NO: 40 - protein, 1094 aa, artificial sequence
  CLL1BiTE-CD123CAR pCLS35092
GSGATNFSLLKQAGDVEENPGPMVLLVTSLLLCELPHPAFLLIPEVQLQQSGPELVKPGASVKMSCKASGYTFTSYFIHWVKQKPGQGLEWIGFINPYNDGSKYNEKFKGKATLTSDKSSSTAYMELSSLTSEDSAVYYCTRDDGYYGYAMDYWGQGTSVTVSSGGGGSGGGGSGGGGSDIQMTQSPSSLSASLGERVSLTCRATQELSGYLSWLQQKPDGTIKRLIYAASTLDSGVPKRFSGNRSGSDYSLTISSLESEDFADYYCLQYAIYPYTFGGGTKLEIKGGGGGSEVQLVESGGGLVQPGKSLKLSCEASGFTFSGYGMHWVRQAPGRGLESVAYITSSSINIKYADAVKGRFTVSRDNAKNLLFLQMNILKSEDTAMYYCARFDWDKNYWGQGTMVTVSSGGGGSGGGGSGGGGSDIQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGPGTKLEIKRHHHHHHSGEGRGSLLTCGDVEENPGPESALPVTALLLPLALLLHAARPEVKLVESGGGLVQPGGSLSLSCAASGFTFTDYYMSWVRQPPGKALEWLALIRSKADGYTTEYSASVKGRFTLSRDDSQSILYLQMNALRPEDSATYYCARDAAYYSYYSPEGAMDYWGQGTSVTVSSGGGESAGGGSGGGGSMADYKDIVMTQSHKFMSTSVGDRVNITCKASQNVDSAVAWYQQKPGQSPKALIYSASYRYSGVPDRFTGRGSGTDFTLTISSVQAEDLAVYYCQQYYSTPWTFGGGTKLEIKRGSGGGGSCPYSNPSLCSGGGGSCPYSNPSLCSGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 41 - protein, 251 aa, artificial sequence
  CD19 scFv
EVQLQQSGPELIKPGASVKMSCKASGYTFTSYVMHWVKQKPGQGLEWIGYINPYNDGTKYNEKFKGKATLTSDKSSSTAYMELSSLTSEDSAVYYCARGTYYYGSRVFDYWGQGTTLTVSSGGGGSGGGGSGGGGSDIVMTQAAPSIPVTPGESVSISCRSSKSLLNSNGNTYLYWFLQRPGQSPQLLIYRMSNLASGVPDRFSGSGSGTAFTLRISRVEAEDVGVYYCMQHLEYPFTFGAGTKLELKRAD

SEQ ID NO: 42 - protein, 504 aa, artificial sequence
  CD19-CD3 BiTE
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 43 - protein, 1028 aa, artificial sequence
  CD22CARxCD19BiTE
ALPVTALLLPLALLLHAARPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMVLLVTSLLLCELPHPAFLLIPEVQLQQSGPELIKPGASVKMSCKASGYTFTSYVMHWVKQKPGQGLEWIGYINPYNDGTKYNEKFKGKATLTSDKSSSTAYMELSSLTSEDSAVYYCARGTYYYGSRVFDYWGQGTTLTVSSGGGGSGGGGSGGGGSDIVMTQAAPSIPVTPGESVSISCRSSKSLLNSNGNTYLYWFLQRPGQSPQLLIYRMSNLASGVPDRFSGSGSGTAFTLRISRVEAEDVGVYYCMQHLEYPFTFGAGTKLELKRADGGGGSEVQLVESGGGLVQPGKSLKLSCEASGFTFSGYGMHWVRQAPGRGLESVAYITSSSINIKYADAVKGRFTVSRDNAKNLLFLQMNILKSEDTAMYYCARFDWDKNYWGQGTMVTVSSGGGGSGGGGSGGGGSDIQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGPGTKLEIKR

SEQ ID NO: 44 - protein, 242 aa, artificial sequence
  CD40_scFv1
EVKLVESGGGLVQPGGSLKLSCATSGFTFSDYYMYWVRQTPEKRLEWVAYINSGGGSTYYPDTVKGRFTISRDNAKNTLYLQMSRLKSEDTAMYYCARRGLPFHAMDYWGQGTSVTVSGSTSGSGKPGSGEGSTKDIQMTQTTSSLSASLGDRVTISCSASQGISNYLNWYQQKPDGTVKLLIYYTSILHSGVPSRFSGSGSGTDYSLTIGNLEPEDIATYYCQQFNKLPPTFGGGTKLEIK

SEQ ID NO: 45 - protein, 249 aa, artificial sequence
  CD40_scFv2
EVQLVQSGAEGVKKPGSSVKVSCKASGYTFTSYWMHWVRQAPGQGLEWIGNIDPSNGETHYNQKFDRATLTVDKSTSTAYMEGLSSLRSEDTAVYYCARERIYYSGSTYDGYFDVWGQGTTVTVSSGSTSGSGKPGSGEGSTKDIQLTQSPSPLSASVGDRVTITCSASSSLSYMHWYQQKPGKSPKRWIYDTSKLASGVPSRFSGSGSGTEYTLTISSLQPEDFATYYCQQWSSNPWTFGGGTKVEIK

SEQ ID NO: 46 - DNA, 1800 bp, homo sapiens
  trac target region
tgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaaggagaagagcagcaggcatgagttgaatgaaggaggcagggccgggtcacagggccttctaggccatgagagggtagacagtattctaaggacgccagaaagctgttgatcggcttcaagcaggggagggacacctaatttgcttttcttttttttttttttttttttttttttttttgagatggagttttgctcttgttgcccaggctggagtgcaatggtgcatcttggctcactgcaacctccgcctcccaggttcaagtgattctcctgcctcagcctcccgagtagctgagattacaggcacccgccaccatgcctggctaattttttgtatttttagtagagacagggtt

SEQ ID NO: 47 - DNA, 467 bp, human
  cd52 target region
agacagccctgagatcacctaaaaagctgctaccaagacagccacgaagatcctaccaaaatgaagcgcttcctcttcctcctactcaccatcagcctcctggttatggtacagatacaaactggactctcaggacaaaacgacaccagccaaaccagcagcccctcagcatccagcaacataagcggaggcattttccttttcttcgtggccaatgccataatccacctcttctgcttcagttgaggtgacacgtctcagccttagccctgtgccccctgaaacagctgccaccatcactcgcaagagaatcccctccatctttgggaggggttgatgccagacatcaccaggttgtagaagttgacaggcagtgccatgggggcaacagccaaaataggggggtaatgatgtaggggccaagcagtgcccagctgggggtcaataaagttacccttgtacttgca

SEQ ID NO: 48 - DNA, 14398 bp, human
  b2m target region
ttaaactgctgacaacttaacactgattactcatagaggcactgccatggtggtcttggataagatctggaagaattctctggattaccaggcagagactcttattgtcttcccttattttcccccaggcaaagtctgctttctgtgatcagctgcctggagttgggggagaagtgacacaaacacccctgtgaccaccactgggactgtgcagggtcagacgtgaagccagcatagtactgggtcttgcccaaggcctatggtaaccaccgcctggctactacctatgttcactcaaggccctatggctctaaaatcagcaggtggcaaagccagccaagcttatgtccctctctttagggtggcaagtttcccccagcctggggtgaatccagagatgccttccaggaactggggcctagtcagaaaccttaggaatctacctggtactctattgcactgaggctgaactggcacccaagccacaagacaaagtccttcccacttttccctcccctttccacaagctgaggagtctcttgccatggccacttccaccccaggcctgtggtgggtactgtctggctactgctgatgtatactcaaggcccaagggctctgcagtaagcttgtggtgaacgctgcccagcctgggactcacccttcagggaagcaggctcccctctggcccagagcaggtccaggaatgctatctaagagccaaagcctggaatcagggaccttaagaggccacttggtattctaaccccacagtggccaagtcccctttactctttccacttcttttctcaagcagaaggaatttctccccatagccaccacagctgtaatgtgctgggtcacaccttaagcctgcacgtctctgagtctcacccaaagcccatgatgagtactacctggctacttctgattattcagggccaaagggctctttagtcagctggtgaggaatcctgccaggactggtccttcccttcaaggcagcaggttcccttctggcccagggtgtggctagaaatgtcagcttggagctatggcctggaatgtgggcctcaggactttgcctggtgccctatcctactgcagctgagctggtatccaatgcaagacaaactcatctttgttcttccctctcctcttctcaagcagaggaaagaagtctctctctgagctttgagctgccctgcctggggttgggagaggggtggtgcaagcaatcccttggccatcccagctagtgtctcactaggttgtgtgccccccacattcactggctctgagcccagcacagcaccaggacttacccaggaattgccatccttgtggcctagactgcctttcaagtttatgtagaacctcagagcacttcagcccatggtggggaggcttactggaactcaggttcccactgctgggatgggtgatttgcctctagctagggctggtctaaatgatccctccatgggacctgggtgagttcttcccagtgttgctttgtgctatgactgggcagcactgagttccaatgcaaagtctgaaaatcactgcactcttcctctcccagaagcacacattctctctccatgccaggtggccgctgccggcgaatgggggagagatagcatttagcaattcaagactgtcttaccctcttcagttactctttcagtgatacgaagttaaaaccaggtactgtgatcactcacctgatttttggttcttatgaaggtgcttttttttgtgtggatcattattcaatttggtgttcctgcagggagcaggattagtggaggcgtctattcagccatcttgctccacctcccctagctatgtccttttaaatatttataatcgacatacaatatattacacttaaagtatacaataagttctaaaatataactgaaatcatcatcgaactctagataattaacatatccatcaaccctaaagtttcctcatgtttagatgatctcctttccctctagaaaccactcatctactttctgtcatatagattatagtttgccttttctggagttttatataaacagaatcatatagtatatgttcttttctgtctgacttcttttatttaccatgattatttcatccatgttacatgtatcaacagtttattccttgtttgttgatgatagtatttcattgtatggatatatactccaatgggtttattcatttacctgttgatgtatgttggatcgtttctagttttcagctatcacaaataaaactgctataaacattaaacattaatgtccaagtctttgtatagacatatgctttcattttctcttgggcaaataccttgggtggagtggctggatattatggccagtgtatgtttaactttataaagagctgtcgaagtgttttcctttttcttttttctttttgagatgaagtcttgctctgttgcccaggctggagtgcagtggtgcaatctcggctcattgcaacctctgcctcccaggttcaagcaattctcctgcctcagcctcctgagtagttggggttacagatgcctgccaccatgcctagctaatttttgtatttttagtagagacagggtttcaccatgttggccaggctggtctcaaactcctgatctcaagtgatctgcccgccttgacctcccaaagtgctcagattacaggtgtgagccaccacatccagccccaaattgtttataccattttatattcctaccaggattgtatgagagttccagttcttctaaattcttgccaacatttactactttgaataaatgttcattgtaaaaaaattttaagagggttatataggggaaaaaaaggccgctattaatctgttaattgcactactgggagataaagctattaaactgtcatatttgtggctgggcgtggtaacacatgcttgtgatcctagcactttaggaggctggggaaggaggatcacttgagaccaggagttcgagaccagcctgggcaacacagcaagactctgtctccacaaaaatattaaaaattaaaaaaaaaagaaattgtcatgtttgtagacttcccaaatttgccatcctaggcaaacagtgctcttgtttttaaacaaatagttgtatacataattttccccctcactattcaaaacatgtcataaatggtaagtccaagaaaaatacaggtattcccccccaaagaaaactgtaaaatcgacttttttctatctgtactgttttttattggtttttaaattggttttccaagtgagtaaatcagaatctatctgtaatggattttaaatttagtgtttctctgtgatgtagtaaacaagaaactagaggcaaaaatagccctgtcccttgctaaacttctaaggcacttttctagtacaactcaacactaacatttcaggcctttagtgccttatatgagtttttaaaagggggaaaagggagggagcaagagtgtcttaactcatacatttaggcataacaattattctcatattttagttattgagagggctggtagaaaaactaggtaaataatattaataattatagcgcttattaaacactacagaacacttactatgtaccaggcattgtgggaggctctctcttgtgcattatctcatttcattaggtccatggagagtattgcattttcttagtttaggcatggcctccacaataaagattatcaaaagcctaaaaatatgtaaaagaaacctagaagttatttgttgtgctccttggggaagctaggcaaatcctttcaactgaaaaccatggtgacttccaagatctctgcccctccccatcgccatggtccacttcctcttctcactgttcctcttagaaaagatctgtggactccaccaccacgaaatggcggcaccttatttatggtcactttagagggtaggttttcttaatgggtctgcctgtcatgtttaacgtccttggctgggtccaaggcagatgcagtccaaactctcactaaaattgccgagccctttgtcttccagtgtctaaaatattaatgtcaatggaatcaggccagagtttgaattctagtctcttagcctttgtttcccctgtccataaaatgaatgggggtaattctttcctcctacagtttatttatatattcactaattcattcattcatccatccattcgttcattcggtttactgagtacctactatgtgccagcccctgttctagggtggaaactaagagaatgatgtacctagagggcgctggaagctctaaagccctagcagttactgcttttactattagtggtcgtttttttctcccccccgccccccgacaaatcaacagaacaaagaaaattacctaaacagcaaggacatagggaggaacttcttggcacagaactttccaaacactttttcctgaagggatacaagaagcaagaaaggtactctttcactaggaccttctctgagctgtcctcaggatgcttttgggactatttttcttacccagagaatggagaaaccctgcagggaattcccaagctgtagttataaacagaagttctccttctgctaggtagcattcaaagatcttaatcttctgggtttccgttttctcgaatgaaaaatgcaggtccgagcagttaactggctggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctgacagcattcgggccgagatgtctcgctccgtggccttagctgtgctcgcgctactctctctttctggcctggaggctatccagcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaagtttagggcgtcgataagcgtcagagcgccgaggttgggggagggtttctcttccgctctttcgcggggcctctggctcccccagcgcagctggagtgggggacgggtaggctcgtcccaaaggcgcggcgctgaggtttgtgaacgcgtggaggggcgcttggggtctgggggaggcgtcgcccgggtaagcctgtctgctgcggctctgcttcccttagactggagagctgtggacttcgtctaggcgcccgctaagttcgcatgtcctagcacctctgggtctatgtggggccacaccgtggggaggaaacagcacgcgacgtttgtagaatgcttggctgtgatacaaagcggtttcgaataattaacttatttgttcccatcacatgtcacttttaaaaaattataagaactacccgttattgacatctttctgtgtgccaaggactttatgtgctttgcgtcatttaattttgaaaacagttatcttccgccatagataactactatggttatcttctgcctctcacagatgaagaaactaaggcaccgagattttaagaaacttaattacacaggggataaatggcagcaatcgagattgaagtcaagcctaaccagggcttttgcgggagcgcatgccttttggctgtaattcgtgcatttttttttaagaaaaacgcctgccttctgcgtgagattctccagagcaaactgggcggcatgggccctgtggtcttttcgtacagagggcttcctctttggctctttgcctggttgtttccaagatgtactgtgcctcttactttcggttttgaaaacatgagggggttgggcgtggtagcttacgcctgtaatcccagcacttagggaggccgaggcgggaggatggcttgaggtccgtagttgagaccagcctggccaacatggtgaagcctggtctctacaaaaaataataacaaaaattagccgggtgtggtggctcgtgcctgtggtcccagctgctccggtggctgaggcgggaggatctcttgagcttaggcttttgagctatcatggcgccagtgcactccagcgtgggcaacagagcgagaccctgtctctcaaaaaagaaaaaaaaaaaaaaagaaagagaaaagaaaagaaagaaagaagtgaaggtttgtcagtcaggggagctgtaaaaccattaataaagataatccaagatggttaccaagactgttgaggacgccagagatcttgagcactttctaagtacctggcaatacactaagcgcgctcaccttttcctctggcaaaacatgatcgaaagcagaatgttttgatcatgagaaaattgcatttaatttgaatacaatttatttacaacataaaggataatgtatatatcaccaccattactggtatttgctggttatgttagatgtcattttaaaaaataacaatctgatatttaaaaaaaaatcttattttgaaaatttccaaagtaatacatgccatgcatagaccatttctggaagataccacaagaaacatgtaatgatgattgcctctgaaggtctattttcctcctctgacctgtgtgtgggttttgtttttgttttactgtgggcataaattaatttttcagttaagttttggaagcttaaataactctccaaaagtcataaagccagtaactggttgagcccaaattcaaacccagcctgtctgatacttgtcctcttcttagaaaagattacagtgatgctctcacaaaatcttgccgccttccctcaaacagagagttccaggcaggatgaatctgtgctctgatccctgaggcatttaatatgttcttattattagaagctcagatgcaaagagctctcttagcttttaatgttatgaaaaaaatcaggtcttcattagattccccaatccacctcttgatggggctagtagcctttccttaatgatagggtgtttctagagagatatatctggtcaaggtggcctggtactcctccttctccccacagcctcccagacaaggaggagtagctgccttttagtgatcatgtaccctgaatataagtgtatttaaaagaattttatacacatatatttagtgtcaatctgtatatttagtagcactaacacttctcttcattttcaatgaaaaatatagagtttataatattttcttcccacttccccatggatggtctagtcatgcctctcattttggaaagtactgtttctgaaacattaggcaatatattcccaacctggctagtttacagcaatcacctgtggatgctaattaaaacgcaaatcccactgtcacatgcattactccatttgatcataatggaaagtatgttctgtcccatttgccatagtcctcacctatccctgttgtattttatcgggtccaactcaaccatttaaggtatttgccagctcttgtatgcatttaggttttgtttctttgttttttagctcatgaaattaggtacaaagtcagagaggggtctggcatataaaacctcagcagaaataaagaggttttgttgtttggtaagaacataccttgggttggttgggcacggtggctcgtgcctgtaatcccaacactttgggaggccaaggcaggctgatcacttgaagttgggagttcaagaccagcctggccaacatggtgaaatcccgtctctactgaaaatacaaaaattaaccaggcatggtggtgtgtgcctgtagtcccaggaatcacttgaacccaggaggcggaggttgcagtgagctgagatctcaccactgcacactgcactccagcctgggcaatggaatgagattccatcccaaaaaataaaaaaataaaaaaataaagaacataccttgggttgatccacttaggaacctcagataataacatctgccacgtatagagcaattgctatgtcccaggcactctactagacacttcatacagtttagaaaatcagatgggtgtagatcaaggcaggagcaggaaccaaaaagaaaggcataaacataagaaaaaaaatggaaggggtggaaacagagtacaataacatgagtaatttgatgggggctattatgaactgagaaatgaactttgaaaagtatcttggggccaaatcatgtagactcttgagtgatgtgttaaggaatgctatgagtgctgagagggcatcagaagtccttgagagcctccagagaaaggctcttaaaaatgcagcgcaatctccagtgacagaagatactgctagaaatctgctagaaaaaaaacaaaaaaggcatgtatagaggaattatgagggaaagataccaagtcacggtttattcttcaaaatggaggtggcttgttgggaaggtggaagctcatttggccagagtggaaatggaattgggagaaatcgatgaccaaatgtaaacacttggtgcctgatatagcttgacaccaagttagccccaagtgaaataccctggcaatattaatgtgtcttttcccgatattcctcaggtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtggggtaagtcttacattcttttgtaagctgctgaaagttgtgtatgagtagtcatatcataaagctgctttgatataaaaaaggtctatggccatactaccctgaatgagtcccatcccatctgatataaacaatctgcatattgggattgtcagggaatgttcttaaagatcagattagtggcacctgctgagatactgatgcacagcatggtttctgaaccagtagtttccctgcagttgagcagggagcagcagcagcacttgcacaaatacatatacactcttaacacttcttacctactggcttcctctagcttttgtggcagcttcaggtatatttagcactgaacgaacatctcaagaaggtataggcctttgtttgtaagtcctgctgtcctagcatcctataatcctggacttctccagtactttctggctggattggtatctgaggctagtaggaagggcttgttcctgctgggtagctctaaacaatgtattcatgggtaggaacagcagcctattctgccagccttatttctaaccattttagacatttgttagtacatggtattttaaaagtaaaacttaatgtcttccttttttttctccactgtctttttcatagatcgagacatgtaagcagcatcatggaggtaagtttttgaccttgagaaaatgtttttgtttcactgtcctgaggactatttatagacagctctaacatgataaccctcactatgtggagaacattgacagagtaacattttagcagggaaagaagaatcctacagggtcatgttcccttctcctgtggagtggcatgaagaaggtgtatggccccaggtatggccatattactgaccctctacagagagggcaaaggaactgccagtatggtattgcaggataaaggcaggtggttacccacattacctgcaaggctttgatctttcttctgccatttccacattggacatctctgctgaggagagaaaatgaaccactcttttcctttgtataatgttgttttattcttcagacagaagagaggagttatacagctctgcagacatcccattcctgtatggggactgtgtttgcctcttagaggttcccaggccactagaggagataaagggaaacagattgttataacttgatataatgatactataatagatgtaactacaaggagctccagaagcaagagagagggaggaacttggacttctctgcatctttagttggagtccaaaggcttttcaatgaaattctactgcccagggtacattgatgctgaaaccccattcaaatctcctgttatattctagaacagggaattgatttgggagagcatcaggaaggtggatgatctgcccagtcacactgttagtaaattgtagagccaggacctgaactctaatatagtcatgtgttacttaatgacggggacatgttctgagaaatgcttacacaaacctaggtgttgtagcctactacacgcataggctacatggtatagcctattgctcctagactacaaacctgtacagcctgttactgtactgaatactgtgggcagttgtaacacaatggtaagtatttgtgtatctaaacatagaagttgcagtaaaaatatgctattttaatcttatgagaccactgtcatatatacagtccatcattgaccaaaacatcatatcagcattttttcttctaagattttgggagcaccaaagggatacactaacaggatatactctttataatgggtttggagaactgtctgcagctacttcttttaaaaaggtgatctacacagtagaaattagacaagtttggtaatgagatctgcaatccaaataaaataaattcattgctaacctttttcttttcttttcaggtttgaagatgccgcatttggattggatgaattccaaattctgcttgcttgctttttaatattgatatgcttatacacttacactttatgcacaaaatgtagggttataataatgttaacatggacatgatcttctttataattctactttgagtgctgtctccatgtttgatgtatctgagcaggttgctccacaggtagctctaggagggctggcaacttagaggtggggagcagagaattctcttatccaacatcaacatcttggtcagatttgaactcttcaatctcttgcactcaaagcttgttaagatagttaagcgtgcataagttaacttccaatttacatactctgcttagaatttgggggaaaatttagaaatataattgacaggattattggaaatttgttataatgaatgaaacattttgtcatataagattcatatttacttcttatacatttgataaagtaaggcatggttgtggttaatctggtttatttttgttccacaagttaaataaatcataaaacttgatgtgttatctcttatatctcactcccactattacccctttattttcaaacagggaaacagtcttcaagttccacttggtaaaaaatgtgaaccccttgtatatagagtttggctcacagtgtaaagggcctcagtgattcacattttccagattaggaatctgatgctcaaagaagttaaatggcatagttggggtgacacagctgtctagtgggaggccagccttctatattttagccagcgttctttcctgcgggccaggtcatgaggagtatgcagactctaagagggagcaaaagtatctgaaggatttaatattttagcaaggaatagatatacaatcatcccttggtctccctgggggattggtttcaggaccccttcttggacaccaaatctatggatatttaagtcccttctataaaatggtatagtatttgcatataacctatccacatcctcctgtatactttaaatcatttctagattacttgtaatacctaatacaatgtaaatgctatgcaaatagttgttattgtttaaggaataatgacaagaaaaaaaagtctgtacatgctcagtaaagacacaaccatccctttttttccccagtgtttttgatccatggtttgctgaatccacagatgtggagcccctggatacggaaggcccgctgtactttgaatgacaaataacagatttaaaattttcaaggcatagttttatacctgatggccagctttgtttatttgaccaagaatctgagttagctagttctaggtactgacaggataaataaaacacaacactgctcccgatcttctcagtttagcagagggacagatatgcactcaaataattaaaatatatcctgataagaatatagcataggtacgcgcgaagaacttggcaatcgaaattttgttgttcaggctgggcgaggtgtctcatgtctgtaatcccagcactttgggaggccatggtaggatgatcgcttgagcccaggagtccgagaccagcctgggcaacataacaagaccctgtctcaattcaaagaattgaattaaaaaaaacaaaaaataatttttttaaaaaagaaatgttgttgttcaaggaacaacaacaaaaatctagggaggtgttagagaagccatttgcctgagctgagagtaagttgctagtggttctcttgattggtaggtggggcctgggtttccaggcatggtagccaggaaggacagccacatggcaggtttgggtaattccaaacagtggaggaagggtgtctgggggaagacttgtaggaactcagctgaaaaaattgggggatgatactctgaaagaaaaacaaagttttaaaatttctactcttacacttaacacataatgcttctgtgaccggatatttaggggttttccccccacactctgttaggagaaaaattttagacagattaaatttaacagagtttaactgagcaaaaatgattctcgaaccaggcagctgccggagccagaataggttcaaaatgactctgggggtgccacatggttggatgacatttagggacagaaaaaggaaagtgatgtgcagaaaatggaagtcaggggcagaagcagccagattggttgcagttcagcatttgcctcatttaaacagggtttgaagagttggccacctgtgattggctgagactctgtggtataagagtaagttacagtctgtttacacatccagttaggttacagttcactatgcagagagaaatctttagcctgaacttacacagggaggcagttttatttatttatttaattttttttttttgagacaaggtctcactctgtcacccgggctggagttcagtggtatgatcatggcttattgcagcctcgacttcctggcctcaagcaatccttccgcctcaggctctagagtagctgggactacaggcacatgtcagcatgcctggctaattttgttttttaatttttagtagagatgaactcctggccttgcacaattctttcgcctcagcctccgaaaatgctgggattacaggtgtgagccactgtgcccagctaaggcaactttaggctaaacctttttttgagacagagtttctctcttgttgtctaggctggagtgcagttgcaccatcttggctcactgcaacctccacctccagggttcaagtgattctcgttcctcagcctcccgagtaactgggattacaggcatgcgccaccacgcctggctaattttgtgtttttagtagagatggtgtttcaccatgttgtccaggctggtctcaaactcctggcctcaagtgatcctctggcctcagcttcccagagtactgagattacaggcatgagccactgtgccctgcctaggctaaacttaatttaacaacaccaaacaatctccagcagacaccaactgggtatcccataattcaattcgattttgattggatctacctggagatggtgtcagatcccgctggttgagggttcagtcccacaagactgccctccacttcagatgccaatcacacattgtaggttgttacctctacttctgactgaccagctggaaaccagaactcccatgactgcctccttgactttggttaatttgctaggacagttcatatttaccaatctattataaaagattaaaggctacagacgaataactagatgaaaagatgaatagggctatatg

SEQ ID NO: 49 - DNA, 2707 bp, Homo sapiens
  cs1 target region
gtaatacctaagagggaagtggcttcatttcagtggctgacttccagagagcaatatggctggttccccaacatgcctcaccctcatctatatcctttggcagctcacagggtcagcagcctctggacccgtgaaagagctggtcggttccgttggtggggccgtgactttccccctgaagtccaaagtaaagcaagttgactctattgtctggaccttcaacacaacccctcttgtcaccatacagccagaagggggcactatcatagtgacccaaaatcgtaatagggagagagtagacttcccagatggaggctactccctgaagctcagcaaactgaagaagaatgactcagggatctactatgtggggatatacagctcatcactccagcagccctccacccaggagtacgtgctgcatgtctacgagcacctgtcaaagcctaaagtcaccatgggtctgcagagcaataagaatggcacctgtgtgaccaatctgacatgctgcatggaacatggggaagaggatgtgatttatacctggaaggccctggggcaagcagccaatgagtcccataatgggtccatcctccccatctcctggagatggggagaaagtgatatgaccttcatctgcgttgccaggaaccctgtcagcagaaacttctcaagccccatccttgccaggaagctctgtgaaggtgctgctgatgacccagattcctccatggtcctcctgtgtctcctgttggtgcccctcctgctcagtctctttgtactggggctatttctttggtttctgaagagagagagacaagaagagtacattgaagagaagaagagagtggacatttgtcgggaaactcctaacatatgcccccattctggagagaacacagagtacgacacaatccctcacactaatagaacaatcctaaaggaagatccagcaaatacggtttactccactgtggaaataccgaaaaagatggaaaatccccactcactgctcacgatgccagacacaccaaggctatttgcctatgagaatgttatctagacagcagtgcactcccctaagtctctgctcaaaaaaaaaacaattctcggcccaaagaaaacaatcagaagaattcactgatttgactagaaacatcaaggaagaatgaagaacgttgacttttttccaggataaattatctctgatgcttctttagatttaagagttcataattccatccactgctgagaaatctcctcaaacccagaaggtttaatcacttcatcccaaaaatgggattgtgaatgtcagcaaaccataaaaaaagtgcttagaagtattcctataaaaatgtaaatgcaaggtcacacatattaatgacagcctgttgtattaatgatggctccaggtcagtgtctggagtttcattccatcccagggcttggatgtcaggattataccaagagtcttgctaccaggagggcaagaagaccaaaacagacagacaagtccagcagaagcagatgcacctgacaaaaatggatgtattaattggctctataaactatgtgcccagcactatgctgagcttacactaattggtcagacatgctgtctgccctcatgaaattggctccaaatgaatgaactactttcatgagcagttgtagcaggcctgaccacagattcccagagggccaggtgtggatccacaggacttgaaggtcaaagttcacaaagatgaagaatcagggtagctgaccatgtttggcagatactataatggagacacagaagtgtgcatggcccaaggacaaggacctccagccaggcttcatttatgcacttgtgctgcaaaagaaaagtctaggttttaaggctgtgccagaacccatcccaataaagagaccgagtctgaagtcacattgtaaatctagtgtaggagacttggagtcaggcagtgagactggtggggcacggggggcagtgggtacttgtaaacctttaaagatggttaattcattcaatagatatttattaagaacctatgcggcccggcatggtggctcacacctgtaatcccagcactttgggaggccaaggtgggtgggtcatctgaggtcaggagttcaagaccagcctggccaacatggtgaaaccccatctctactaaagatacaaaaatttgctgagcgtggtggtgtgcacctgtaatcccagctactcgagaggccaaggcatgagaatcgcttgaacctgggaggtggaggttgcagtgagctgagatggcaccactgcactccggcctaggcaacgagagcaaaactccaatacaaacaaacaaacaaacacctgtgctaggtcagtctggcacgtaagatgaacatccctaccaatacagagctcaccatctcttatacttaagtgaaaaacatggggaaggggaaaggggaatggctgcttttgatatgttccctgacacatatcttgaatggagacctccctaccaagtgatgaaagtgttgaaaaacttaataacaaatgcttgttgggcaagaatgggattgaggattatcttctctcagaaaggcattgtgaaggaattgagccagatctctctccctactgcaaaaccctattgtagtaaaaaagtcttctttactatcttaataaaacagatattgtgagattcacatacaaaaa

SEQ ID NO: 50 - DNA, 49 bp, Homo sapiens
  TRAC T01-target
ttgtcccacagatatccagaaccctgaccctgccgtgtaccagctgaga

SEQ ID NO: 51 - DNA, 47 bp, Homo sapiens
  CD52 T01-target
ttagctgtgctcgcgctactctctctttctggcctggaggctatcca

SEQ ID NO: 52 - DNA, 47 bp, Homo sapiens
  B2M T01-target
tatatcctttggcagctcacaggtgagtccggccggattctcttcca

SEQ ID NO: 53 - DNA, 47 bp, Homo sapiens; identical to SEQ ID NO: 52
  CS1 T01-target

SEQ ID NO: 54 - DNA, 45 bp, artificial sequence
  GMCSF_Tal_1
tgtggctgcagagcctgctgctcttgggcactgtggcctgcagca

SEQ ID NO: 55 - DNA, 45 bp, artificial sequence
  GMCSF_Tal_2
tcctgaacctgagtagagacactgctgctgagatggtaagtgaga

(19) United States
(12) Patent Application Publication
(10) Pub. No.: US 2023/0248825 A1
(43) Pub. Date: Aug. 10, 2023

(54) T-CELLS EXPRESSING IMMUNE CELL ENGAGERS IN ALLOGENIC SETTINGS

(71) Applicant: CELLECTIS S.A., PARIS (FR)

(72) Inventors: Shipra DAS, PARIS (FR); Sumin JO, PARIS (FR); Alexandre JUILLERAT, PARIS (FR); Julien VALTON, PARIS (FR); Laurent POIROT, PARIS (FR); Philippe DUCHATEAU, PARIS (FR)

(21) Appl. No.: 18/005,284

(22) PCT Filed: Jul. 23, 2021
(86) PCT No.: PCT/EP2021/070684

§ 371 Date: Jan. 12, 2023

Related U.S. Application Data
(60) Provisional application No. 63/056,293, filed on Jul. 24, 2020.

(30) Foreign Application Priority Data
Nov. 4, 2020 (DK) PA 202070731

(51) Int. Cl. A61K 39/00; C12N 9/22; C12N 15/11; C12N 15/90; C12N 5/0783; C12N 15/113; A61K 39/395; A61P 35/00

(52) U.S. Cl. CPC A61K 39/4633; C12N 9/22; C12N 15/11; C12N 15/907; C12N 5/0636; A61K 39/4611; A61K 39/4643; C12N 15/1138; A61K 39/3955; A61K 39/4631; A61P 35/00; C12N 2800/80; C12N 2310/20; C12N 2506/11; C12N 2501/515; C12N 2501/51; C12N 2501/2302; A61K 2039/5158; C12N 2310/531; C12N 2310/14

(57) ABSTRACT

The invention relates to therapeutic compositions for allogeneic cellular therapy comprising TCR deficient T-cells, which are genetically engineered to express immune cell engagers, and methods related thereto.

### FIELD OF THE INVENTION

The present invention relates to the field of cell immunotherapy and more particularly to allogenic therapeutic approaches, where T-cells originating from donors are engineered to express immune cell engagers in order to increase their persistence and in addition to potentiate their anti-tumoral activity.

### BACKGROUND

Adoptive cell therapy, also known as cellular immunotherapy, is a form of treatment that uses the cells of our immune system to eliminate pathological cells, such as infected or malignant cells. Some of these approaches involve directly isolating our own immune cells and simply expanding their numbers, whereas others involve genetically engineering immune cells from patients (autologous approach) or donors (allogeneic approach) to boost and/or redirect them towards specific target tissues. In the case of cancer, immune cells known as immune cytolytic lymphocytes are particularly powerful against cancer, due to their ability to bind to markers known as antigens on the surface of cancer cells. Cellular immunotherapies take advantage of this natural ability and can be deployed in different ways: Tumor-Infiltrating Lymphocyte (TIL) Therapy, Engineered T Cell Receptor (TCR) Therapy, Chimeric Antigen Receptor (CAR) T Cell Therapy and Natural Killer (NK) Cell Therapy.

Chimeric antigen receptors (“CAR”) expressing immune cells are cells which have been genetically engineered to express chimeric antigen receptors (CARs) usually designed to recognize specific tumor antigens and kill cancer cells that express the tumor antigen. These are generally T cells expressing CARs (“CAR-T cells”) or Natural Killer cells expressing CARs (“CAR-NK cells”) or macrophages expressing CARs.

CARs are synthetic receptors consisting of a targeting moiety that is associated with one or more signalling domains in a single fusion molecule. In general, the binding moiety of a CAR consists of an antigen-binding domain of a single-chain antibody (scFv), comprising the light and heavy variable fragments of a monoclonal antibody joined by a flexible linker. Binding moieties based on receptor or ligand domains have also been used successfully. The signalling domains for first generation CARs are derived from the cytoplasmic region of the ζCD3zeta or the Fc receptor gamma chains. First generation CARs have been shown to successfully redirect T cell cytotoxicity, however, they failed to provide prolonged expansion and anti-tumor activity in vivo. Signalling domains from co-stimulatory molecules including CD28, OX-40 (CD134), ICOS and 4-1BB (CD137) have been added alone (second generation) or in combination (third generation) to enhance survival and increase proliferation of CAR modified T cells. CARs have successfully allowed T cells to be redirected against antigens expressed at the surface of tumor cells from various malignancies including lymphomas and solid tumors (Jena, Dotti et al. 2010, Blood 116(7):1035-44).

Adoptive immunotherapy, which involves the transfer of autologous or allogeneic antigen-specific T cells generated ex vivo, is a promising strategy to treat viral infections and cancer as confirmed by the increase in the number of CAR-T cells clinical trials.

So far, only autologous CAR T-cells have been approved by the US Food and Drug Administration (FDA) (e.g. Novartis' anti-CD19 CAR-T tisagenlecleucel (Kymriah™) for the treatment of precursor B-cell acute lymphoblastic leukemia, Kite Pharma's anti-CD19 CAR-T axicabtagene ciloleucel (Yescarta™) for certain types of large B-cell lymphoma in adult patients expressing CD19 as a marker). Allogeneic approaches are more challenging due to the alloreactivity of the cells with respect to the patient's own immune cells. The most advanced programs consist of inactivating endogenous T-cell receptor genes by using specific rare-cutting endonucleases, in particular TALE-nucleases, to reduce the alloreactivity of the cells prior to administering them to patients as reported by Poirot et al. [Multiplex Genome-Edited T-cell Manufacturing Platform for “Off-the-Shelf” Adoptive T-cell Immunotherapies (2015) Cancer. Res. 75 (18): 3853-3864] and Qasim, W. et al. [Molecular remission of infant B-ALL after infusion of universal TALEN gene-edited CAR T cells. Science Translational 9(374)]. Meanwhile, inactivation of TCR in primary T-cells can be combined with the inactivation of MHC components such as β2m and also further genes encoding checkpoint inhibitor proteins, such as described for instance in WO2014184744.

More recently, Choi et al. [CAR-T cells secreting BiTEs circumvent antigen escape without detectable toxicity (2019) Nature Biotech 37:1049-58] have engineered autologous CAR-T cells to circumvent antigen escape by the expression of bi-specific T-cells engagers (BiTE). These transgenic BiTEs, which are secreted by autologous CAR-T cells bind, on the one hand, to target antigens CD19 or EGFRvIII, and on the other hand, to TCR by targeting CD3 antigen. These BiTEs help bringing together a patient's autologous T-cells with the tumor cells that are either CD19 or EGFRvIII positive, thereby optimizing CAR-T efficiency and limiting antigen escape. However, this approach could not be applied in allogeneic treatment settings where patient's immune cells are generally depleted by a previous lymphodepletion regimen and the allogeneic immune cells are TCR deficient (lack CD3 at the cell surface).

### SUMMARY OF THE INVENTION

The present invention lies, at least in part, on the unexpected finding that immune cell engagers can be expressed in/by TCR deficient T-cells and can be useful in allogeneic T-cell therapy. These immune cell engagers help engineered CAR-T cells in allogeneic adoptive transfer settings by preventing their elimination by the patient's non-engineered immune cells and by redirecting the activity of patient's NK cells, T-cells and other immune cell types toward pathological cells.

The inventors have found, in particular, that expressing immune cell engagers by allogeneic TCR deficient T-cells (1) allows synergistic effects, while minimizing fratricide killing between the allogeneic T-cells and the patient's immune cells, and (2) improves persistence and efficacy of such allogeneic T-cells in patients.

The methods of the invention can comprise knocking out TCR in the allogeneic T cells and transfecting the cells with viral vector(s) to introduce exogenous polynucleotide sequence(s) encoding at least one immune cell engager. As a specific example, an AAV6 vector comprising sequences encoding a CAR and/or an immune cell engager can be inserted at the TCR locus to obtain expression of the CAR, inactivation of the TCR and/or secretion of the immune cell engager(s) in vivo in the tumour environment.

In more specific embodiments, the invention may be combined with the genetic inactivation of a β2m locus and re-expression of HLAE in the allogeneic engineered T-cell (under control or not of the endogenous β2m promoter) to minimize the rejection of the allogenic cells by the patient's immune cells. To minimize any toxicity linked to a constitutive secretion of immune cell engagers by the allogeneic T-cells, the polynucleotide sequences encoding the immune cell engagers can also be integrated at various endogenous loci, which are dependent on T-cell activation, for example regulated by the TCR activation pathway, such as PD1, CD25, TIM3, LAG3, GM-CSF and CD69 as non-limiting examples.

The invention is broadly drawn to engineered therapeutic T-cells in which TCR expression is reduced or inactivated and which artificially express immune cell engager (IC engager) as well as to the methods for obtaining them.

Such methods according to the invention generally comprise:

(i) Providing a population of genetically engineered T-cells originating from a donor, in which expression of T-cell receptor is inhibited or inactivated;

(ii) Expressing in said population of T-cells, at least one exogenous polynucleotide encoding a soluble immune cell engager (IC engager), said immune cell engager being specifically directed against at least one patient's immune cell type.

The invention is thus drawn to engineered T-cells originating from donors that are TCR deficient, which are further engineered to express immune cell engagers, for their use in allogeneic treatments, especially engineered T-cells originating from donors, the genotype(s) of which are: [TCR]^{negative}[IC engager]^{positive}. Non-limiting examples of IC engagers that can be expressed by the allogeneic engineered immune cells are provided in Tables 12, 13 and 14. More specific examples of engineered cells that can be produced according to the present invention are provided in Table 15.

The present invention discloses various therapeutic strategies and compositions to harness the power of a patient's immune cells, in particular NK, T cells and macrophages, by administrating allogeneic T-cells, especially CAR T-cells, expressing IC engagers, while said allogeneic T cells neutralize and redirect the cytotoxic activity of patient's immune cells to malignant cells, as represented for example in FIGS. 2 to 4.

### BRIEF DESCRIPTION OF THE FIGURES

FIG. 1: A. Schematic representation of patient's T-cells lymphodepleted by using anti-CD52 antibody Alemtuzumab. B. Schematic representation of the allogeneic use in the lymphodepleted patient of an engineered T-cell originating from a donor, which is TCR and CD52 deficient (non-alloreactive and Alemtuzumab resistant) and armored with a transgenic CAR directed against CD123 tumour antigen. C. Schematic representation of the patient recovering one's immune cells: the immune cells detect the allogeneic CAR-T cell's and attack them leading to their elimination.

FIG. 2: Schematic representation of the strategy according to the invention to circumvent the elimination of the CAR-T cells from the recovering patient's immune T-cells by expression and secretion by said CAR-T cells of IC engagers that bridge CD3 and the tumor antigen (e.g. CD123), so that T-cells are redirected toward the tumor cell.

FIG. 3: Schematic representation of a strategy similar to that of FIG. 2 in a context where the patient's recovering T cells are redirected to a second tumor antigen (e.g. CLL1) different from that targeted by the allogeneic CAR T-cell (e.g. CD123).

FIG. 4: A. Schematic representation of cancer patient treated with allogeneic CAR T-cell that is TCR and MHC deficient. In such case, patient's NK cells can detect the absence of MHC and are prompt to eliminate the allogeneic CAR-T cells. B. Schematic representation of the strategy according to the invention to circumvent the elimination of the CAR-T cells by the NK cells from the patient, by expression and secretion by said CAR-T cells of IC engagers that bridge CD16 and the tumor antigen (e.g. CD123), so that the NK cells are redirected toward the tumor cell.

FIG. 5: Schematic representation of vectors (including lentiviral vectors) useful to express CAR and IC engagers in allogeneic T-cells. Promoter(s) can be selected among constitutive or inducible ones.

FIG. 6: Schematic representation of integration strategies at different endogenous T-cell's loci sensitive to T-cell activation by the CAR in order to obtain expression of IC engagers with different time frames.

FIG. 7: Schematic representation of “knock-in” strategies to integrate the sequences encoding the IC-engager and CAR at the TCR (A) or PD1 (B) loci. TCR and PD1 alleles can be optionally inactivated by site directed integration.

FIG. 8: Schematic representation of “knock-in” strategies to integrate the sequences encoding the IC-engager and CAR at the CD25 locus. As per the illustrated example, CD25 allele expression can be maintained.

FIG. 9: Schematic representation of integration strategies to integrate the sequences encoding a CD123CAR along with a CLL1-BiTE (as exemplified) or a CLL1-TriKE at the TRAC locus. Both CD123 CAR and CLL1-BiTE (or TriKE) are expressed under the TCR promoter using self-cleaving peptides. Two orientations (i.e. CAR followed by IC engager or IC engager followed by CAR) were tested.

FIG. 10: Schematic representation of integration strategies to introduce sequences of αTROP2-BiTE (A and B) or αTROP2-TrKE (B and C) at either the PD1 or CD25 locus. These constructs also express a truncated LNGFR polypeptide for detection.

FIG. 11: Cytotoxicity results obtained by a 24 h (A and B) or 48 h (C and D) co-incubation of an increase amount of PBMC with 123CAR expressing CCL1-BiTE (in both orientation) on CD123 and CLL1 positive tumor cell line (THP-1, closed circle and triangle) or on CD123 negative and CLL1 positive tumor cell line (U937, open square and diamond).

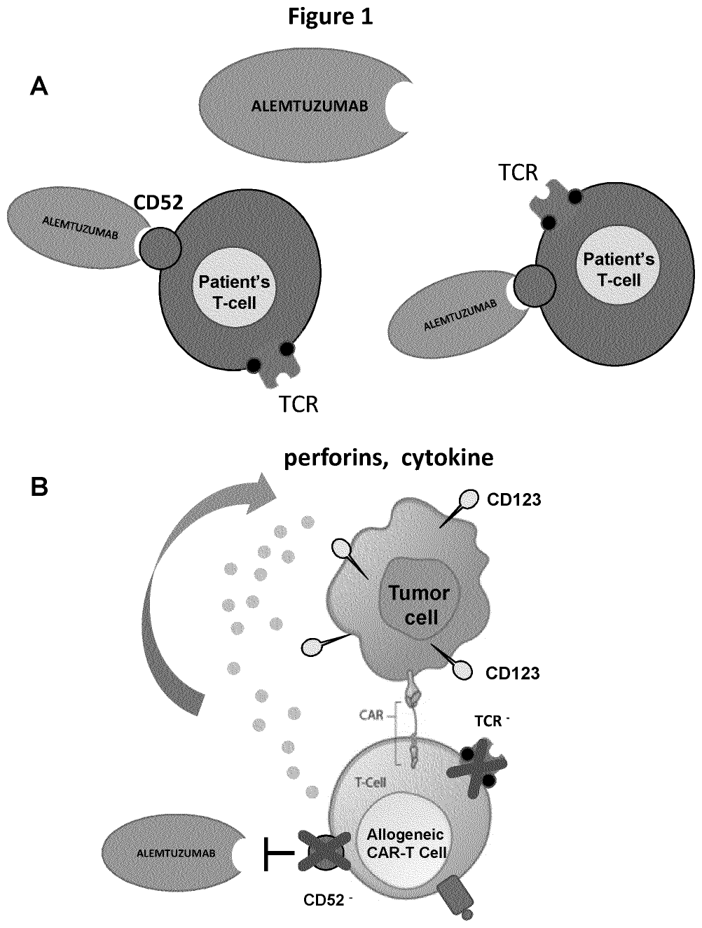

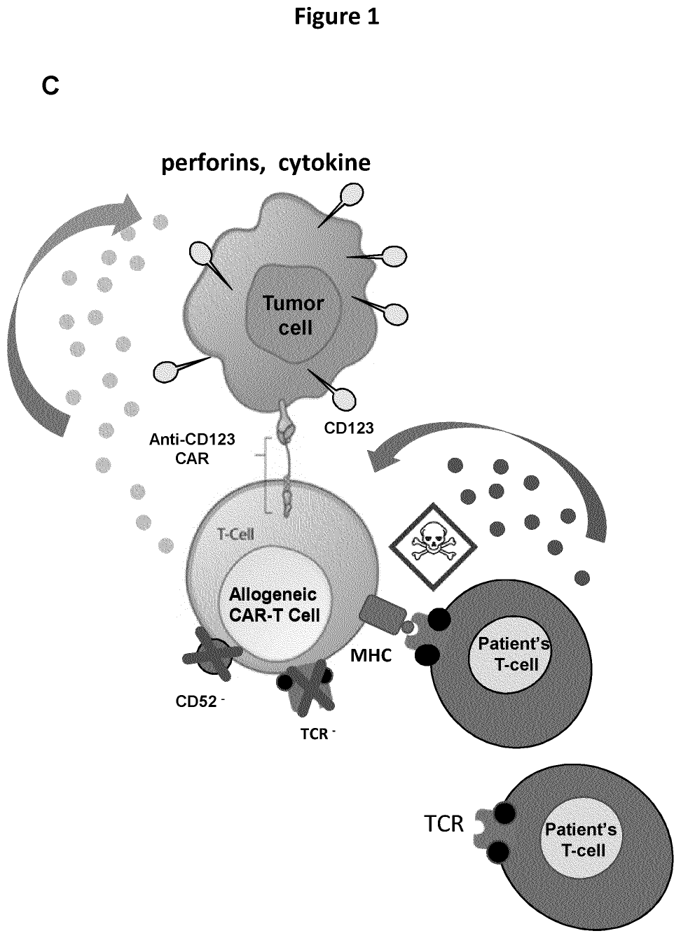

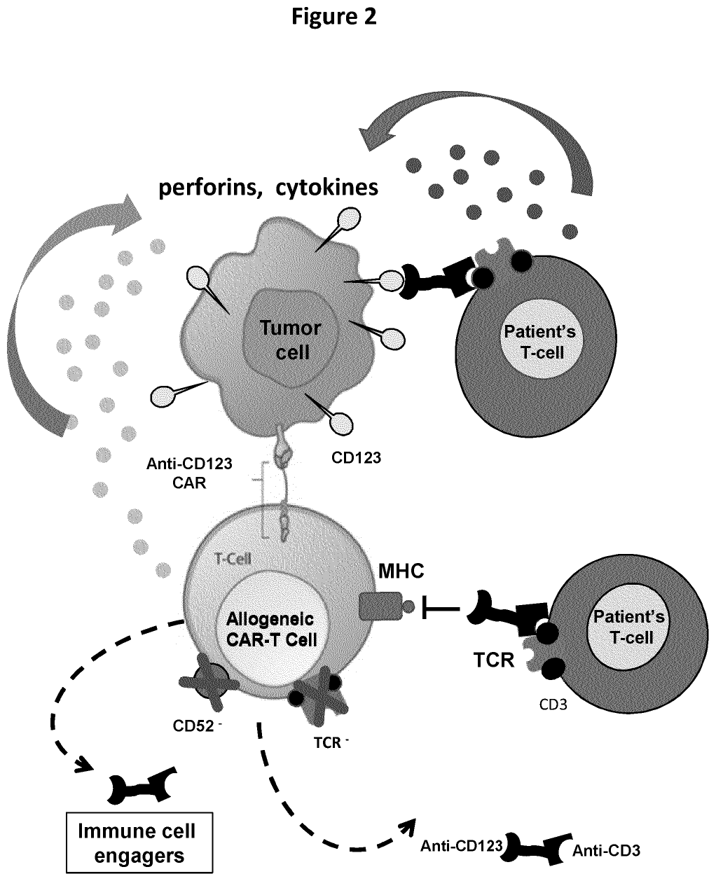

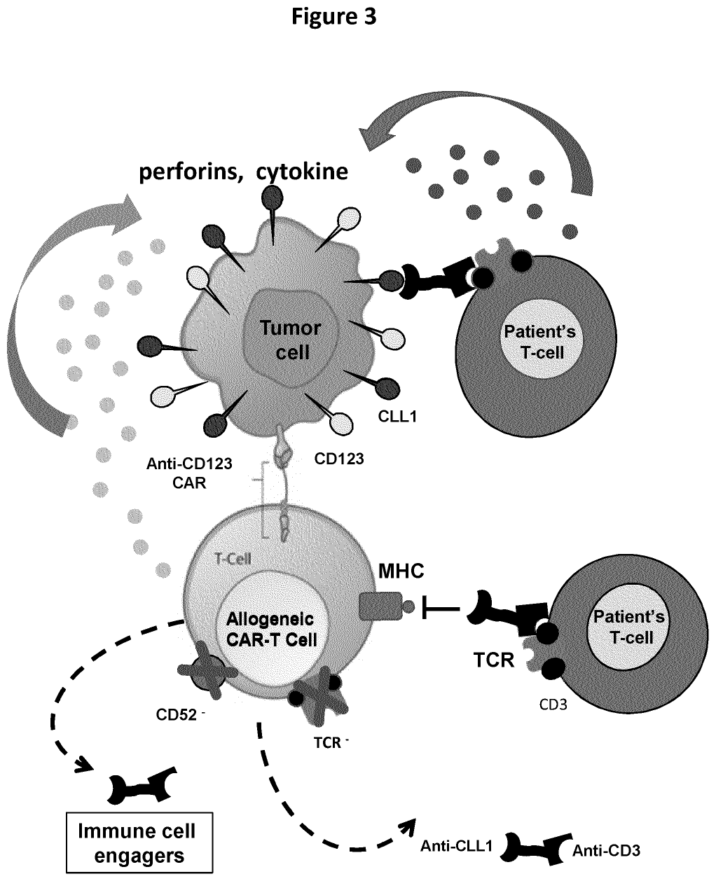

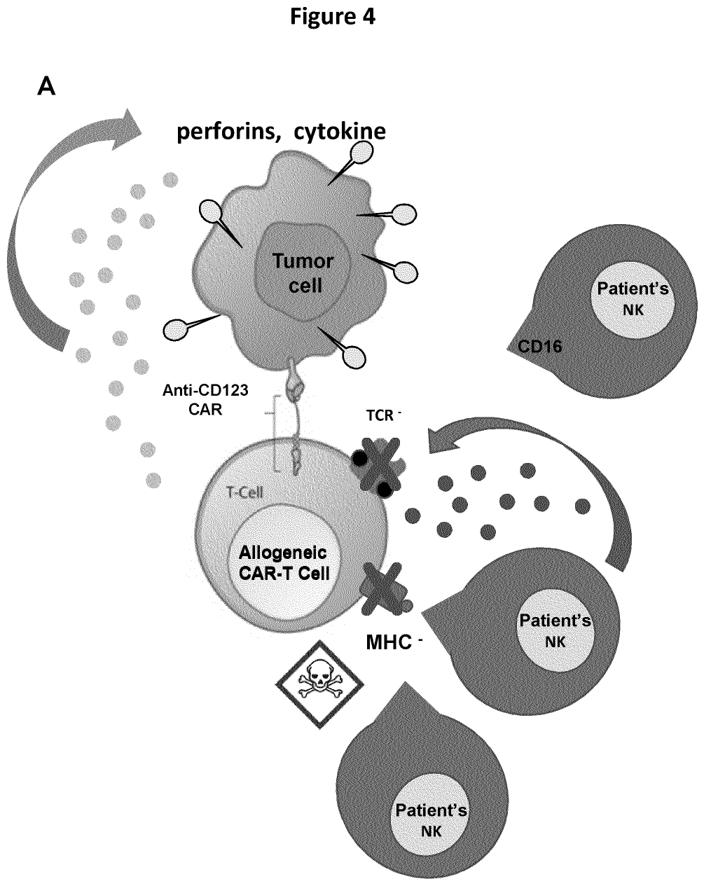

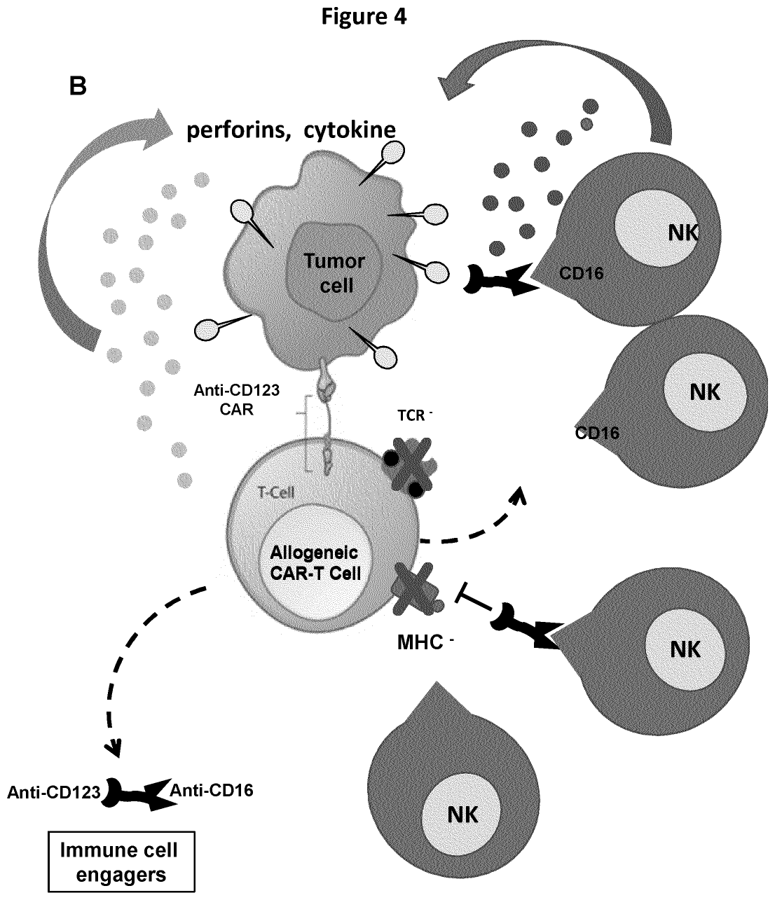

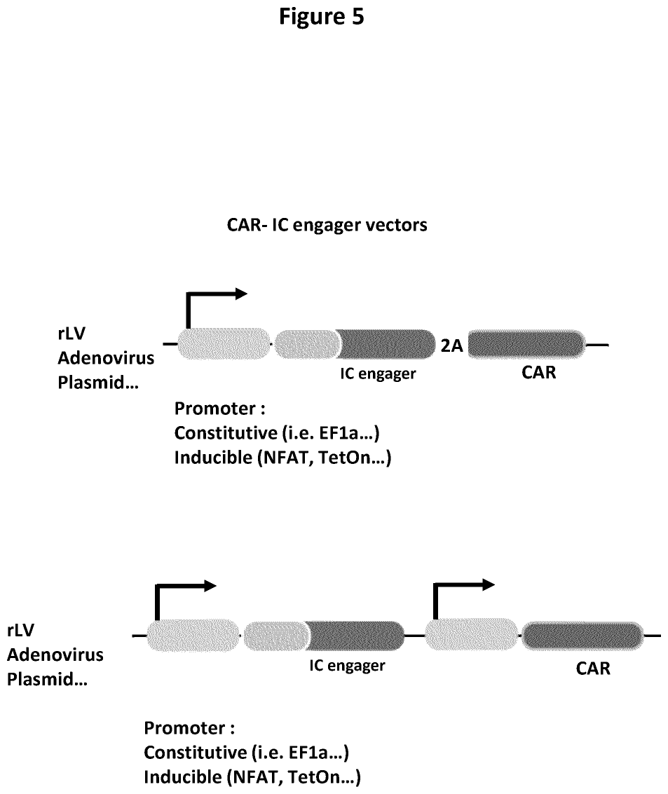

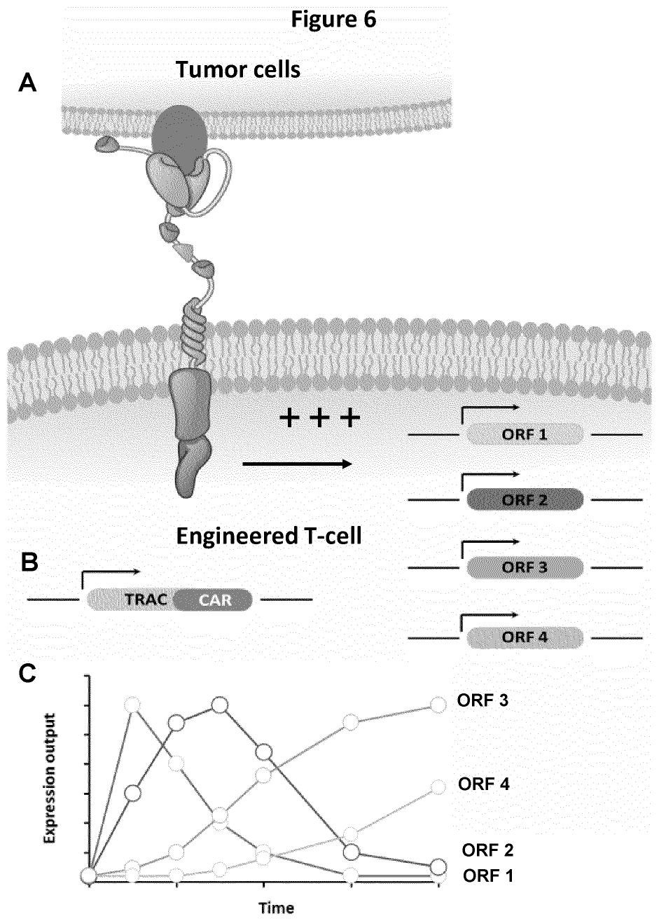

### DETAILED DESCRIPTION

Unless specifically defined herein, all technical and scientific terms used have the same meaning as commonly understood by a skilled artisan in the fields of gene therapy, biochemistry, genetics, and molecular biology.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will prevail. Further, the materials, methods, and examples are illustrative only and are not intended to be limiting, unless otherwise specified.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Current Protocols in Molecular Biology (Frederick M. AUSUBEL, 2000, Wiley and son Inc, Library of Congress, USA); Molecular Cloning: A Laboratory Manual, Third Edition, (Sambrook et al, 2001, Cold Spring Harbor, New York: Cold Spring Harbor Laboratory Press); Oligonucleotide Synthesis (M. J. Gait ed., 1984); Mullis et al. U.S. Pat. No. 4,683,195; Nucleic Acid Hybridization (B. D. Harries & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the series, Methods In ENZYMOLOGY (J. Abelson and M. Simon, eds.-in-chief, Academic Press, Inc., New York), specifically, Vols. 154 and 155 (Wu et al. eds.) and Vol. 185, “Gene Expression Technology” (D. Goeddel, ed.); Gene Transfer Vectors For Mammalian Cells (J. H. Miller and M. P. Calos eds., 1987, Cold Spring Harbor Laboratory); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); and Manipulating the Mouse Embryo, (Cold Spring Harbor Laboratory Press, Cold Spring Harbor, N.Y., 1986).

In one embodiment, the present invention has for its object the use of allogeneic genetically engineered T-cells exogenously expressing soluble immune cell engagers for infusing patients suffering from a cancer or infection.

According to one aspect, the invention pertains to methods for producing therapeutic T-cells, comprising at least one of the following steps:
(i) Providing a population of genetically engineered T-cells originating from a donor, in which expression of T-cell receptor (TCR) is inhibited or inactivated;
(ii) Expressing in said population of T-cells, at least one exogenous polynucleotide encoding a soluble immune cell engager (IC engager), wherein said immune cell engager is specifically directed against at least one patient's immune cell type.
(iii) Optionally, isolating from the population T-cells that do not express TCR alpha at the surface of the cells.

According to preferred aspects, said soluble immune cell engager produced by the engineered T-cells of the invention is specifically directed toward the non-engineered immune cells produced by the patient. Immune cell types are preferably T-cell, NK-cell, macrophage or antigen presenting cells (APC). The immune cell engager preferably binds an immune cell's activating receptor complex of such immune cell type(s) with the effect of activating patient's own immune cells.

According to one aspect, the soluble immune cell engager binds a component of T-cells activating receptor complex (i.e. TCR), such as CD3, TCRalpha, TCRbeta, TCRgamma and/or TCR delta. CD3 is particularly suited as it generally activates patient's T-cells without preventing TCR interactions with the MHC presented by the pathological cells.

According to another aspect, which may parallel the first, the engineered cell can produce an immune cell engager directed against patient's NK cells, especially CD16 surface antigen.

According to another aspect, which does not exclude the previous ones, the soluble immune cell engager can be directed against APC/macrophages, especially CD40 surface antigen.

As mentioned previously, different architectures of soluble immune cell engager are available in the art, such as bispecific t-cell engagers (BITE), dual-affinity re-targeting antibodies (DART), bispecific engagement by antibodies based on the t-cell receptor (BEAT), CROSSMAB, TRIOMAB, tandem diabody (TANDAB), ADAPTIR, affinity-tailored adaptors for t-cells (ATAC), DUOBODY, XMAB, t-cell redirecting antibody (TRAB), BICLONICS, DUTAMAB, VELOCI-BI, hinge-mutated, bispecific antibody-armed activated t-cells (AATC), bi-& tri-specific killer cell engagers (BIKE, TRIKE).

On preferred type of IC engager is a BiTE, such as Blinatumomab (CAS #853426-35-4), which comprises ScFv sequences binding CD3 (ex. SEQ ID NO:35) and CD19, such as for instance SEQ ID NO:42.

According to preferred embodiments, the IC engagers binds at least:
CD3 and CD22;
CD3 and CD20;
CD3 and CD123;
CD3 and CLL1;
CD3 and Mesothelin;
CD3 and Trop2;

According to preferred embodiments, the IC engagers binds at least:
CD16 and CD22;
CD16 and CD20;
CD16 and CD123;
CD16 and CLL1;
CD16 and Mesothelin;
CD16 and Trop2;

According to preferred embodiments, the IC engagers binds at least:
CD40 and CD22;
CD40 and CD20;
CD40 and CD123;
CD40 and CLL1;
CD40 and Mesothelin;
CD40 and Trop2;

The IC engagers expressed in the engineered cells of the present invention preferably comprise polypeptide sequences that have at least 70%, preferably 80%, more preferably 90%, and even more preferably 95 or 99% sequence identity with those referred to in Table 1.

TABLE 1
preferred sequences involved in IC engagers
used in the experimental protocol

| SEQ | IC | |
|---|---|---|
| ID: | engagers | |
| # | sequences | Polypeptide sequences |
| 31 | Signal | MVLLVTSLLLCELPHPAFLLIP |
| | Peptide IC | |
| | Engager | |
| | (CLL1) | |
| 32 | Signal | MYRMQLLSCIALSLALVTNS |
| | Peptide IC | |
| | Engager | |
| | (Trop2 | |
| 33 | T2A | EGRGSLLTCGDVEENPGP |
| 34 | P2A | GSGATNFSLLKQAGDVEENPGP |
| 35 | IC engager | EVQLVESGGGLVQPGKSLKLSCEASGFTFS |
| | CD3 scFv | GYGMHWRQAPGRGLESVAYITSSSINIKYA |
| | | DAVKGRFTVSRDNAKNLLFLQMNILKSEDT |
| | | AMYYCARFDWDKNYWGQGTMVTVSSGGGGS |
| | | GGGGSGGGGSDIQMTQSPSSLPASLGDRVT |
| | | INCQASQDISNYLNWYQQKPGKAPKLLIYY |
| | | TNKLADGVPSRFSGSGSGRDSSFTISSLES |
| | | EDIGSYYCQQYYNYPWTFGPGTKLEIKR |
| 36 | IC engager | MEVQLVESGGGVVRPGGSLRLSCAASGFTF |
| | CD16 scFv | DDYGMSWVRQAPGKGLEWVSGINWNGGSTG |
| | | YADSVKGRFTISRDNAKNSLYLQMNSLRAE |
| | | DTAVYYCARGRSLLFDYWGQGTLVTVSRGG |
| | | GGSGGGGSGGGGSSELTQDPAVSVALGQTV |
| | | RITCQGDSLRSYYASWYQQKPGQAPVLVIY |
| | | GKNNRPSGIPDRFSGSSSGNTASLTITGAQ |
| | | AEDEADYYCNSRDSSGNHVVFGGGTKLTVL |
| 37 | IC engagers | DIQLTQSPSSLSASVGDRVSITCKASQDVS |
| | TRop2 | IAVAWYQQKPGKAPKLLIYSASYRYTGVPD |
| | ScFv | RFSGSGSGTDFTLTISSLQPEDFAVYYCQQ |
| | | HYITPLTFGAGTKVEIKGGGGSGGGGSGGG |
| | | GSQVQLQQSGSELKKPGASVKVSCKASGYT |
| | | FTNYGMNWWKQAPGQGLKWMGWINTYTGEP |
| | | TYTDDFKGRFAFSLDTSVSTAYLQISSLKA |
| | | DDTAVYFCARGGFGSSYWYFDVWGQGSLVT |
| | | VSSGGGGSDIKLQQSGAELARPG |
| | | ASVKMSCKTSGYTFTRYTMHWWKQRPGQGL |
| | | EWIGYINPSRGYTNYNQKFKDKATLTTDKS |
| | | SSTAYMQLSSLTSEDSAVYYCARYYDDHYC |
| | | LDYWGQGTTLTVSSVEGGSGGSGGSGGSGG |
| | | VDDIQLTQSPAIMSASPGEKVTMTCRASSS |
| | | VSYMNWYQQKSGTSPKRWIYDTSKVASGVP |
| | | YRFSGSGSGTSYSLTISSMEAEDAATYYCQ |
| | | QWSSNPLTFGAGTKLEK |
| 38 | CLL1-CD3 | EVQLQQSGPELVKPGASVKMSCKASGYTFT |
| | BITE | SYFIHWKQKPGQGLEWIGFINPYNDGSKYN |
| | | EKFKGKATLTSDKSSSTAYMELSSLTSEDS |
| | | AVYYCTRDDGYYGYAMDYWGQGTSVTVSSG |
| | | GGGSGGGGSGGGGSDIQMTQSPSSLSASLG |
| | | ERVSLTCRATQELSGYLSWLQQKPDGTIKR |
| | | LIYAASTLDSGVPKRFSGNRSGSDYSLTIS |
| | | SLESEDFADYYCLQYAIYPYTFGGGTKLEI |
| | | KGGGGGSEVQLVESGGGLVQPGKSLKLSCE |
| | | ASGFTFSGYGMHWWRQAPGRGLESVAYITS |
| | | SSINIKYADAVKGRFTVSRDNAKNLLFLQM |
| | | NILKSEDTAMYYCARFDWDKNYWGQGTMVT |
| | | VSSGGGGSGGGGSGGGGSDIQMTQSPSSLP |
| | | ASLGDRVTINCQASQDISNYLNWYQQKPGK |
| | | APKLLIYYTNKLADGVPSRFSGSGSGRDSS |
| | | FTISSLESEDIGSYYCQQYYNYPWTFGPGT |
| | | KLEIKR |
| 39 | CD123CAR- | SGEGRGSLLTCGDVEENPGPESALPVTALL |
| | CLL1BITE | LPLALLLHAARPEVKLVESGGGLVQPGGSL |
| | (pCLS35091) | SLSCAASGFTFTDYYMSWWRQPPGKALEWL |
| | | ALIRSKADGYTTEYSASVKGRFTLSRDDSQ |
| | | SILYLQMNALRPEDSATYYCARDAAYYSYY |
| | | SPEGAMDYWGQGTSVTVSSGGGESAGGGSG |
| | | GGGSMADYKDIVMTQSHKFMSTSVGDRVNI |
| | | TCKASQNVDSAVAWYQQKPGQSPKALIYSA |
| | | SYRYSGVPDRFTGRGSGTDFTLTISSVQAE |
| | | DLAVYYCQQYYSTPWTFGGGTKLEIKRGSG |
| | | GGGSCPYSNPSLCSGGGGSCPYSNPSLCSG |
| | | GGGSTTTPAPRPPTPAPTIASQPLSLRPEA |
| | | CRPAAGGAVHTRGLDFACDIYIWAPLAGTC |
| | | GVLLLSLVITLYCKRGRKKLLYIFKQPFMR |
| | | PVQTTQEEDGCSCRFPEEEEGGCELRVKFS |
| | | RSADAPAYQQGQNQLYNELNLGRREEYDVL |
| | | DKRRGRDPEMGGKPRRKNPQEGLYNELQKD |
| | | KMAEAYSEIGMKGERRRGKGHDGLYQGLST |
| | | ATKDTYDALHMQALPPRGSGATNFSLLKQA |
| | | GDVEENPGPMVLLVTSLLLCELPHPAFLLI |
| | | PEVQLQQSGPELVKPGASVKMSCKASGYTF |
| | | TSYFIHWWKQKPGQGLEWIGFINPYNDGSK |
| | | YNEKFKGKATLTSDKSSSTAYMELSSLTSE |
| | | DSAVYYCTRDDGYYGYAMDYWGQGTSVTVS |
| | | SGGGGSGGGGSGGGGSDIQMTQSPSSLSAS |
| | | LGERVSLTCRATQELSGYLSWLQQKPDGTI |
| | | KRLIYAASTLDSGVPKRFSGNRSGSDYSLT |
| | | ISSLESEDFADYYCLQYAIYPYTFGGGTKL |
| | | EIKGGGGGSEVQLVESGGGLVQPGKSLKLS |
| | | CEASGFTFSGYGMHWWRQAPGRGLESVAYI |
| | | TSSSINIKYADAVKGRFTVSRDNAKNLLFL |
| | | QMNILKSEDTAMYYCARFDWDKNYWGQGTM |
| | | VTVSSG |
| 40 | CLL1BITE- | GGGSGGGGSGGGGSDIQMTQSPSSLPASLG |
| | CD123CAR | DRVTINCQASQDISNYLNWYQQKPGKAPKL |
| | pCLS35092 | LIYYTNKLADGVPSRFSGSGSGRDSSFTIS |
| | | SLESEDIGSYYCQQYYNYPWTFGPGTKLEI |
| | | KRHHHHHHGSGATNFSLLKQAGDVEENPGP |
| | | MVLLVTSLLLCELPHPAFLLIPEVQLQQSG |
| | | PELVKPGASVKMSCKASGYTFTSYFIHWWK |
| | | QKPGQGLEWIGFINPYNDGSKYNEKFKGKA |
| | | TLTSDKSSSTAYMELSSLTSEDSAVYYCTR |
| | | DDGYYGYAMDYWGQGTSVTVSSGGGGSGGG |
| | | GSGGGGSDIQMTQSPSSLSASLGERVSLTC |
| | | RATQELSGYLSWLQQKPDGTIKRLIYAAST |
| | | LDSGVPKRFSGNRSGSDYSLTISSLESEDF |
| | | ADYYCLQYAIYPYTFGGGTKLEIKGGGGGS |
| | | EVQLVESGGGLVQPGKSLKLSCEASGFTFS |
| | | GYGMHWRQAPGRGLESVAYITSSSINIKYA |
| | | DAVKGRFTVSRDNAKNLLFLQMNILKSEDT |
| | | AMYYCARFDWDKNYWGQGTMVTVSSGGGGS |
| | | GGGGSGGGGSDIQMTQSPSSLPASLGDRVT |
| | | INCQASQDISNYLNWYQQKPGKAPKLLIYY |
| | | TNKLADGVPSRFSGSGSGRDSSFTISSLES |
| | | EDIGSYYCQQYYNYPWTFGPGTKLEIKRHH |
| | | HHHHSGEGRGSLLTCGDVEENPGPESALPV |
| | | TALLLPLALLLHAARPEVKLVESGGGLVQP |
| | | GGSLSLSCAASGFTFTDYYMSWWRQPPGKA |
| | | LEWLALIRSKADGYTTEYSASVKGRFTLSR |
| | | DDSQSILYLQMNALRPEDSATYYCARDAAY |
| | | YSYYSPEGAMDYWGQGTSVTVSSGGGESAG |
| | | GGSGGGGSMADYKDIVMTQSHKFMSTSVGD |
| | | RVNITCKASQNVDSAVAWYQQKPGQSPKAL |
| | | IYSASYRYSGVPDRFTGRGSGTDFTLTISS |
| | | VQAEDLAVYYCQQYYSTPWTFGGGTKLEIK |
| | | RGSGGGGSCPYSNPSLCSGGGGSCPYSNPS |
| | | LCSGGGGSTTTPAPRPPTPAPTIASQPLSL |
| | | RPEACRPAAGGAVHTRGLDFACDIYIWAPL |
| | | AGTCGVLLLSLVITLYCKRGRKKLLYIFKQ |
| | | PFMRPVQTTQEEDGCSCRFPEEEEGGCELR |
| | | VKFSRSADAPAYQQGQNQLYNELNLGRREE |
| | | YDVLDKRRGRDPEMGGKPRRKNPQEGLYNE |
| | | LQKDKMAEAYSEIGMKGERRRGKGHDGLYQ |
| | | GLSTATKDTYDALHMQALPPR |
| 41 | CD19 scFv | EVQLQQSGPELIKPGASVKMSCKASGYTFT |
| | | SYVMHWWKQKPGQGLEWIGYINPYNDGTKY |
| | | NEKFKGKATLTSDKSSSTAYMELSSLTSED |
| | | SAVYYCARGTYYYGSRVFDYWGQGTTLTVS |
| | | SGGGGSGGGGSGGGGSDIVMTQAAPSIPVT |
| | | PGESVSISCRSSKSLLNSNGNTYLYWFLQR |
| | | PGQSPQLLIYRMSNLASGVPDRFSGSGSGT |
| | | AFTLRISRVEAEDVGVYYCMQHLEYPFTFG |
| | | AGTKLELKRAD |
| 42 | CD19-CD3 | DIQLTQSPASLAVSLGQRATISCKASQSVD |
| | BİTE | YDGDSYLNWYQQIPGQPPKLLIYDASNLVS |
| | | GIPPRFSGSGSGTDFTLNIHPVEKVDAATY |
| | | HCQQSTEDPWTFGGGTKLEIKGGGGSGGGG |
| | | SGGGGSQVQLQQSGAELVRPGSSVKISCKA |
| | | SGYAFSSYWMNWVKQRPGQGLEWIGQIWPG |
| | | DGDTNYNGKFKGKATLTADESSSTAYMQLS |
| | | SLASEDSAVYFCARRETTTVGRYYYAMDYW |
| | | GQGTTVTVSSGGGGSDIKLQQSG |
| 43 | CD22CARx | AELARPGASVKMSCKTSGYTFTRYTMHWWK |
| | CD19BITE | QRPGQGLEWIGYINPSRGYTNYNQKFKDKA |
| | | TLTTDKSSSTAYMQLSSLTSEDSAVYYCAR |
| | | YYDDHYCLDYWGQGTTLTVSSVEGGSGGSG |
| | | GSGGSGGVDDIQLTQSPAIMSASPGEKVTM |
| | | TCRASSSVSYMNWYQQKSGTSPKRWIYDTS |
| | | KVASGVPYRFSGSGSGTSYSLTISSMEAED |
| | | AATYYCQQWSSNPLTFGAGTKLELKHHHHH |
| | | HALPVTALLLPLALLLHAARPQVQLQQSGP |
| | | GLVKPSQTLSLTCAISGDSVSSNSAAWNWI |
| | | RQSPSRGLEWLGRTYYRSKWYNDYAVSVKS |
| | | RITINPDTSKNQFSLQLNSVTPEDTAVYYC |
| | | AREVTGDLEDAFDIWGQGTMVTVSSGGGGS |
| | | GGGGSGGGGSDIQMTQSPSSLSASVGDRVT |
| | | ITCRASQTIWSYLNWYQQRPGKAPNLLIYA |
| | | ASSLQSGVPSRFSGRGSGTDFTLTISSLQA |
| | | EDFATYYCQQSYSIPQTFGQGTKLEIKTTT |
| | | PAPRPPTPAPTIASQPLSLRPEACRPAAGG |
| | | AVHTRGLDFACDIYIWAPLAGTCGVLLLSL |
| | | VITLYCKRGRKKLLYIFKQPFMRPVQTTQE |
| | | EDGCSCRFPEEEEGGCELRVKFSRSADAPA |
| | | YQQGQNQLYNELNLGRREEYDVLDKRRGRD |
| | | PEMGGKPRRKNPQEGLYNELQKDKMAEAYS |
| | | EIGMKGERRRGKGHDGLYQGLSTATKDTYD |
| | | ALHMQALPPRGSGATNFSLLKQAGDVEENP |
| | | GPMVLLVTSLLLCELPHPAFLLIPEVQLQQ |
| | | SGPELIKPGASVKMSCKASGYTFTSYVMHW |
| | | VKQKPGQGLEWIGYINPYNDGTKYNEKFKG |
| | | KATLTSDKSSSTAYMELSSLTSEDSAVYYC |
| | | ARGTYYYGSRVFDYWGQGTTLTVSSGGGGS |
| | | GGGGSGGGGSDIVMTQAAPSIPVTPGESVS |
| | | ISCRSSKSLLNSNGNTYLYWFLQRPGQSPQ |
| | | LLIYRMSNLASGVPDRFSGSGSGTAFTLRI |
| | | SRVEAEDVGVYYCMQHLEYPFTFGAGTKLE |
| | | LKRADGGGGSEVQLVESGGGLVQPGKSLKL |
| | | SCEASGFTFSGYGMHWRQAPGRGLESVAYI |
| | | TSSSINIKYADAVKGRFTVSRDNAKNLLFL |
| | | QMNILKSEDTAMYYCARFDWDKNYWGQGTM |
| | | VTVSSGGGGSGGGGSGGGGSDIQMTQSPSS |
| | | LPASLGDRVTINCQASQDISNYLNWYQQKP |
| | | GKAPKLLIYYTNKLADGVPSRFSGSGSGRD |
| | | SSFTISSLESEDIGSYYCQQYYNYPWTFGP |
| | | GTKLEIKR |
| 44 | CD40_ | EVKLVESGGGLVQPGGSLKLSCATSGFTFS |
| | scFv1 | DYYMYWRQTPEKRLEWAYINSGGGSTYYPD |
| | | TVKGRFTISRDNAKNTLYLQMSRLKSEDTA |
| | | MYYCARRGLPFHAMDYWGQGTSVTVSGSTS |
| | | GSGKPGSGEGSTKDIQMTQTTSSLSASLGD |
| | | RVTISCSASQGISNYLNWYQQKPDGTVKLL |
| | | IYYTSILHSGVPSRFSGSGSGTDYSLTIGN |
| | | LEPEDIATYYCQQFNKLPPTFGGGTKLEIK |
| 45 | CD40_ | EVQLVQSGAEGVKKPGSSVKVSCKASGYTF |
| | scFv2 | TSYWMHWRQAPGQGLEWIGNIDPSNGETHY |
| | | NQKFDRATLTVDKSTSTAYMEGLSSLRSED |
| | | TAVYYCARERIYYSGSTYDGYFDVWGQGTT |
| | | VTVSSGSTSGSGKPGSGEGSTKDIQLTQSP |
| | | SPLSASVGDRVTITCSASSSLSYMHWYQQK |
| | | PGKSPKRWIYDTSKLASGVPSRFSGSGSGT |
| | | EYTLTISSLQPEDFATYYCQQWSSNPWTFG |
| | | GGTKVEIK |

More examples of cells that can be produced according to the present invention are provided in Table 15.

### Definitions

As used herewith “Immune cell engager” (IC engager) refers to a recombinant protein construct comprising two or more flexibly connected ligand binding domains, which are typically single chain antibodies (scFv). One of these ligand binding domains selectively binds at least one selected type of immune cells, such as T-cell, NK cell or APC. Said ligand binding domain preferably binds a “immune cells activating receptor” as defined below. The IC engager generally comprises a second binding domain that specifically binds a cell surface antigen, preferably a “antigen associated with a disease state”, which is generally chosen for being a marker of a pathological cell and for not being present at the surface of the allogeneic engineered T-cell itself. The function of the IC engager is to bring together selected types of immune cells with targeted malignant or infected cells.

Various types of soluble immune cell engagers are provided in the literature as reviewed for example by Kontermann et al. [Bispecific antibodies (2015) Drug Discovery Today 20(7):838-847], which are suitable for the methods of the present invention. As a non-limitative list, IC engagers can be bispecific T-cell engagers (BITE), dual-affinity re-targeting antibodies (DART), bispecific engagement by antibodies based on the t-cell receptor (BEAT), CROSSMAB, TRIOMAB, tandem diabody (TANDAB), ADAPTIR, affinity-tailored adaptors for t-cells (ATAC), DUOBODY, XMAB, t-cell redirecting antibody (TRAB), BICLONICS, DUTAMAB, VELOCI-BI, hinge-mutated, bispecific antibody-armed activated t-cells (AATC), bi-& tri-specific killer cell engagers (BIKE and TRIKE) as referred to in Tables 12 to 14 herein.

Tetravalent heterodimeric antibodies as described in WO2020113164 can also be used.

“Immune cell's activating receptor” refers to a receptor that triggers immune activity of immune cells such as preferably TCR for T-cells, CD16 for NK cells CD40 for APC.

“Antigen associated with a disease state” refers to an antigen present or over-expressed in a given disease. Said disease can be, for instance, a cancer or a viral infection. An antigen associated with a disease state, wherein said disease state is a cancer, i.e. “an antigen associated with a cancer” can be a tumor antigen as defined herewith.

The term “tumor antigen” is meant to cover “tumor-specific antigen” and “tumor associated antigen”. Tumor-Specific Antigens (TSA) are generally present only on tumor cells and not on any other cell, while Tumor-Associated Antigens (TAA) are present on some tumor cells and also present on some normal cells. Tumor antigen, as meant herewith, also refers to mutated forms of a protein, which only appears in that form in tumors, while the non-mutated form is observed in non-tumoral tissues. A “tumor antigen” as defined herewith also includes an antigen associated with the tumor microenvironment and/or the tumor stroma, such as for instance the Fibroblast Activation Protein (FAP) present in tumor stromal fibroblasts.

By “chimeric antigen receptor” or “CAR” is generally meant a synthetic receptor comprising a targeting moiety that is associated with one or more signalling domains in a single fusion molecule. As defined herewith, the term “chimeric antigen receptor” covers single chain CARs as well as multi-chain CARs. In general, the binding moiety of a CAR consists of an antigen-binding domain of a single-chain antibody (scFv), comprising the light and variable fragments of a monoclonal antibody joined by a flexible linker. Binding moieties based on receptor or ligand domains have also been used successfully. The signalling domains for first generation CARs are derived from the cytoplasmic region of the CD3zeta or the Fc receptor gamma chains. First generation CARs have been shown to successfully redirect T cell cytotoxicity. However, they failed to provide prolonged expansion and anti-tumor activity in vivo. Signalling domains from co-stimulatory molecules including CD28, OX-40 (CD134), and 4-1BB (CD137) have been added alone (second generation) or in combination (third generation) to enhance survival and increase proliferation of CAR modified T cells. CARs are not necessarily only single chain polypeptides, multi-chain CARs are also possible. According to the multi-chain CAR architecture, for instance as described in WO2014039523, the signalling domains and co-stimulatory domains are located on different polypeptide chains. Such multi-chain CARs can be derived from FcεRI, by replacing the high affinity IgE binding domain of FcεRI alpha chain by an extracellular ligand-binding domain such as scFv, whereas the N- and/or C-termini tails of FcεRI beta and/or gamma chains are fused to signal transducing domains and co-stimulatory domains respectively. The extracellular ligand binding domain has the role of redirecting T-cell specificity towards cell targets, while the signal transducing domains activate the immune cell response.

The term “extracellular antigen-binding domain” as used herein refers to an oligo- or poly-peptide that is capable of binding a specific antigen. Preferably, the domain will be capable of interacting with a cell surface molecule, such as a ligand. For example, the extracellular antigen-binding domain may be chosen to recognize an antigen that acts as a cell surface marker on target cells associated with a particular disease state. In a particular instance, said extracellular antigen-binding domain comprises a single chain antibody fragment (scFv) comprising the light (V_{L}) and the heavy (V_{H}) variable fragment of a target-antigen-specific monoclonal antibody joined by a flexible linker. The antigen binding domain of a CAR expressed on the cell surface of the engineered immune cells described herewith can be any domain that binds to the target antigen and that derives from, for instance, a monoclonal antibody, a recombinant antibody, a human antibody, a humanized antibody, and a functional fragment thereof.

By “immune cell” is meant a cell of hematopoietic origin functionally involved in the initiation and/or execution of innate and/or adaptative immune response, such as typically CD45, CD3 or CD4 positive cells. The immune cell described herewith may be a dendritic cell, killer dendritic cell, a mast cell, macrophage, a natural killer cell (NK-cell), cytokine-induced killer cell (CIK cell), a B-cell or a T-cell selected from the group consisting of inflammatory T-lymphocytes, cytotoxic T-lymphocytes, regulatory T-lymphocytes or helper T-lymphocytes, gamma delta T cells, Natural killer T-cell (“NKT cell).

By “allogeneic” is meant that the cells originate from a donor, or are produced and/or differentiated from stem cells in view of being infused into patients having a different haplotype. Such immune cells are generally engineered to be less alloreactive and/or become more persistent with respect to their patient host. More specifically, the method of engineering allogeneic immune cells can comprise the step of reducing or inactivating TCR expression into T-cells, or into the stem cells to be derived into T-cells. This can be obtained by different sequence specific-reagents, such as by gene silencing or gene editing techniques by using for instance nucleases, base editing techniques, shRNA and RNAi as non-limited examples.

“Originating from a donor” means that the T-cells do not necessarily come directly from the donor as fresh cells, but may derive from stem cells or cell lines obtained from initial donors, who are not the treated patient (i.e. different haplotypes).

By “primary cell” or “primary cells” are intended cells taken directly from living tissue (e.g. biopsy material) and established for growth in vitro for a limited amount of time, meaning that they can undergo a limited number of population doublings. Primary cells are opposed to continuous tumorigenic or artificially immortalized cell lines. Non-limiting examples of such cell lines are CHO-K1 cells; HEK293 cells; Caco2 cells; U2-OS cells; NIH 3T3 cells; NSO cells; SP2 cells; CHO-S cells; DG44 cells; K-562 cells, U-937 cells; MRC5 cells; IMR90 cells; Jurkat cells; HepG2 cells; HeLa cells; HT-1080 cells; HCT-116 cells; Hu-h7 cells; Huvec cells; Molt 4 cells.

Primary immune cells can be obtained from a number of non-limiting sources, including peripheral blood mononuclear cells (PBMC), bone marrow, lymph node tissue, cord blood, thymus tissue, tissue from a site of infection, ascites, pleural effusion, spleen tissue, and from tumors, such as tumor infiltrating lymphocytes. In some embodiments, said immune cell can be derived from a healthy donor, from a patient diagnosed with cancer or from a patient diagnosed with an infection. In another embodiment, said cell is part of a mixed population of immune cells which present different phenotypic characteristics, such as comprising CD4, CD8 and CD56 positive cells. Primary immune cells are provided from donors or patients through a variety of methods known in the art, as for instance by leukapheresis techniques as reviewed by Schwartz J. et al. (Guidelines on the use of therapeutic apheresis in clinical practice-evidence-based approach from the Writing Committee of the American Society for Apheresis: the sixth special issue (2013) J Clin Apher. 28(3):145-284).

The immune cells derived from stem cells are also regarded as primary immune cells according to the present invention, in particular those deriving from induced pluripotent stem cells (iPS) [Yamanaka, K. et al. (2008). “Generation of Mouse Induced Pluripotent Stem Cells Without Viral Vectors”. Science. 322 (5903): 949-53]. Lentiviral expression of reprogramming factors has been used to induce multipotent cells from human peripheral blood cells [Staerk, J. et al. (2010). “Reprogramming of human peripheral blood cells to induced pluripotent stem cells”. Cell stem cell. 7 (1): 20-4] [Loh, Y H. et al. (2010). “Reprogramming of T cells from human peripheral blood”. Cell stem cell. 7 (1): 15-9].

According to a preferred embodiment of the invention, the immune cells are derived from human embryonic stem cells by techniques well known in the art that do not involve the destruction of human embryos [Chung et al. (2008) Human Embryonic Stem Cell lines generated without embryo destruction, Cell Stem Cell 2(2):113-117].

By “Genetic engineering” is meant any methods aiming to introduce, modify and/or withdraw genetic material from a cell. By “gene editing” is meant a genetic engineering allowing genetic material to be added, removed, or altered at specific locations (loci) in the genome, including punctual mutations. Gene editing generally involves sequence specific reagents

The terms “patient” or “subject” and “donor” herein include all members of the animal kingdom including non-human primates and humans.

### Gene Editing Methods

A population of cells can be used as a starting material, such as peripheral blood mononuclear cells (PBMCs) obtained by leukapheresis, which can be submitted to a step of activation and treatment for reducing or eliminating TCR expression. This can be done with a gene editing step by using sequence specific reagents, such as for instance a rare-cutting endonuclease, to achieve stable TCR gene inactivation as described for instance with TALE-nucleases in WO2013176915.

According to one aspect of the invention, the population of genetically engineered T-cells can also be derived from [CD34]+ hematopoietic pluripotent cells, induced pluripotent stem cells (iPS), Embryonic Stem Cells (ES) or umbilical stem cells as described for instance in WO2019106163.

By “sequence-specific reagent” is meant any active molecule that has the ability to specifically recognize a selected polynucleotide sequence at a genomic locus, referred to as “target sequence”, which is generally of at least 9 bp, more preferably of at least 10 bp and even more preferably of at least 12 pb in length, in view of modifying the expression of said genomic locus. Said expression can be modified by mutation, deletion or insertion into coding or regulatory polynucleotide sequences, by epigenetic change, such as by methylation or histone modification, or by interfering at the transcriptional level by interacting with transcription factors or polymerases.

Examples of sequence-specific reagents are endonucleases, RNA guides, RNAi, methylases, exonucleases, histone deacetylases, endonucleases, end-processing enzymes such as exonucleases, and more particularly cytidine deaminases such as those coupled with the CRISPR/cas9 system to perform base editing (i.e. nucleotide substitution) without necessarily resorting to cleavage by nucleases as described for instance by Hess, G. T. et al. [Methods and applications of CRISPR-mediated base editing in eukaryotic genomes (2017) Mol Cell. 68(1): 26-43] and Liu et al. [Rees, H. A. & Liu, D. R. Base editing: precision chemistry on the genome and transcriptome of living cells. Nat. Rev. Genet. 19, 770-788 (2018)].

According to one aspect, at least 50%, preferably at least 70%, pref. at least 90%, more pref. 95% of the population express a short hairpin RNA (shRNA) or small interfering (siRNA) directed against a polynucleotide sequence encoding a component of TCR.

According to another aspect of the invention, said sequence-specific reagent is preferably a sequence-specific nuclease reagent, such as a RNA guide coupled with a guided endonuclease.

The present invention aims to improve the therapeutic potential of immune cells through gene editing techniques, especially by gene targeted integration.

By “gene targeting integration” is meant any known site-specific methods allowing to insert, replace or correct a genomic coding sequence into a living cell.

According to a preferred aspect of the present invention, said gene targeted integration involves homologous gene recombination at the locus of the targeted gene to result the insertion or replacement of at least one exogenous nucleotide, preferably a sequence of several nucleotides (i.e. polynucleotide), and more preferably a coding sequence.

By “DNA target”, “DNA target sequence”, “target DNA sequence”, “nucleic acid target sequence”, “target sequence”, or “processing site” is intended a polynucleotide sequence that can be targeted and processed by a sequence-specific nuclease reagent according to the present invention. These terms refer to a specific DNA location, preferably a genomic location in a cell, but also a portion of genetic material that can exist independently to the main body of genetic material such as plasmids, episomes, virus, transposons or in organelles such as mitochondria as non-limiting example. As non-limiting examples of RNA guided target sequences, are those genome sequences that can hybridize the guide RNA which directs the RNA guided endonuclease to a desired locus.

“Rare-cutting endonucleases” are sequence-specific endonuclease reagents of choice, insofar as their recognition sequences generally range from 10 to 50 successive base pairs, preferably from 12 to 30 bp, and more preferably from 14 to 20 bp.

According to a preferred aspect of the invention, said endonuclease reagent is a nucleic acid encoding an “engineered” or “programmable” rare-cutting endonuclease, such as a homing endonuclease as described for instance by Arnould S., et al. [WO2004067736], a zinc finger nuclease (ZFN) as described, for instance, by Urnov F., et al. [Highly efficient endogenous human gene correction using designed zinc-finger nucleases (2005) Nature 435:646-651], a TALE-Nuclease as described, for instance, by Mussolino et al. [A novel TALE nuclease scaffold enables high genome editing activity in combination with low toxicity (2011) Nucl. Acids Res. 39(21):9283-9293], or a MegaTAL nuclease as described, for instance by Boissel et al. [MegaTALs: a rare-cleaving nuclease architecture for therapeutic genome engineering (2013) Nucleic Acids Research 42(4):2591-2601].

According to another embodiment, the endonuclease reagent is a RNA-guide to be used in conjunction with a RNA guided endonuclease, such as Cas9 or Cpf1, as per, inter alia, the teaching by Doudna, J., and Chapentier, E., [The new frontier of genome engineering with CRISPR-Cas9 (2014) Science 346 (6213):1077], which is incorporated herein by reference.

According to a preferred aspect of the invention, the endonuclease reagent is transiently expressed into the cells, meaning that said reagent is not supposed to integrate into the genome or persist over a long period of time, such as be the case of RNA, more particularly mRNA, proteins or complexes mixing proteins and nucleic acids (eg: Ribonucleoproteins).

An endonuclease under mRNA form is preferably synthetized with a cap to enhance its stability according to techniques well known in the art, as described, for instance, by Kore A. L., et al. [Locked nucleic acid (LNA)-modified dinucleotide mRNA cap analogue: synthesis, enzymatic incorporation, and utilization (2009) J Am Chem Soc. 131(18):6364-5].

In general, electroporation steps that are used to transfect primary immune cells, such as PBMCs are typically performed in closed chambers comprising parallel plate electrodes producing a pulse electric field between said parallel plate electrodes greater than 100 volts/cm and less than 5,000 volts/cm, substantially uniform throughout the treatment volume such as described in WO2004083379, which is incorporated by reference, especially from page 23, line 25 to page 29, line 11. One such electroporation chamber preferably has a geometric factor (cm⁻¹) defined by the quotient of the electrode gap squared (cm2) divided by the chamber volume (cm³), wherein the geometric factor is less than or equal to 0.1 cm⁻¹, wherein the suspension of the cells and the sequence-specific reagent is in a medium which is adjusted such that the medium has conductivity in a range spanning 0.01 to 1.0 milliSiemens. In general, the suspension of cells undergoes one or more pulsed electric fields. With the method, the treatment volume of the suspension is scalable, and the time of treatment of the cells in the chamber is substantially uniform.

Due to their higher specificity, TALE-nuclease have proven to be particularly appropriate sequence specific nuclease reagents for therapeutic applications, especially under heterodimeric forms—i.e. working by pairs with a “right” monomer (also referred to as “5′” or “forward”) and ‘left” monomer (also referred to as “3″” or “reverse”) as reported for instance by Mussolino et al. [TALEN facilitate targeted genome editing in human cells with high specificity and low cytotoxicity (2014) Nucl. Acids Res. 42(10): 6762-6773].

As previously stated, the sequence specific reagent is preferably under the form of nucleic acids, such as under DNA or RNA form encoding a rare cutting endonuclease a subunit thereof, but they can also be part of conjugates involving polynucleotide(s) and polypeptide(s) such as so-called “ribonucleoproteins”. Such conjugates can be formed with reagents as Cas9 or Cpf1 (RNA-guided endonucleases) as respectively described by Zetsche, B. et al. [Cpf1 Is a Single RNA-Guided Endonuclease of a Class 2 CRISPR-Cas System (2015) Cell 163(3): 759-771], which involve RNA or DNA guides that can be complexed with their respective nucleases.

“Exogenous sequence” refers to any nucleotide or nucleic acid sequence that was not initially present at the selected locus. This sequence may be homologous to, or a copy of, a genomic sequence, or be a foreign sequence introduced into the cell. By opposition “endogenous sequence” means a cell genomic sequence initially present at a locus.

According to preferred embodiments, at least 50%, preferably at least 70%, pref. at least 90%, more pref. 95% of said engineered T-cells in the population are mutated in their TCRA, TCRB and/or CD3 alleles.

Additional genetic attributes may be conferred by gene editing to the engineered T-cells of the present invention in order to improve their therapeutic potency

In further instances, the engineered immune cell can be further modified to confer resistance to at least one immune suppressive drug, such as by inactivating CD52 that is the target of anti-CD52 antibody (e.g.: alemtuzumab), as described for instance in WO2013176915.

In further instances, the engineered immune cell can be further modified to confer resistance to and/or a chemotherapy drug, in particular a purine analogue drug, for example by inactivating DCK as described in WO201575195.

In further instances, the engineered immune cell can be further modified to improve its persistence or its lifespan into the patient, in particular inactivating a gene encoding MHC-I component(s) such as HLA or β2m, such as described in WO2015136001 or by Liu et al. (2017, Cell Res 27:154-157).

In still further instances, the engineered immune cell is mutated to improve its CAR-dependent immune activation, in particular to reduce or suppress the expression of immune checkpoint proteins and/or their receptors thereof, such as PD1 or CTLA4 as described in WO2014184744.

In further instances, such as in example 3 herein, the invention comprises integrating into immune cells a transgene encoding an immune cell engager at a locus encoding Granulocyte-Macrophage Colony-Stimulating Factor (GM-CSF) protein, preferably in view of inactivating expression of GM-CSF. This inactivation has, among others, the effect of lowering the risk of cytokine release syndrome (CRS) and neuroinflammation induced by cytokines, such as IL-6, MCP-1, and IL-8. These cytokines are generally produced by myeloid cells upon detection of GM-CSF secreted by the activated T-cells. For instance, the invention can be drawn to engineered cells that have integrated a transgene encoding a CAR and an immune cell engager at a GM-CSF locus endogenous locus.

In still further instances, the CAR-T cells of the invention can be genetically engineered in order to reduce or inactivate expression of the surface antigen targeted by the CAR to avoid fratricide killing. As an example, a CAR-T targeting CS1 antigen tumor (CAR CS1) can have its endogenous CS1 gene inactivated by using a rare-cutting endonuclease.

Non-limitative examples of TALE-nuclease targeting endogenous genes expressing TRAC, CD52, B2M, GM-CSF and CS1 are provided in Table 1 and 16. The invention can be practiced as described herein with such polynucleotides or polypeptides having at least 70%, preferably 80%, more preferably 90% and even more preferably 95 or 99% identity with the sequences referred to in Table 2.

TABLE 2
example of preferred endonuclease target
sequences and TALE-nucleases

| Targeted | SEQ | |
|---|---|---|
| Genomic | ID | |
| regions | # | Target sequence |
| trac | 46 | See sequence listing |
| target | | |
| region | | |
| cd52 | 47 | See sequence listing |
| target | | |
| region | | |
| b2m | 48 | See Sequence listing |
| target | | |
| region | | |
| cs1 | 49 | See sequence listing |
| target | | |
| region | | |
| TRAC_T01- | 50 | TTGTCCCACAGATATCCaga |
| target | | accctgaccctgCCGTGTAC |
| | | CAGCTGAGA |
| CD52_T01- | 51 | TTCCTCCTACTCACCATcag |
| target | | cctcctggttatGGTACAGG |
| | | TAAGAGCAA |
| B2M_T01- | 52 | TTAGCTGTGCTCGCGCTact |
| target | | ctctctttctGGCCTGGAGG |
| | | CTATCCA |
| CS1_T01- | 53 | TATATCCTTTGGCAGCTcac |
| target | | aggtgagtccGGCCGGATTC |
| | | TCTTCCA |
| TRAC_T01-L | 23 | TALEN polypeptide |
| | | See sequence listing |
| TRAC_T01-R | 24 | TALEN polypeptide |
| | | See sequence listing |
| | 25 | TALEN polypeptide |
| CD52_T01-L | | See sequence listing |
| CD52_T01-R | 26 | TALEN polypeptide |
| | | See |
| | | Sequence listing |
| B2M_T01-L4 | 27 | TALEN polypeptide |
| | | See |
| | | Sequence listing |
| B2M_T01-R4 | 28 | TALEN polypeptide |
| | | See |
| | | Sequence listing |
| CS1_T01-L2 | 29 | TALEN polypeptide |
| | | See |
| | | Sequence listing |
| CS1_T01-R2 | 30 | TALEN polypeptide |
| | | See |
| | | Sequence listing |

In further embodiments, the engineered immune cell can be further modified to obtain co-expression in said cell of another exogenous genetic sequence selected from one encoding:
NK cell inhibitor, such as HLAG, HLAE or ULBP1;
CRS inhibitor, such as is a mutated IL6Ra, sGP130 or IL18-BP;
Cytochrome(s) P450, CYP2D6-1, CYP2D6-2, CYP2C9, CYP3A4, CYP2C19 or CYP1A2, conferring hypersensitivity of said immune cells to a drug, such as cyclophosphamide and/or isophosphamide;
Dihydrofolate reductase (DHFR), inosine monophosphate dehydrogenase 2 (IMPDH2), calcineurin or methylguanine transferase (MGMT), mTORmut or Lckmut, conferring drug resistance;
Chemokine or a cytokine, such as IL-2, IL-12 and IL-15;
Chemokine receptors, such as CCR2, CXCR2, or CXCR4; and/or
a secreted inhibitor of Tumor Associated Macrophages (TAM), such as a CCR2/CCL2 neutralization agent, to enhance the therapeutic activity of the immune cells;

According to the present invention the exogenous polynucleotide sequences for expression of the immune cell engager, as well as the other above exogenous optional sequences are preferably integrated at a locus regulated by or encoding TCR, HLA, β2m, PD1, CTLA4, TIM3, LAG3, CD69, GM-CSF, IL2Ra and/or CD52.

As one object of the present invention, the AAV vector used in the method can comprise a 2A peptide cleavage site followed by the cDNA (minus the start codon) forming the exogenous coding sequence.

As a preferred object, said AAV vector can further comprises an exogenous sequence coding for a chimeric receptor, for instance a chimeric antigen receptor (CAR), especially an anti-CD19 CAR, an anti-CD22 CAR, an anti-CD123 CAR, an anti-CS1 CAR, an anti-CCL1 CAR, an anti-MUC1 CAR, an anti-MSLN CAR or an anti-CD20 CAR, which can be co-expressed with the IC engagers.

Gene targeted insertion of the sequences encoding IC engagers as well as CARs and other exogenous genetic sequences can be performed by using AAV vectors, especially vectors from the AAV6 family or chimeric vectors AAV2/6 previously described by Sharma A., et al. [Transduction efficiency of AAV 2/6, 2/8 and 2/9 vectors for delivering genes in human corneal fibroblasts. (2010) Brain Research Bulletin. 81 (2-3): 273-278].

One aspect of the present invention is thus the transduction of such AAV vectors encoding IC engagers in human primary T-cells, in conjunction with the expression of sequence-specific endonuclease reagents, such as TALE endonucleases, to increase gene integration at the loci previously cited.

According to a preferred aspect of this invention, sequence specific endonuclease reagents can be introduced into the cells by transfection, more preferably by electroporation of mRNA encoding said sequence specific endonuclease reagents.

Accordingly, the invention provides with a method for inserting an exogenous nucleic acid sequence coding for an IC engager at one of the previous selected locus, which comprising at least one of the following steps:
transducing into said cell an AAV vector comprising said exogenous nucleic acid sequence encoding IC engager and the sequences homologous to the targeted endogenous DNA sequence, and optionally
Inducing the expression of a sequence specific endonuclease reagent to cleave said endogenous sequence at the locus of insertion.

The obtained insertion of the exogenous nucleic acid sequence may result into the introduction of genetic material, correction or replacement of the endogenous sequence, more preferably “in frame” with respect to the endogenous gene sequences at that locus, but also to the inactivation of the endogenous locus.

As one object of the present invention, the AAV vector used in the method can comprise an exogenous coding sequence that is “promoterless”, said coding sequence being any of those referred to in this specification.

Many other vectors known in the art, such as plasmids, episomal vectors, linear DNA matrices, etc. . . . can also be used to perform gene insertions at those loci by following the teachings of the present invention.

As stated before, the DNA vector used for gene integration according to the invention preferably comprises: (1) said exogenous nucleic acid to be inserted comprising the exogenous coding sequence of IC engager, and (2) a sequence encoding the sequence specific endonuclease reagent that promotes said insertion. According to a more preferred aspect, said exogenous nucleic acid under (1) does not comprise any promoter sequence, whereas the sequence under (2) has its own promoter. According to an even more preferred aspect, the nucleic acid under (1) comprises an Internal Ribosome Entry Site (IRES) or “self-cleaving” 2A peptides, such as T2A, P2A, E2A or F2A, so that the endogenous gene where the exogenous coding sequence is inserted becomes multi-cistronic. The IRES of 2A Peptide can precede or follow said exogenous coding sequence.

The integration of the exogenous polynucleotide sequences for expression of said immune cell engager of the present invention can also be introduced into the T cells by using a viral vector, in particular lentiviral vectors. The present invention thus provides with viral vectors encoding immune cell engagers as described herein.

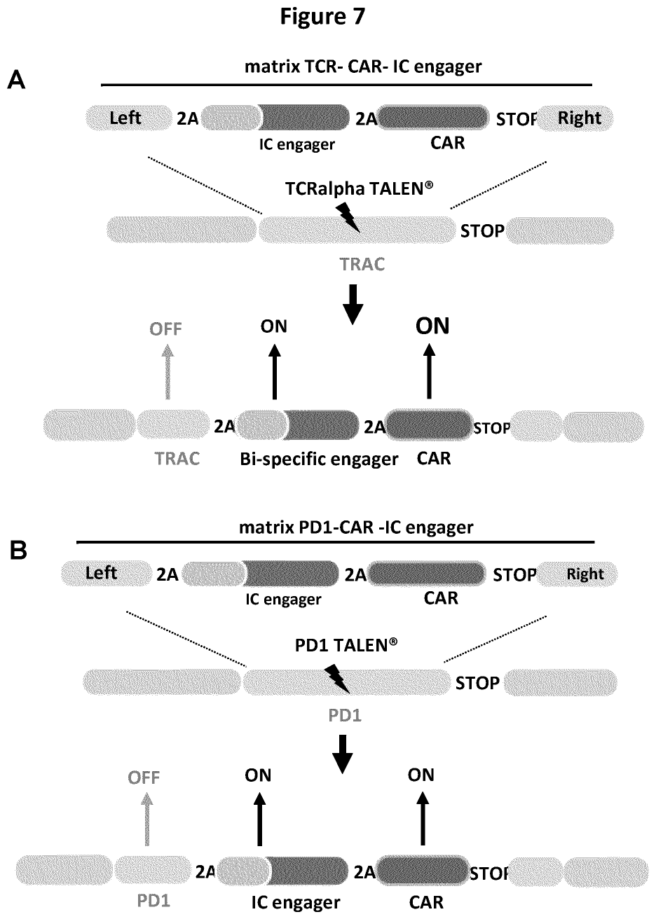

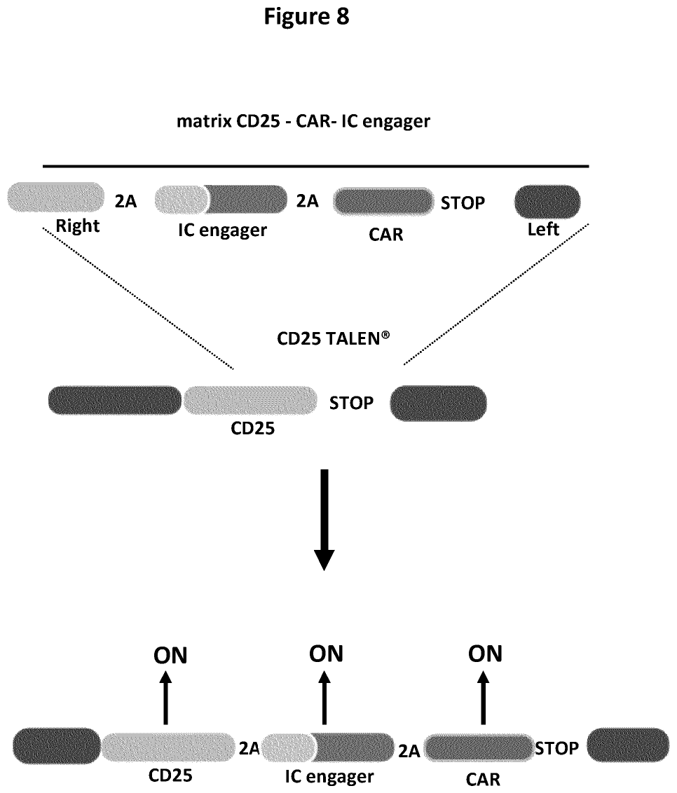

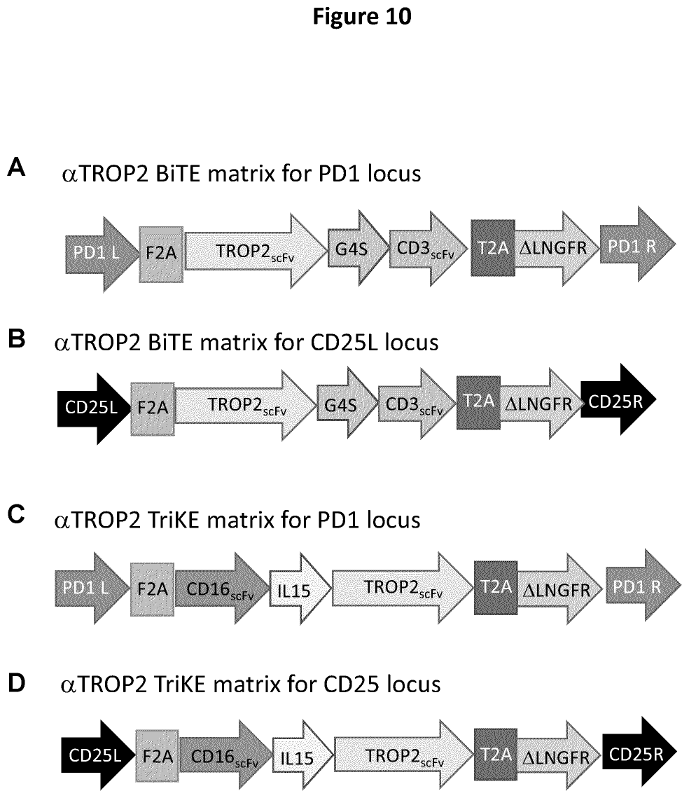

Lentiviral or AAV vectors according to the invention preferably comprise both sequences encoding IC engager (s) and CAR(s) separated by a T2A or P2A sequence as illustrated in FIGS. 7 to 10, as forming one transcriptional unit. In lentiviral vectors said sequences generally form an expression cassette transcribed under control of a constitutive exogenous promoter, such as a EF1alpha promoter derived from the human EEF1A1 gene.

### Antigen-Specific CARs

In one aspect of the invention, the allogeneic immune cells are endowed with a synthetic CAR which confers them a higher specificity toward specific cell antigen(s), including specificity toward malignant cells, or the tumor microenvironment, toward infected cells or inflammatory tissues. A recombinant receptor is generally encoded by an exogenous polynucleotide which is introduced into the cell using vectors as per one of the transduction steps referred to elsewhere in the current application. A recombinant receptor encoded by an exogenous polynucleotide can also be introduced into the cell in the form of a plasmid or a PCR product.

In one aspect, the CAR expressed by these cells specifically targets an antigen marker at the surface of malignant or infected cells, which further helps said immune cells to destroy these cells in-vivo as reviewed by Sadelain M. et al (2013) Cancer Discov. 3(4):388-98.

In another aspect, the CAR expressed by these cells specifically targets an antigen marker at the surface of cells comprised in the tumor stroma, such as the Fibroblast Activation Protein (FAP) present in tumor stromal fibroblasts.

In general, CAR polypeptides comprise an extracellular antigen-binding domain, a transmembrane domain, and an intracellular domain comprising a costimulatory domain and/or a primary signalling domain, wherein said antigen binding domain binds to the antigen associated with the disease state.

While the method described herewith is not limited to a specific CAR structure, nor on a specific CAR, a nucleic acid that can be used to engineer the immune cells generally encodes a CAR comprising: an extracellular antigen-binding domain that binds to an antigen associated with a disease state, a hinge, a transmembrane domain, and an intracellular domain comprising a stimulatory domain and/or a primary signalling domain. Generally, the extracellular antigen-binding domain is a scFv comprising a Heavy variable chain (VH) and a Light variable chain (VL) of an antibody binding to a specific antigen (e.g., to a tumor antigen) connected via a Linker. The transmembrane domain can be, for example, a CD8α transmembrane domain or a 4-1BB transmembrane domain. The stimulatory domain can be, for example, the 4-1BB stimulatory domain. The primary signalling domain can be, for example, the CD3ζ signalling domain.

TABLE 3
Sequence of different domains typically present in a CAR

| Functional domains | SEQ ID # | amino acid sequence |
|---|---|---|
| CD8a signal | SEQ ID NO.1 | MALPVTALLLPLALLLHAARP |
| peptide (or | | |
| sequence leader) | | |
| Alternative signal | SEQ ID NO.2 | METDTLLLWVLLLWWVPGSTG |
| peptide | | |
| FcγRIllα hinge | SEQ ID NO.3 | GLAVSTISSFFPPGYQ |
| CD8a hinge | SEQ ID NO.4 | TTTPAPRPPTPAPTIASQPLSLRPEACRPAAGGAVH |
| | | TRGLDFACD |
| IgG1 hinge | SEQ ID NO.5 | EPKSPDKTHTCPPCPAPPVAGPSVFLFPPKPKDTL |
| | | MIARTPEVTCVVVDVSHEDPEVKFNWYVDGVEVHN |
| | | AKTKPREEQYNSTYRVVSVLTVLHQDWLNGKEYKC |
| | | KVSNKALPAPIEKTISKAKGQPREPQVYTLPPSRDE |
| | | LTKNQVSLTCLVKGFYPSDIAVEWESNGQPENNYK |
| | | TTPPVLDSDGSFFLYSKLTVDKSRWQQGNVFSCSV |
| | | MHEALHNHYTQKSLSLSPGK |
| CD8a | SEQ ID NO. 6 | IYIWAPLAGTCGVLLLSLVITLYC |
| transmembrane | | |
| domain | | |
| 4-1BB | SEQ ID NO. 7 | IISFFLALTSTALLFLLFFLTLRFSVV |
| transmembrane | | |
| domain | | |
| 4-1BB stimulatory | SEQ ID NO. 8 | KRGRKKLLYIFKQPFMRPVQTTQEEDGCSCRFPEE |
| domain | | EEGGCEL |
| CD37 signalling | SEQ ID NO. 9 | RVKFSRSADAPAYQQGQNQLYNELNLGRREEYDV |
| domain | | LDKRRGRDPEMGGKPRRKNPQEGLYNELQKDKM |
| | | AEAYSEIGMKGERRRGKGHDGLYQGLSTATKDTY |
| | | DALHMQALPPR |
| Linker | SEQ ID NO. 10 | GGGGSGGGGSGGGGS |

An example of a CAR targeting the CD123 antigen present on tumor cells used to illustrate the present invention is described in Tables 4 and 5 below and in the Example section.

TABLE 4
Sequence of the CD123 VH and VL comprised
in the ScFv of preferred CD123 CAR

| ScFv | SEQ ID | |
|---|---|---|
| sequences | # | amino acid sequence |
| Heavy | SEQ ID | EVKLVESGGGLVQPGGSLSLS |
| variable | NO. 11 | CAASGFTFTDYYMSWVRQPPG |
| region | | KALEWLALIRSKADGYTTEYS |
| | | ASVKGRFTLSRDDSQSILYLQ |
| | | MN |
| | | ALRPEDSATYYCARDAAYYSY |
| | | YSPEGAMDYWGQGTSVTVSS |
| Light | SEQ ID | MADYKDIVMTQSHKFMSTSVG |
| variable | NO. 12 | DRVNITCKASQNVDSAVAWYQ |
| region | | QKPGQSPKALIYSASYRYSGV |
| | | PDRFTGRGSGTDFTLTISSVQ |
| | | AE |
| | | DLAVYYCQQYYSTPWTFGGGT |
| | | KLEIKR |

TABLE 5
Structure of preferred CD123 CAR

| CAR | CAR Structure | | | | | | |
|---|---|---|---|---|---|---|---|
| Designation | signal | | | CD8α | CD8α | 4-1BB | CD3ζ |
| 123 CAR | peptide | VH | VL | hinge | TM | IC | CD |
| (SEQ ID | SEQ ID | SEQ ID | SEQ ID | SEQ ID | SEQ ID | SEQ ID | SEQ ID |
| NO. 13) | NO. 1 | NO. 11 | NO. 12 | NO. 4 | NO. 6 | NO. 8 | NO. 9 |

An example of a CAR targeting the CS1 antigen present on tumor cells used to illustrate the present invention is described in Tables 4 and 5 below and in the Example section.

TABLE 6
Sequence of the CS1 VH and
VL comprised in the ScFv
of preferred CS1 CAR

| ScFv | SEQ ID | amino acid |
|---|---|---|
| sequences | # | sequence |
| Heavy | SEQ ID | QVQLQQPGAELVRPGASVKLSCKAS |
| variable | NO. 14 | GYSFTTYWMNWVKQRPGQGLEWIGM |
| region | | IHPSDSETRLNQKFKDKATLTVDKS |
| | | SSTAYMQLSSPTSEDSAVYYCARST |
| | | MIATRAMDYWGQGTSVTVSS |
| Light | SEQ ID | DIVMTQSQKSMSTSVGDRVSITCKA |
| variable | NO. 15 | SQDVITGVAWYQQKPGQSPKLLIYS |
| region | | ASYRYTGVPDRFTGSGSGTDFTFTI |
| | | SNVQAEDLAVYYCQQHYSTPLTFGA |
| | | GTKLELK |

TABLE 7
Structure of preferred CS1 CAR

| CAR | CAR Structure | | | | | | |
|---|---|---|---|---|---|---|---|
| Designation | signal | | | CD8α | CD8α | 4-1BB | CD3ζ |
| CS1 CAR | peptide | VH | VL | hinge | TM | IC | CD |
| (SEQ ID | SEQ ID | SEQ ID | SEQ ID | SEQ ID | SEQ ID | SEQ ID | SEQ ID |
| NO. 16) | NO. 1 | NO. 14 | NO. 15 | NO. 4 | NO. 6 | NO. 8 | NO. 9 |

An example of a CAR targeting the CLL1 antigen present on tumor cells used to illustrate the present invention is described in Tables 8 and 9 below and in the Example section.

TABLE 8
Sequence of the CLL1 VH and VL comprised
in the ScFv of preferred CLL1 CAR

| ScFv | SEQ ID | |
|---|---|---|
| sequences | # | amino acid sequence |
| Heavy | SEQ ID | EVQLQQSGPELVKPGASVKMSCKAS |
| variable | NO. 17 | GYTFTSYFIHWVKQKPGQGLEWIGF |
| region | | INPYNDGSKYNEKFKGKATLTSDKS |
| | | SSTAYMELSSLTSEDSAVYYCTRDD |
| | | GYYGYAMDYWGQGTSVTVSS |
| Light | SEQ ID | DIQMTQSPSSLSASLGERVSLTCRA |
| variable | NO. 18 | TQELSGYLSWLQQKPDGTIKRLIYA |
| region | | ASTLDSGVPKRFSGNRSGSDYSLTI |
| | | SSLESEDFADYYCLQYAIYPYTFGG |
| | | GTKLEIK |

TABLE 9
Structure of preferred CLL1 CAR

| CAR | CAR Structure | | | | | | |
|---|---|---|---|---|---|---|---|
| Designation | signal | | | CD8α | CD8α | 4-1BB | CD3ζ |
| CLL1 CAR | peptide | VH | VL | hinge | TM | IC | CD |
| (SEQ ID | SEQ ID | SEQ ID | SEQ ID | SEQ ID | SEQ ID | SEQ ID | SEQ ID |
| NO. 19) | NO. 1 | NO. 17 | NO. 18 | NO. 4 | NO. 6 | NO. 8 | NO. 9 |

An example of a CAR targeting the CD22 antigen present on tumor cells used to illustrate the present invention is described in Tables 10 and 11 below and in the Example section.

TABLE 10
Sequence of the CD22 VH and VL comprised
in the ScFv of preferred CD22 CAR T

| ScFv | SEQ ID | |
|---|---|---|
| sequences | # | amino acid sequence |
| Heavy | SEQ ID | QVQLQQSGPGLVKPSQTLSLTCAIS |
| variable | NO. 20 | GDSVSSNSAAWNWIRQSPSRGLEWL |
| region | | GRTYYRSKWYNDYAVSVKSRITINP |
| | | DTSKNQFSLQLNSVTPEDTAVYYCA |
| | | REVTGDLEDAFDIWGQGTMVTVSS |
| Light | SEQ ID | DIQMTQSPSSLSASVGDRVTITCRA |
| variable | NO. 21 | SQTIWSYLNWYQQRPGKAPNLLIYA |
| region | | ASSLQSGVPSRFSGRGSGTDFTLTI |
| | | SSLQAEDFATYYCQQSYSIPQTFGQ |
| | | GTKLEIK |

TABLE 11
Structure of preferred CD22 CAR

| CAR | CAR Structure | | | | | | |
|---|---|---|---|---|---|---|---|
| Designation | signal | | | CD8α | CD8α | 4-1BB | CD3ζ |
| CD22 CAR | peptide | VH | VL | hinge | TM | IC | CD |
| (SEQ ID | SEQ ID | SEQ ID | SEQ ID | SEQ ID | SEQ ID | SEQ ID | SEQ ID |
| NO. 22) | NO. 1 | NO. 20 | NO. 21 | NO. 4 | NO. 6 | NO. 8 | NO. 9 |

The CAR expressed on the surface of an engineered immune cell described herewith generally binds to specific epitope(s) of an antigen associated to, or mainly expressed in, a pathological cell like a tumor cell, or to an antigen associated with the tumor stroma, or to an antigen associated to a virus. As a result, the CAR-expressing immune cells specifically recognize and bind antigens present on the surface of the target cell and kill the cell. In particular the CAR-expressing immune cells targeting tumor cells can kill the tumor cells.

Many CARs have been described in the art, which can be used to carry out the present method, or to prepare the engineered cells useful in the invention. In particular, such CARs can bind tumor antigens as diverse as one selected from: Interleukin 3 receptor subunit alpha.spanning 4-domains A1 (MS4A1 also known as CD20); CD22 molecule (CD22); CD229 molecule (CD229) CD24 molecule (CD24); CD248 molecule (CD248); CD276 molecule (CD276 or B7H3); CD3 molecule (CD3); CD33 molecule (CD33); CD38 molecule (CD38); CD44v6; CD5 molecule (CD5); CD56 molecule (CD56); CD7 molecule (CD7); CD70 molecule (CD70); CD72; CD79a; CD79b; TNF receptor superfamily member 8 (TNFRSF8 also known as CD30); KIT proto-oncogene receptor tyrosine kinase (CD117); V-set pre-B cell surrogate light chain 1 (VPREB1 or CD179a); adhesion G protein-coupled receptor E5 (ADGRE5 or CD97); TNF receptor superfamily member 17 (TNFRSF17 also known as BCMA); SLAM family member 7 (SLAMF7 also known as CS1); L1 cell adhesion molecule (L1CAM); C-type lectin domain family 12 member A (CLEC12A also known as CLL-1); tumor-specific variant of the epidermal growth factor receptor (EGFRvIII); thyroid stimulating hormone receptor (TSHR); Fms related tyrosine kinase 3 (FLT3); ganglioside GD3 (GD3); Tn antigen (Tn Ag); lymphocyte antigen 6 family member G6D (LY6G6D); Delta like canonical Notch ligand 3 (DLL3); Interleukin-13 receptor subunit alpha-2 (IL-13RA2); Interleukin 11 receptor subunit alpha (IL11RA); mesothelin (MSLN); Receptor tyrosine kinase like orphan receptor 1 (ROR1); Prostate stem cell antigen (PSCA); erb-b2 receptor tyrosine kinase 2 (ERBB2 or Her2/neu); Protease Serine 21 (PRSS21); Kinase insert domain receptor (KDR also known as VEGFR2); Lewis y antigen (LewisY); Solute carrier family 39 member 6 (SLC39A6); Fibroblast activation protein alpha (FAP); Hsp70 family chaperone (HSP70); Platelet-derived growth factor receptor beta (PDGFR-beta); Cholinergic receptor nicotinic alpha 2 subunit (CHRNA2); Stage-Specific Embryonic Antigen-4 (SSEA-4); Mucin 1, cell surface associated (MUC1); mucin 16, cell surface associated (MUC16); claudin 18 (CLDN18); claudin 6 (CLDN6); Epidermal Growth Factor Receptor (EGFR); Preferentially expressed antigen in melanoma (PRAME); Neural Cell Adhesion Molecule (NCAM); ADAM metallopeptidase domain 10 (ADAM10); Folate receptor 1 (FOLR1); Folate receptor beta (FOLR2); Carbonic Anhydrase IX (CA9); Proteasome subunit beta 9 (PSMB9 or LMP2); Ephrin receptor A2 (EphA2); Tetraspanin 10 (TSPAN10); Fucosyl GM1 (Fuc-GM1); sialyl Lewis adhesion molecule (sLe); TGS5; high molecular weight-melanoma-associated antigen (HMWMAA); o-acetyl-GD2 ganglioside (OAcGD2); tumor endothelial marker 7-related (TEM7R); G protein-coupled receptor class C group 5, member D (GPRC5D); chromosome X open reading frame 61 (CXORF61); ALK receptor tyrosine kinase (ALK); Polysialic acid; Placenta-specific 1 (PLAC1); hexasaccharide portion of globoH glycoceramide (GloboH); NY-BR-1 antigen; uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 family member K (LY6K); olfactory receptor family 51 subfamily E member 2 (OR51E2); TCR Gamma Alternate Reading Frame Protein (TARP); Wilms tumor protein (WT1); ETV6-AML1 fusion protein due to 12; 21 chromosomal translocation (ETV6-AML1); sperm autoantigenic protein 17 (SPA17); X Antigen Family, Member 1E (XAGE1E); TEK receptor tyrosine kinase (Tie2); melanoma cancer testis antigen-1 (MAD-CT-1); melanoma cancer testis antigen-2 (MAD-CT-2); Fos-related antigen 1; p53 mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B 1; v-myc avian myelocytomatosis viral oncogene neuroblastoma derived homolog (MYCN); Ras Homolog Family Member C (RhoC); Cytochrome P450 1B 1 (CYP1B 1); CCCTC-Binding Factor (Zinc Finger Protein)-Like (BORIS); Squamous Cell Carcinoma Antigen Recognized By T Cells 3 (SART3); Paired box protein Pax-5 (PAX5); proacrosin binding protein sp32 (OY-TES 1); lymphocyte-specific protein tyrosine kinase (LCK); A kinase anchor protein 4 (AKAP-4); synovial sarcoma, X breakpoint 2 (SSX2); Leukocyte-associated immunoglobulin-like receptor 1 (LAIR1); Fc fragment of IgA receptor (FCAR); Leukocyte immunoglobulin-like receptor subfamily A member 2 (LILRA2); CD300 molecule-like family member f (CD300LF); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1).

CARs of particular interest in the method described herewith comprise an extracellular binding domain directed against an antigen selected from CD123, CD19, CD20, CD22, CD33, 5T4, ROR1, CD38, CS1, BCMA, Flt3, CD70, EGFRvIII, WT1, HSP-70, CLL1, MUC1, ERBB2, and MSLN. Such CARs can have the structure described in WO2016120216.

More particularly, CARs expressed by the immune cells on which the methods and kits described herewith can apply comprise an extracellular binding domain directed against an antigen selected from CD123, CD22, CS1, CLL1, MUC1, and mesothelin (MSLN).

The method and kits described herewith can be applied to any immune cell genetically engineered to express a synthetic chimeric antigen receptor, in particular a chimeric antigen receptor targeting an antigen associated with a disease state such as a tumor antigen or a viral antigen.

In a more particular instance, the genetically engineered immune cell expresses one or more CARs targeting an antigen associated with a cancer such as a tumor-specific antigen, a tumor-associated antigen and/or an antigen associated with the tumor microenvironment and/or the tumor stroma.

In another instance, the genetically engineered immune cell expresses one of more CARs targeting an antigen selected from the group consisting of CD123, CD19, CD20, CD22, CD33, 5T4, ROR1, CD38, CS1, BCMA, Flt3, CD70, EGFRvIII, WT1, HSP-70, CLL1, MUC1, ERBB2, and MSLN.

Stable expression of CARs in said immune cells can be achieved using, for example, viral vectors (e.g., lentiviral vectors, retroviral vectors, Adeno-Associated Virus (AAV) vectors) or transposon/transposase systems or plasmids or PCR products integration. Other approaches include direct mRNA electroporation.

The applicant has formerly made available robust protocols and gene editing strategies to produce allogeneic therapeutic grade T-cells from PBMCs, especially by providing very safe and specific endonuclease reagents under the form of TALE-nucleases (TALEN®). The production of so-called “universal T-cells”, which are [TCR]^{neg} T-cells from donors was achieved and successfully injected to patients with reduced Graft versus Host Disease (GVhD) (Poirot et al. 2015, Cancer. Res. 75 (18): 3853-3864; Qasim et al., 2017, Science Translational 9(374)). Meanwhile, inactivation of TCR or β2m components in primary T-cells can be combined with the inactivation of further genes encoding checkpoint inhibitor proteins, such as described for instance in WO2014184744.

In further instances, the engineered immune cell can be further modified to confer resistance to at least one immune suppressive drug, such as by inactivating CD52 that is the target of anti-CD52 antibody (e.g.: alemtuzumab), as described for instance in WO 2013176915.

In further instances, the engineered immune cell can be further modified to confer resistance to and/or a chemotherapy drug, in particular a purine analogue drug, for example by inactivating DCK as described in WO201575195.

In further instances, the engineered immune cell can be further modified to improve its persistence or its lifespan into the patient, in particular inactivating a gene encoding MHC-I component(s) such as HLA or β2m, such as described in WO2015136001 or by Liu et al. (2017, Cell Res 27:154-157).

In still further instances, the engineered immune cell is mutated to improve its CAR-dependent immune activation, in particular to reduce or suppress the expression of immune checkpoint proteins and/or their receptors thereof, such as PD1 or CTLA4 as described in WO 2014184744.

### Activation and Expansion of T Cells

Whether prior to or after genetic modification, the immune cells according to the present invention can be activated or expanded, even if they can activate or proliferate independently of antigen binding mechanisms. T-cells, in particular, can be activated and expanded using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 2006/0121005. T cells can be expanded in vitro or in vivo. T cells are generally expanded by contact with an agent that stimulates a CD3 TCR complex and a co-stimulatory molecule on the surface of the T cells to create an activation signal for the T-cell. For example, chemicals such as calcium ionophore A23187, phorbol 12-myristate 13-acetate (PMA), or mitogenic lectins like phytohemagglutinin (PHA) can be used to create an activation signal for the T-cell.

As non-limiting examples, T cell populations may be stimulated in vitro such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. Conditions appropriate for T cell culture include an appropriate media (e.g., Minimal Essential Media or RPMI Media 1640 or, X-vivo 5, (Lonza)) that may contain factors necessary for proliferation and viability, including serum (e.g., fetal bovine or human serum), interleukin-2 (IL-2), insulin, IFN-g, IL-4, IL-7, GM-CSF, IL-10, IL-12, IL-15, TGFp, and TNF- or any other additives for the growth of cells known to the skilled artisan. Other additives for the growth of cells include, but are not limited to, surfactant, plasmanate, and reducing agents such as N-acetyl-cysteine and 2-mercaptoethanoi. Media can include RPMI 1640, A1M-V, DMEM, MEM, a-MEM, F-12, X-Vivo 1, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO2). T-cells that have been exposed to varied stimulation times may exhibit different characteristics

In another particular embodiment, said cells can be expanded by co-culturing with tissue or cells. Said cells can also be expanded in vivo, for example in the subject's blood after administrating said cell into the subject.

Any biological activity exhibited by the engineered immune cell expressing a CAR can be determined, including, for instance, cytokine production and secretion, degranulation, proliferation, or any combination thereof.

In a particular instance, the biological activity determined in step (iii) is cytokine secretion, cell proliferation, or both.

Said biological activities can be measured by standard methods well known by the skilled person, in particular by in vitro and/or ex vivo methods.

Secretion of any cytokine can be measured, in particular secretion of IFNγ, TNFα, can be determined. Standard methods to determine cytokine secretion includes ELISA, flow cytometry. These methods are described for instance in Sachdeva et al. (Front Biosci, 2007, 12: 4682-95) and Pike et al (2016) (Methods in Molecular Biology, vol 1458. Humana Press, New York, N.Y.).

The level of cytokine secretion can be measured, for instance, as the maximum level of cytokine (e.g., IFNγ) secreted per CAR-expressing immune cell (e.g., CAR-T cell), e.g. maximum amount of IFNγ secreted per CAR-T cell.

To evaluate “degranulation”, standard methods can be used, including for instance CD107a degranulation assay or measurement of secreted Granzyme B or Perforin (such as described in Lorenzo-Herrero et al, [Methods Mol Biol (2019) 1884:119-130; Betts et al. Methods in Cell Biology (2004) 75:497-512].

To evaluate “proliferation” activity, standard methods can be carried out, which are mainly based on methods involving measurement of DNA synthesis, detection of proliferation-specific markers, measurement of successive cell divisions by the use of cell membrane binding dyes, measurement of cellular DNA content and measurement of cellular metabolism.

### Therapeutic Compositions and Applications

The method of the present invention described above allows producing engineered primary immune cells within a limited time frame of about 15 to 30 days, preferably between and 20 days, and most preferably between 18 and 20 days so that they keep their full immune therapeutic potential, especially with respect to their cytotoxic activity.

These cells can form or be members of populations of cells, which preferably originate from a single donor or patient. These populations of cells can be expanded under closed culture recipients to comply with highest manufacturing practices requirements and can be frozen prior to infusion into a patient, thereby providing “off the shelf” or “ready to use” therapeutic compositions.

As per the present invention, a significant number of cells originating from the same Leukapheresis can be obtained, which is critical to obtain sufficient doses for treating a patient. Although variations between populations of cells originating from various donors may be observed, the number of immune cells procured by a leukapheresis is generally about from 10⁸ to 10¹⁰ cells of PBMC. PBMC comprises several types of cells: granulocytes, monocytes and lymphocytes, among which from 30 to 60% of T-cells, which generally represents between 10⁸ to 10⁹ of primary T-cells from one donor. The method of the present invention generally ends up with a population of engineered cells that reaches generally more than about 10⁸ T-cells, more generally more than about 10⁹ T-cells, even more generally more than about 10¹⁰ T-cells, and usually more than 10¹¹ T-cells. In general, the T-cells are gene edited at least at two different loci.

Such cells, compositions or populations of cells can therefore be used as a medicament; especially for treating cancer, particularly for the treatment of lymphoma, but also for solid tumors such as melanomas, neuroblastomas, gliomas or carcinomas such as lung, breast, colon, prostate or ovary tumors in a patient in need thereof.

The invention is more particularly drawn to populations of primary TCR negative T-cells originating from a single donor, wherein at least 20%, preferably 30%, more preferably 50% of the cells in said population have been modified using sequence-specific reagents in at least two, preferably three different loci.

Alternatively, the engineered cells of the present invention can be gamma-delta T-cells used in allogeneic settings.

More specifically, the present invention discloses populations of immune cells as described herein, wherein at least 20%, preferably at least 30%, 40%, 50%, 60%, or even 70%, and more preferably at least 80% of the cells have integrated a transgene encoding an immune cell engager, and optionally a chimeric antigen receptor or a recombinant TCR.

In another aspect, the present invention relies on methods for treating patients in need thereof, said method comprising at least one of the following steps:
(a) Determining specific antigen markers present at the surface of patients tumors biopsies;
(b) providing a population of engineered primary immune cells originating from a donor engineered by one of the methods of the present invention previously described expressing a CAR directed against said specific antigen markers;
(c) selecting and expressing in said population of engineered immune cells an IC engager as previously described, directed against at least one of said antigen markers, which can be the same as the CAR or a different one;
(d) Administrating said engineered population of engineered primary immune cells to said patient,

Generally, said populations of cells mainly comprises CD4 and CD8 positive immune cells, such as T-cells, which can undergo robust in vivo T cell expansion and can persist for an extended amount of time in-vitro and in-vivo.

The treatments involving the engineered primary immune cells according to the present invention can be ameliorating, curative or prophylactic.

In another embodiment, said isolated cell according to the invention or cell line derived from said isolated cell can be used for the treatment of liquid tumors, and preferably leukemia.

Adult tumors/cancers and pediatric tumors/cancers are also included.

The treatment with the engineered immune cells according to the invention may be in combination with one or more therapies against cancer selected from the group of antibodies therapy, chemotherapy, cytokines therapy, dendritic cell therapy, gene therapy, hormone therapy, laser light therapy and radiation therapy.

According to a preferred embodiment of the invention, said treatment can be administrated into patients undergoing an immunosuppressive treatment. Indeed, the present invention preferably relies on cells or population of cells, which have been made resistant to at least one immunosuppressive agent due to the inactivation of a gene encoding a receptor for such immunosuppressive agent. In this aspect, the immunosuppressive treatment should help the selection and expansion of the T-cells according to the invention within the patient.

The present methods are more particularly designed for pre-treating patients eligible for bone marrow transplantation as part of so-called “bridge to transplant” medical strategies.

The administration of the cells or population of cells according to the present invention may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous or intralymphatic injection, or intraperitoneally. In one embodiment, the cell compositions of the present invention are preferably administered by intravenous injection.

The administration of the cells or population of cells can consist of the administration of 10⁴-10⁹ cells per kg body weight, preferably 10⁵ to 10⁶ cells/kg body weight including all integer values of cell numbers within those ranges. The present invention thus can provide more than 10, generally more than 50, more generally more than 100 and usually more than 1000 doses comprising between 10⁶ to 10⁸ gene edited cells originating from a single donor's or patient's sampling.

The cells or population of cells can be administrated in one or more doses. In another embodiment, said effective amount of cells are administrated as a single dose. In another embodiment, said effective amount of cells are administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions within the skill of the art. An effective amount means an amount which provides a therapeutic or prophylactic benefit. The dosage administrated will be dependent upon the age, health and weight of the recipient, kind of concurrent treatment, if any, frequency of treatment and the nature of the effect desired.

In another embodiment, said effective amount of cells or composition comprising those cells are administrated parenterally. Said administration can be an intravenous administration. Said administration can be directly done by injection within a tumor.

In certain embodiments of the present invention, cells are administered to a patient in conjunction with (e.g., before, simultaneously or following) any number of relevant treatment modalities, including but not limited to treatment with agents such as antiviral therapy, cidofovir and interleukin-2, Cytarabine (also known as ARA-C) or nataliziimab treatment for MS patients or efaliztimab treatment for psoriasis patients or other treatments for PML patients. In further embodiments, the T cells of the invention may be used in combination with chemotherapy, radiation, immunosuppressive agents, such as cyclosporin, azathioprine, methotrexate, mycophenolate, and FK506, antibodies, or other immune-ablative agents such as CAMPATH, anti-CD3 antibodies or other antibody therapies, cytoxin, fludaribine, cyclosporin, FK506, rapamycin, mycoplienolic acid, steroids, FR901228, cytokines, and irradiation. These drugs inhibit either the calcium dependent phosphatase calcineurin (cyclosporine and FK506) or inhibit the p70S6 kinase that is important for growth factor induced signaling (rapamycin) (Henderson, Naya et al. 1991; Liu, Albers et al. 1992; Bierer, Hollander et al. 1993). Ina further embodiment, the cell compositions of the present invention are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH, In another embodiment, the cell compositions of the present invention are administered following B-cell ablative therapy such as agents that react with CD20, e.g., Rituxan. For example, in one embodiment, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

### Combination Therapy Involving at Least Two Sub-Populations of T Cells.

The present invention encompasses methods and compositions combining engineered cells according to the invention exhibiting distinct features.

Accordingly, the present invention is also drawn to compositions of populations of primary TCR negative T-cells that can result from a single donor comprising at least two subpopulations of T-cells, said subpopulations comprising, for instance different gene edited immune checkpoint genes. Such sub-populations of cells can be selected, for instance, from:
TCR negative and PD1 negative,
TCR negative and CD52 negative,
TCR negative and CTLA4 negative,
TCR negative and dCK negative,
TCR negative and GR negative,
TCR negative and Um negative.

The engineered cells can be optionally transformed to express chimeric antigen receptor to provide allogeneic CAR T Cells directed to different surface molecules in order to reduce tumor escape, such as by combining for instance:
CAR CD123 and CAR CLL1,
CAR CD19 and CAR CD22,
CAR CD22 and CAR CD20,
CAR CD19 and CAR CD20,
CAR CS1 and CAR BCMA,
CAR CS1 and CAR CD38,
CAR BCMA and CAR CD38.

Also the engineered cells of the present invention may simultaneously or separately express IC engagers directed to different types of immune cells and target antigens, such as directing altogether CD3, CD16 and CD40 positive immune cells towards pathological targeted cells.

Such sub-populations can be used separately or in combination with each other into compositions for therapeutic treatments, in the same way as previously described with a single population of cells.

TABLE 12
LIST OF BISPECIFIC ANTIBODIES DIRECTED AGAINST HEMATOLOGIC MALIGNANCIES T-CELL ENGAGERS
(CD3 TARGETING)

| TARGET | PRODUCT EXAMPLES | Manufacturers | IC Engager type | PREFERRED INDICATION(S) |
|---|---|---|---|---|
| CD19/CD3 | Blinatumomab | Amgen | BITE | B-ALL, B-NHL, DLBCL, Richter |
| | | | | syndrome, MM |
| | Duvortuxizumab | MacroGenics | DART | CLL, B-NHL |
| | AFM11 | Affimed | TandAb | B-cell leukemia, B-NHL |
| CD20/CD3 | Glofitamab | Roche | CrossMab | B-cell lymphoma, B-NHL, DLBCL, FL |
| | Mosunetuzumab | Roche | DutaMab | B-cell lymphoma, B-NHL, B-NHL & CLL, |
| | | | | DLBCL, FL |
| | FBTA05 | Fresenius | TrioMab | B-cell lymphoma |
| | REGN1979 | Regeneron | Veloci-Bi | B-cell lymphoma, B-NHL, B-NHL & CLL |
| | Plamotamab | Xencor | Xmab | B-NHL, CLL & SLL |
| | CD20Bi-armed autologous | National Cancer Institute | AATC | B-cell lymphoma, MM & PCN |
| | T-cells | | | |
| CD33/CD3 | AMG330 and AMG564 | Amgen | BITE | AML |
| | GEM333 | GeMoAb & Celgene | ATAC | AML |
| CD123/CD3 | Flotetuzumab | MacroGenics | DART | AML, hematologic malignancies |
| | JNJ63709178 | Johnson & Johnson | DuoBody | AML |
| | Vibecotamab | Xencor | Xmab | AML, CML, BPDCN & B-ALL |
| | APVO436 | Aptevo Therapeutics | Adaptir | AML & MDS |
| CLL1/CD3 | MCLA117 | Merus | Biclonics | AML |
| CD38/CD3 | AMG424 | Amgen | BITE | MM |
| | GBR1342 | Glenmark | BEAT | MM |
| | | Pharmaceuticals | | |
| BCMA/CD3 | AMG420 | Amgen | BITE | MM |
| | Teclistamab | Johnson & Johnson | DuoBody | MM |
| | PF06863135 | Pfizer | Hinge mutations | MM |
| FCRH5/CD3 | BFCR4350A | Roche | BITE | MM |

TABLE 13
LIST OF BISPECIFIC ANTIBODIES DIRECTED AGAINST SOLID TUMORS T-CELL ENGAGERS (CD3 TARGETING)

| TARGET | PRODUCT EXAMPLES | Manufacturers | IC Engager type | PREFERRED INDICATION(S) |
|---|---|---|---|---|
| B7H3/CD3 | MGD009 | MacroGenics | DART | Solid tumors |
| CDH3/CD3 | PF06671008 | MacroGenics & Pfizer | DART | Solid tumors |
| CEA/CD3 | AMG111 | Amgen | BITE | Solid tumors |
| | AMG211 | | | Gastrointestinal adenocarcinoma |
| | RO6958688 | Roche | CrossMab | Solid tumors, NSCLC |
| DLL3/CD3 | AMG757 | Amgen | BITE | SCLC |
| EGFR/CD3 | EGFRBi-armed | National Cancer | AATC | Solid tumors, pancreatic adenocarcinoma, lung cancer |
| | autologous T-cells | Institute | | |
| EpCAM/CD3 | Solitomab | Amgen | BITE | Epithelial cancer, ovarian cancer, gastric |
| | | | | adenocarcinoma, malignant ascites, stomach neoplasms |
| | Catumaxomab | Fresenius & Trion | TrioMab | Epithelial cancer, ovarian cancer, gastric |
| | | Pharma | | adenocarcinoma, malignant ascites, stomach neoplasms |
| GD2/CD3 | GD2Bi-armed | National Cancer | AATC | Neuroblastoma |
| | autologous T-cells | Institute | | |
| GPA33/CD3 | MGD007 | MacroGenics | DART | Colorectal cancer |
| GPC3/CD3 | ERY974 | Chugai & Roche | TRAB | Solid tumors |
| HER2/CD3 | GBR1302 | Glenmark | BEAT | Solid tumors, breast cancer |
| | | Pharmaceuticals | | |
| | Ertumaxomab | Fresenius | Triomab | Solid tumors, breast cancer |
| PSCA/CD33 | GEM3PSCA | GeMoAb & Celgene | ATAC | NSCLC, breast, pancreatic and urogenital cancers |
| PSMA/CD3 | APVO414 | Aptevo Therapeutics | Adaptir | Prostate cancer |
| | Pasotuxizumab | Amgen | BITE | Prostate cancer |
| SSTR2/CD3 | Tidutamab | Xencor | XmAb | Neuroendocrine & gastrointestinal cancers |

TABLE 14
LIST OF BISPECIFIC ANTIBODIES DIRECTED AGAINST HEMATOLOGIC MALIGNANCIES NK CELL ENGAGERS
(CD16 TARGETING)

| | PRODUCT | | | |
|---|---|---|---|---|
| TARGET | EXAMPLES | Manufacturers | IC Engager type | PREFERRED INDICATION(S) |
| CD30/CD16A | AFM13 | Affimed | TandAb | HL, PTCL, ALCL |
| CD33/IL-15/CD16 | OXS-3550 | GT Biopharma | TriKe | AML & MDS |

TABLE 15
Genotype of some preferred engineered immune cells expressing IC engagers according to the present invention:

| | | Genotype of preferred engineereed Immune cells | | | |
|---|---|---|---|---|---|
| | | | | Optional KO to | |
| | | | Immune cell engagers | inactivate | Optional KO to confer |
| | | KO to lower alloreactivity: | Ex: | checkpoint inhibitors. | resistance to |
| | | Ex: | anti CD3 | Ex: [PD1] ^{neg} | immunodepletion agents. |
| CAR | | [TCR]^{neg} | anti CD40 | [CTLA4] ^{neg} | Ex: |
| | Preferred | [ß2m]^{neg} | anti-CD16, | or inflammatory pathways | glucocorticoids receptors: [GR] ^{neg} |
| CAR | Indication | [HLA]^{neg} | anti 4-1BB | Ex: [GM-CSF] ^{neg} | Alemtuzumab receptor [CD52] ^{neg} |
| [CAR CD19]^{pos} | Liquid | [CAR CD19] ^{pos} [TCR]^{neg} | [CAR CD19]^{pos}[TCR]^{neg} [IC CD3-CD19]^{pos} | +[PD1] ^{neg} | +[CD52] ^{neg} |
| [CAR CD20]^{pos} | tumors | [CAR CD19] ^{pos} [ß2m]^{neg} | [CAR CD19]^{pos}[ß2m]^{neg} [IC CD40-CD19]^{pos} | +[CTLA4] ^{neg} | +[GR] ^{neg} |
| [CAR CD22]^{pos} | | [CAR CD19] ^{pos} [HLA]^{neg} | [CAR CD19]^{pos}[HLA]^{neg} [IC CD16-CD19]^{pos} | +[TIM-3] ^{neg} | |
| | | [CAR CD19] ^{pos} [TCR]^{neg}[ß2m]^{neg} | [CAR CD19]^{pos}[TCR]^{neg} [ß2m]^{neg} [IC 4-1BB-CD19]^{pos} | +[LAG3] ^{neg} | |
| [CAR BCMA]^{pos} | | [CAR CD20] ^{pos} [TCR]^{neg} | [CAR CD19]^{pos}[TCR]^{neg} [IC CD3-CD20]^{pos} | +[TIGIT] ^{neg} | |
| [CAR CS1]^{pos} | | [CAR CD20] ^{pos} [ß2m]^{neg} | [CAR CD19]^{pos}[ß2m]^{neg} [IC CD40-CD20]^{pos} | +[GM-CSF] ^{neg} | |
| [CAR CD38]^{pos} | | [CAR CD20] ^{pos} [HLA]^{neg} | [CAR CD19]^{pos}[HLA]^{neg} [IC CD16-CD20]^{pos} | | |
| | | [CAR CD20] ^{pos} [TCR]^{neg}[ß2m]^{neg} | [CAR CD19]^{pos}[TCR]^{neg} [ß2m]^{neg} [IC 4-1BB-CD20]^{pos} | | |
| [CAR CD123]^{pos} | | [CAR CD22] ^{pos} [TCR]^{neg} | [CAR CD19]^{pos}[TCR]^{neg} [IC CD3-CD22]^{pos} | | |
| [CAR CLL1]^{pos} | | [CAR CD22] ^{pos} [ß2m]^{neg} | [CAR CD19]^{pos}[ß2m]^{neg} [IC CD40-CD22]^{pos} | | |
| | | [CAR CD22] ^{pos} [HLA] ^{neg} | [CAR CD19]^{pos}[HLA]^{neg} [IC CD16-CD22]^{pos} | | |
| | | [CAR CD22] ^{pos} [TCR]^{neg}[ß2m]^{neg} | [CAR CD19]^{pos}[TCR]^{neg} [ß2m]^{neg} [IC 4-1BB-CD22]^{pos} | | |
| | | [CAR BCMA] ^{pos} [TCR] ^{neg} | [CAR CD20]^{pos}[TCR]^{neg} [IC CD3-CD19]^{pos} | | |
| | | [CAR BCMA] ^{pos} [ß2m ]^{neg} | [CAR CD20]^{pos}[ß2m]^{neg} [IC CD40-CD19]^{pos} | | |
| | | [CAR BCMA] ^{pos} [HLA]^{neg} | [CAR CD20]^{pos}[HLA]^{neg} [IC CD16-CD19]^{pos} | | |
| | | [CAR BCMA] ^{pos} [TCR]^{neg}[ß2m]^{neg} | [CAR CD20]^{pos}[TCR]^{neg} [ß2m]^{neg} [IC 4-1BB-CD19]^{pos} | | |
| | | [CAR CS1] ^{pos} [TCR]^{neg} | [CAR CD20]^{pos}[TCR]^{neg} [IC CD3-CD20]^{pos} | | |
| | | [CAR CS1] ^{pos} [ß2m]^{neg} | [CAR CD20]^{pos}[ß2m]^{neg} [IC CD40-CD20]^{pos} | | |
| | | [CAR CS1] ^{pos} [HLA] ^{neg} | [CAR CD20]^{pos}[HLA]^{neg} [IC CD16-CD20]^{pos} | | |
| | | [CAR CS1] ^{pos} [TCR]^{neg}[ß2m]^{neg} | [CAR CD20]^{pos}[TCR]^{neg} [ß2m]^{neg} [IC 4-1BB-CD20]^{pos} | | |
| | | [CAR CD38] ^{pos} [TCR]^{neg} | [CAR CD20]^{pos}[TCR]^{neg} [IC CD3-CD22]^{pos} | | |
| | | [CAR CD38] ^{pos} [ß2m]^{neg} | [CAR CD20]^{pos}[ß2m]^{neg} [IC CD40-CD22]^{pos} | | |
| | | [CAR CD38] ^{pos} [HLA] ^{neg} | [CAR CD20]^{pos}[HLA]^{neg} [IC CD16-CD22]^{pos} | | |
| | | [CAR CD38] ^{neg} [TCR]^{neg}[ß2m]^{neg} | [CAR CD20]^{pos}[TCR]^{neg} [ß2m]^{neg} [IC 4-1BB-CD22]^{pos} | | |
| | | | [CAR CD22]^{pos}[TCR]^{neg} [IC CD3-CD19]^{pos} | | |
| | | | [CAR CD22]^{pos}[ß2m]^{neg} [IC CD40-CD19]^{pos} | | |
| | | | [CAR CD22]^{pos}[HLA]^{neg} [IC CD16-CD19]^{pos} | | |
| | | | [CAR CD22]^{pos}[TCR]^{neg} [ß2m]^{neg} [IC 4-1BB-CD19]^{pos} | | |
| | | | [CAR CD22]^{pos}[TCR]^{neg} [IC CD3-CD20]^{pos} | | |
| | | | [CAR CD22]^{pos}[ß2m]^{neg} [IC CD40-CD20]^{pos} | | |
| | | | [CAR CD22]^{pos}[HLA]^{neg} [IC CD16-CD20]^{pos} | | |
| | | | [CAR CD22]^{pos}[TCR]^{neg} [ß2m]^{neg} [IC 4-1BB-CD20]^{pos} | | |
| | | | [CAR CD22]^{pos}[TCR]^{neg} [IC CD3-CD22]^{pos} | | |
| | | | [CAR CD22]^{pos}[ß2m]^{neg} [IC CD40-CD22]^{pos} | | |
| | | | [CAR CD22]^{pos}[HLA]^{neg} [IC CD16-CD22]^{pos} | | |
| | | | [CAR CD22]^{pos}[TCR]^{neg} [ß2m]^{neg} [IC 4-1BB-CD22]^{pos} | | |
| | | | [CAR BCMA]^{pos} [TCR]^{neg} [IC CD3-BCMA]^{pos} | | |
| | | | [CAR BCMA]^{pos} [ß2m ]^{neg} [IC CD40-BCMA]^{pos} | | |
| | | | [CAR BCMA]^{pos} [HLA]^{neg} [IC CD16-BCMA]^{pos} | | |
| | | | [CAR BCMA]^{pos} [TCR]^{neg} [ß2m]^{neg} [IC 4-1BB-BCMA]^{pos} | | |
| | | | [CAR BCMA]^{pos} [TCR]^{neg} [IC CD3-CS1]^{pos} | | |
| | | | [CAR BCMA]^{pos} [ß2m ]^{neg} [IC CD40-CS1]^{pos} | | |
| | | | [CAR BCMA]^{pos} [HLA]^{neg} [IC CD16-CS1]^{pos} | | |
| | | | [CAR BCMA]^{pos} [TCR]^{neg} [ß2m]^{neg} [IC 4-1BB-CS1]^{pos} | | |
| | | | [CAR BCMA]^{pos} [TCR]^{neg} [IC CD3-CD38]^{pos} | | |
| | | | [CAR BCMA]^{pos} [ß2m ]^{neg} [IC CD40- CD38]^{pos} | | |
| | | | [CAR BCMA]^{pos} [HLA]^{neg} [IC CD16- CD38]^{pos} | | |
| | | | [CAR BCMA]^{pos} [TCR]^{neg}[ß2m]^{neg} [IC 4-1BB-CS1]^{pos} | | |
| | | | [CAR CS1]^{pos} [TCR]^{neg} [IC CD3-CS1]^{pos} | | |
| | | | [CAR CS1]^{pos} [ß2m]^{neg} [IC CD40-CS1]^{pos} | | |
| | | | [CAR CS1]^{pos} [HLA]^{neg} [IC CD16-CS1]^{pos} | | |
| | | | [CAR CS1]^{pos} [TCR]^{neg}[ß2m]^{neg} [IC 4-1BB-CS1]^{pos} | | |
| | | | [CAR CS1]^{pos} [TCR]^{neg} [IC CD3-BCMA]^{pos} | | |
| | | | [CAR CS1]^{pos} [ß2m]^{neg} [IC CD40-BCMA]^{pos} | | |
| | | | [CAR CS1]^{pos} [HLA]^{neg} [IC CD16-BCMA]^{pos} | | |
| | | | [CAR CS1]^{pos} [TCR]^{neg}[ß2m]^{neg} [IC 4-1BB-BCMA]^{pos} | | |
| | | | [CAR CS1]^{pos} [TCR]^{neg} [IC CD3-CD38]^{pos} | | |
| | | | [CAR CS1]^{pos} [ß2m]^{neg} [IC CD40-CD38]^{pos} | | |
| | | | [CAR CS1]^{pos} [HLA]^{neg} [IC CD16-CD38]^{pos} | | |
| | | | [CAR CS1]^{pos} [TCR]^{neg}[ß2m]^{neg} [IC 4-1BB-CD38]^{pos} | | |
| | | | [CAR CD38]^{pos} [TCR]^{neg} [IC CD3-CD38]^{pos} | | |
| | | | [CAR CD38]^{pos} [ß2m]^{neg} [IC CD40-CD38]^{pos} | | |
| | | | [CAR CD38]^{pos} [HLA]^{neg} [IC CD16-CD38]^{pos} | | |
| | | | [CAR CD38]^{pos} [TCR]^{neg}[ß2m]^{neg} [IC 4-1BB-CD38]^{pos} | | |
| | | | [CAR CD38]^{pos} [TCR]^{neg} [IC CD3-CS1]^{pos} | | |
| | | | [CAR CD38]^{pos} [ß2m]^{neg} [IC CD16-CS1]^{pos} | | |
| | | | [CAR CD38]^{pos} [HLA]^{neg} [IC CD40-CS1]^{pos} | | |
| | | | [CAR CD38]^{pos} [TCR]^{neg}[ß2m]^{neg} [IC 4-1BB-CS1]^{pos} | | |
| | | | [CAR CD38]^{pos} [TCR]^{neg} [CD3-BCMA]^{pos} | | |
| | | | [CAR CD38]^{pos} [ß2m]^{neg} [IC CD16-BCMA]^{pos} | | |
| | | | [CAR CD38]^{pos} [HLA]^{neg} [IC CD40-BCMA]^{pos} | | |
| | | | [CAR CD38]^{pos} [TCR]^{neg}[ß2m]^{neg} [IC 4-1BB-BCMA]^{pos} | | |
| | | | [CAR CLL1]^{pos} [TCR]^{neg} [IC CD3-CLL1]^{pos} | | |
| | | | [CAR CLL1]^{pos} [ß2m]^{neg} [IC CD16-CLL1]^{pos} | | |
| | | | [CAR CLL1]^{pos} [HLA]^{neg} [IC CD40-CLL1]^{pos} | | |
| | | | [CAR CLL1]^{pos} [TCR]^{neg}[ß2m]^{neg} [IC 4-1BB-CLL1]^{pos} | | |
| | | | [CAR CLL1]^{pos} [TCR]^{neg} [CD3-CD123]^{pos} | | |
| | | | [CAR CLL1]^{pos} [ß2m]^{neg} [IC CD16-CD123]^{pos} | | |
| | | | [CAR CLL1]^{pos} [HLA]^{neg} [IC CD40-CD123]^{pos} | | |
| | | | [CAR CLL1]^{pos} [TCR]^{neg}[ß2m]^{neg} [IC 4-1BB-CD123]^{pos} | | |
| | | | [CAR CD123]^{pos} [TCR]^{neg} [IC CD3-CLL1]^{pos} | | |
| | | | [CAR CD123]^{pos} [ß2m]^{neg} [IC CD16-CLL1]^{pos} | | |
| | | | [CAR CD123]^{pos} [HLA]^{neg} [IC CD40-CLL1]^{pos} | | |
| | | | [CAR CD123]^{pos} [TCR]^{neg}[ß2m]^{neg} [IC 4-1BB-CLL1]^{pos} | | |
| | | | [CAR CD123]^{pos} [TCR]^{neg} [CD3-CD123]^{pos} | | |
| | | | [CAR CD123]^{pos} [ß2m]^{neg} [IC CD16-CD123]^{pos} | | |
| | | | [CAR CD123]^{pos} [HLA]^{neg} [IC CD40-CD123]^{pos} | | |
| | | | [CAR CD123]^{pos} [TCR]^{neg}[ß2m]^{neg} [IC 4-1BB-CD123]^{pos} | | |
| [CAR TROP2]^{pos} | Solid | [CAR TROP2]^{pos}[TCR]^{neg} | [CAR TROP2]^{pos} [TCR]^{neg} [IC CD3-TROP2]^{pos} | +[PD1] ^{neg} | +[CD52] ^{neg} |
| [CAR MUC1]^{pos} | tumors | [CAR TROP2]^{pos}[ß2m]^{neg} | [CAR TROP2]^{pos} [ß2m]^{neg} [IC CD40-TROP2]^{pos} | +[CTLA4] ^{neg} | +[GR] ^{neg} |
| [CAR MSLN]^{pos} | | [CAR TROP2]^{pos}[HLA]^{neg} | [CAR TROP2]^{pos} [HLA]^{neg} [IC CD16-TROP2]^{pos} | +[TIM-3] ^{neg} | |
| | | [CAR TROP2]^{pos}[TCR]^{neg}[ß2m]^{neg} | [CAR TROP2]^{pos} [TCR]^{neg}[ß2m]^{neg} [IC 4-1BB-TROP2]^{pos} | +[LAG3] ^{neg} | |
| | | [CAR MUC1]^{pos}[TCR]^{neg} | [CAR TROP2]^{pos} [TCR]^{neg} [IC CD3-MUC1]^{pos} | +[TIGIT] ^{neg} | |
| | | [CAR MUC1]^{pos}[ß2m]^{neg} | [CAR TROP2]^{pos} [ß2m]^{neg} [IC CD40-MUC1]^{pos} | +[GM-CSF] ^{neg} | |
| | | [CAR MUC1]^{pos}[HLA]^{neg} | [CAR TROP2]^{pos} [HLA]^{neg} [IC CD16-MUC1]^{pos} | | |
| | | [CAR MUC1]^{pos}[TCR]^{neg}[ß2m]^{neg} | [CAR TROP2]^{pos} [TCR]^{neg}[ß2m]^{neg} [IC 4-1BB-MUC1]^{pos} | | |
| | | [CAR MSLN]^{pos}[TCR]^{neg} | [CAR TROP2]^{pos} [TCR]^{neg} [IC CD3-MSLN]^{pos} | | |
| | | [CAR MSLN]^{pos}[ß2m]^{neg} | [CAR TROP2]^{pos} [ß2m]^{neg} [IC CD40-MSLN]^{pos} | | |
| | | [CAR MSLN]^{pos}[HLA]^{neg} | [CAR TROP2]^{pos} [HLA]^{neg} [IC CD16-MSLN]^{pos} | | |
| | | [CAR MSLN]^{pos}[TCR]^{neg}[ß2m]^{neg} | [CAR TROP2]^{pos} [TCR]^{neg}[ß2m]^{neg} [IC 4-1BB-MSLN]^{pos} | | |
| | | | [CAR MUC1]^{pos} [TCR]^{neg} [IC CD3-TROP2]^{pos} | | |
| | | | [CAR MUC1 ]^{pos} [ß2m]^{neg} [IC CD40-TROP2]^{pos} | | |
| | | | [CAR MUC1]^{pos} [HLA]^{neg} [IC CD16-TROP2]^{pos} | | |
| | | | [CAR MUC1]^{pos} [TCR]^{neg}[ß2m]^{neg} [IC 4-1BB-TROP2]^{pos} | | |
| | | | [CAR MUC1]^{pos} [TCR]^{neg} [IC CD3-MUC1]^{pos} | | |
| | | | [CAR MUC1]^{pos} [ß2m]^{neg} [IC CD40-MUC1]^{pos} | | |
| | | | [CAR MUC1]^{pos} [HLA]^{neg} [IC CD16-MUC1]^{pos} | | |
| | | | [CAR MUC1]^{pos} [TCR]^{neg}[ß2m]^{neg} [IC 4-1BB-MUC1]^{pos} | | |
| | | | [CAR MUC1]^{pos} [TCR]^{neg} [IC CD3-MSLN]^{pos} | | |
| | | | [CAR MUC1]^{pos} [ß2m]^{neg} [IC CD40-MSLN]^{pos} | | |
| | | | [CAR MUC1]^{pos} [HLA]^{neg} [IC CD16-MSLN]^{pos} | | |
| | | | [CAR MUC1]^{pos} [TCR]^{neg}[ß2m]^{neg} [IC 4-1BB-MSLN]^{pos} | | |
| | | | [CAR MSLN]^{pos} [TCR]^{neg} [IC CD3-TROP2]^{pos} | | |
| | | | [CAR MSLN]^{pos} [ß2m]^{neg} [IC CD40-TROP2]^{pos} | | |
| | | | [CAR MSLN]^{pos} [HLA]^{neg} [IC CD16-TROP2]^{pos} | | |
| | | | [CAR MSLN]^{pos} [TCR]^{neg}[ß2m]^{neg} [IC 4-1BB-TROP2]^{pos} | | |
| | | | [CAR MSLN]^{pos} [TCR]^{neg} [IC CD3-MUC1]^{pos} | | |
| | | | [CAR MSLN]^{pos} [ß2m]^{neg} [IC CD40-MUC1]^{pos} | | |
| | | | [CAR MSLN]^{pos} [HLA]^{neg} [IC CD16-MUC1]^{pos} | | |
| | | | [CAR MSLN]^{pos} [TCR]^{neg}[ß2m]^{neg} [IC 4-1BB-MUC1]^{pos} | | |
| | | | [CAR MSLN]^{pos} [TCR]^{neg} [IC CD3-MSLN]^{pos} | | |
| | | | [CAR MSLN]^{pos} [ß2m]^{neg} [IC CD40-MSLN]^{pos} | | |
| | | | [CAR MSLN]^{pos} [HLA]^{neg} [IC CD16-MSLN]^{pos} | | |
| | | | [CAR MSLN]^{pos} [TCR]^{neg}[ß2m]^{neg} [IC 4-1BB-MSLN]^{pos} | | |

The above written description of the invention provides a manner and process of making and using it such that any person skilled in this art is enabled to make and use the same, this enablement being provided in particular for the subject matter of the appended claims, which make up a part of the original description.

Where a numerical limit or range is stated herein, the endpoints are included. Also, all values and subranges within a numerical limit or range are specifically included as if explicitly written out.

Having generally described this invention, a further understanding can be obtained by reference to certain specific examples, which are provided herein for purposes of illustration only, and are not intended to limit the scope of the claimed invention.

### EXAMPLES

### Example 1: Production of Anti-CD123 CAR-T Expressing CLL1 BiTE ([CAR CD123]^{pos} [TCR]^{neg} [IC CD3-CLL1]^{pos})

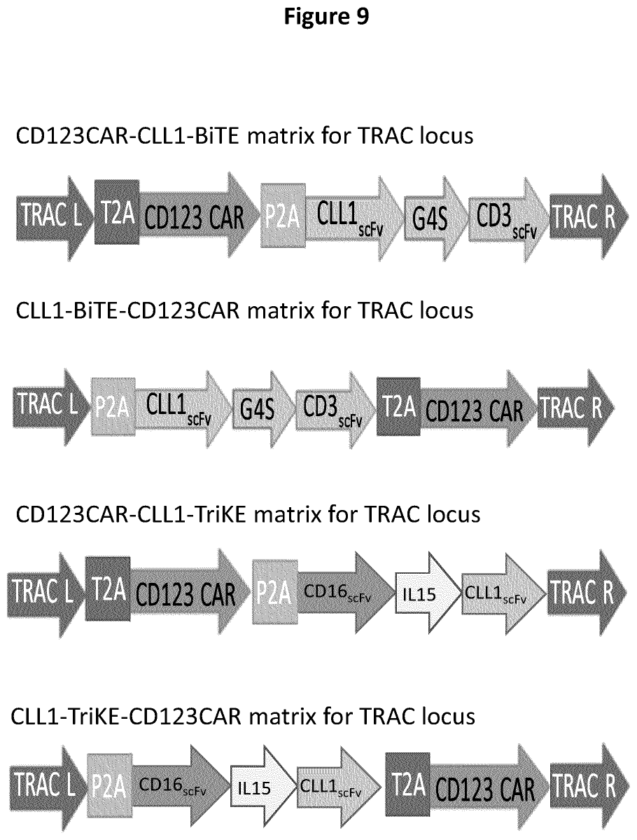

Cryopreserved PBMC were thawed at 37° C., washed and re-suspended in OpTmizer medium supplemented with AB human serum (5%) for overnight incubation at 37° C. in 5% CO₂ incubator. Cells were then activated with antiCD3/CD28 coated beads in OpTmizer medium supplemented with AB human serum (5%) and recombinant human interleukin-2 (rhIL-2, 350 IU/mL) in a CO₂ incubator (culture medium). Three days after activation the amplified T-cells were electroporated with the 5 μg of mRNAs encoding TRAC TALEN arms (SEQ ID NO. 23 and SEQ ID NO. 24). Transfection was performed using Pulse Agile technology by applying two 0.1 mS pulses at 800V followed by four 0.2 mS pulses at 130 V in 0.4 cm gap cuvettes in Cytoporation buffer T (BTX Harvard Apparatus, Holliston, Mass.). The electroporated cells were then immediately transferred into prewarmed Optmizer serum-free media and incubated at 37° C. for 15 min and for another 15 min at 30° C. The cells were then concentrated and incubated in the presence of AAV6 particles (MOI=1E5 vg/cells) comprising one of the donor matrixes depicted in FIG. 9. These donor matrixes are composed of 300 bp of the TRAC left and right Homology arms, a self-cleaving T2A peptide (SEQ ID NO: 33) allowing the expression of the CD123CAR (SEQ ID NO: 13), a self-cleaving P2A peptide (SEQ ID NO: 34) allowing the expression and secretion of a CLL1 BiTE (SEQ ID NO: 38). After 2 h of culture at 30° C., Optmizer media supplemented by 10% AB serum and IL-2 was added to the cell suspension, and the mix was incubated for 16 h under the same culture conditions. Cells were then cultivated at 37° C. in the presence of 5% CO₂. On the final day of culture TCRαβ negative cells were isolated, resuspended in culture medium for an over-night culture. The next day cells were either used directly used or kept frozen in freezing medium (10% DMSO in FBS) until use. The sequence used in these experiments are reported in Tables 1 and 2.

### Example 2: CLL1 BiTEs from CD123CAR T Redirect PBMC Towards Tumor Cells

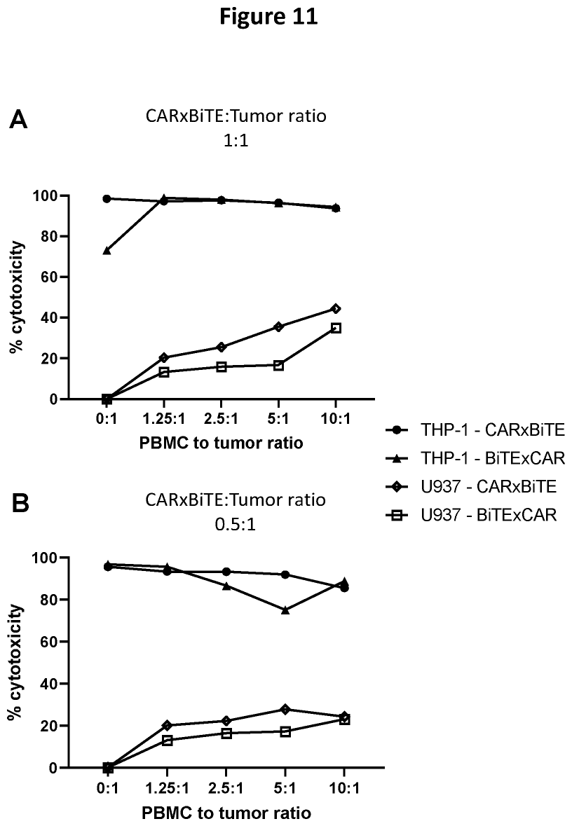

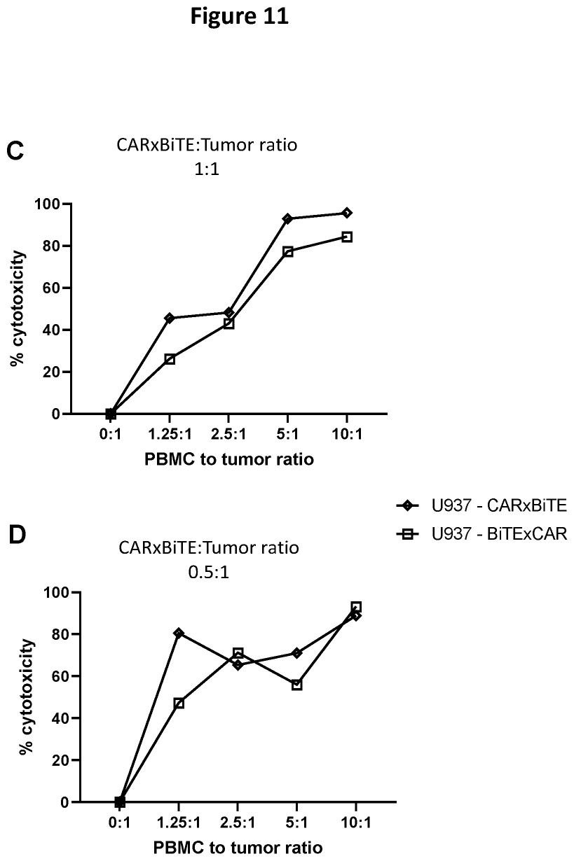

Tumor cell lines THP-1 (CD123 positive and CLL1 positive cells) and U937 (CLL1 positive and CD123 negative cells) were labeled with CellTrace Violet dye (0.5 mM). These target cells were co-cultured with i) either mock-transduced T cells or CD123CAR-2A-CLL1-BiTE (SEQ ID NO:39) or CLL1-BiTE-2A-CD123CAR (SEQ ID NO:40) at effector to target ratios of 0.5:1 and 1:1 and ii) in the presence (or absence) of thawed cryopreserved human PBMCs (ALLCELLS) at different PBMC:tumor ratios. The cell mix was incubated in a 96-well plate for 24 hrs and 48 hrs at 37° C., 5% CO₂. Cells were cultured in complete medium RPMI 1640 supplemented with either 10% heat-inactivated FBS and 0.05 mM 2-mercaptoethanol or 1% Pen/Strep for THP-1 and U937, respectively. After 24 or 48 hours incubation, cells were stained with Fixable Viability Dye eFluor 780 (20 μL/well at 1:1000 dilution) for analysis by flow cytometry. The results shown in FIG. 11 represent the percent cytotoxicity of tumor cells in the presence of effector cells normalized to that in the absence of effector cells after normalized to percent cytotoxicity in the presence of mock-transfected cells obtained after 24 (11A and 11B) or 48 hours (11C and 11D) incubation respectively. For THP-1 cell line, the presence of CD123CAR T led to the total killing of this cell line whatever condition with PBMC In absence of PBMC, U937 were spared by CD123CAR, but the cytotoxicity of U937 was increasing with the increasing amount of PBMC and led to a total killing of U937 after a 48 hours incubation (FIGS. 11C and 11D). These results demonstrate that the BiTE expressed and secreted by CD123CAR T cells were redirecting the PBMC to the U937 for an efficient killing.

### Example 3: Production of Anti-CD22 CAR-T Cells Expressing Blinatumomab Upon Activation ([TRAC]^{neg} [CAR CD22]^{pos}[GM-CSF]^{neg} [IC CD3-CD19]^{pos})

### Cells

Cryopreserved human PBMCs were acquired from ALLCELLS (catalog no. PB006F), and human monocytes were acquired from STEMCELL Technologies (catalog no. 70035.1). Both PBMCs and monocytes were cultured in X-vivo-15 media (Lonza, catalog no. BE04-418Q), containing IL-2 (Miltenyi Biotec, catalog no. 130-097-748) and human serum AB (Seralab, catalog no. GEM-100-318). Raji CD22 WT, Raji CD22 KO, and Daudi cells were cultured in RPMI 1640 media supplemented with 10% v/v FBS (Gibco, catalog no. 10437036), 100 units/ml penicillin and 100 μg/ml streptomycin.

### Antibodies and Reagents

Human T-activator CD3/CD28 (Life Technologies, Inc., catalog no. 11132D) was used to activate T-cells. CAR T-cells were stained using CD34 antibody QBEND10-APC (R&D Systems, catalog no. FAB7227A). Monocyte phenotyping was performed using antibodies against human CD14, CD11 b, and CD16 from Miltenyi Biotec (catalog nos. 130-110-524, 130-110-552, and 130-113-389, respectively). GMCSF neutralization antibody was purchased from R&D Systems (catalog no. MAB215). Human recombinant proteins GMCSF, IL-8, and TNFα were purchased from R&D Systems (catalog nos. 215-GM and 208-IL-010) and PeproTech (catalog no. 50-813-404), respectively. Human ELISA kits for GMCSF, IFNγ, IL-6, and TNFα were obtained from R&D Systems (catalog nos. DGM00, DIF50, H600C, and DTA00D, respectively). LEGENDplex cytokine assays (13-plex), with human inflammation panels 1 and 2, were obtained from the BioLegend (catalog nos. 740118 and 740775, respectively).

### Targeted Integration of Anti-CD22 CAR and Blinatumomab at the TRAC and GM-SCF Loci, Respectively in Primary T-Cells

The targeted integration of the anti-CD22 CAR transgene construct was performed by homologous recombination at the locus encoding TCR-alpha constant chain (TRAC). The targeted integration of the Blinatumomab transgene construct was performed by homologous recombination at the locus encoding Granulocyte-macrophage colony-stimulating factor (GM-CSF) [Uniprot: #P04141] in view of inactivating its expression at least partially. PBMC cells were first thawed, washed, resuspended, and cultivated in X-vivo-15 complete media (X-vivo-15, 5% AB v/v serum, 20 ng/ml IL-2). One day later, the cells were activated with the Dynabeads® human T activator CD3/CD28 (25 μl of beads/1E6 CD3 positive cells) and cultivated at a density of 1E6 cells/ml for 3 days in X-vivo complete media at 37° C. in the presence of 5% CO2. The cells were then passaged to 1E6 cells/ml in fresh complete media and transduced/transfected the next day according to the following procedure. On the day of transduction-transfection, the cells were first de-beaded by magnetic separation (EasySep), washed twice in Cytoporation buffer T (BTX Harvard Apparatus, Holliston, Mass.), and resuspended at a final concentration of 28E6 cells/ml in the same solution. 180 μl of the cell suspension (i.e. 5E6 cells) was mixed with 5 μg of mRNA encoding TRAC TALEN and 5 μg of mRNA encoding GMCSF TALEN (see Table 2 and 16 for target sequences—Left and right binding sites are indicated in uppercase, and spacers are indicated in lowercase) in a final volume of 200 μl. Transfection was performed using Pulse Agile Technology (BTX Harvard Apparatus). The electroporated cells were immediately transferred to a 12-well plate containing 1 ml of prewarmed X-vivo-15 serum-free media and incubated for 37° C. for 15 min. The cells were then concentrated to 8E6 cells/ml in 250 μl of the same media in the presence of AAV6 particles (multiplicity of infection=3E5 vg/cells) comprising the donor matrices in 48-well regular-treated plates. After 2 h of culture at 30° C., 250 μl of Xvivo-15 media supplemented by 10% AB serum and 40 ng/ml IL-2 was added to the cell suspension, and the mix was incubated overnight in the same culture conditions. On the next day, the cells were seeded at 1E6 cells/ml in complete X-vivo-15 media and cultivated at 37° C. in the presence of 5% CO2.

TABLE 16
TALEN target sequences used in Example 3
for CAR-Blinaturumab transgene
integration at the GM-CSF locus

| | SEQ | Sequence targeted in |
|---|---|---|
| TALEN | ID # | the primary T-cells |
| GMCSF_Tal_1 | 54 | TGTGGCTGCAGAGCCTGctgc |
| | | tcttgggCACTGTGGCCTGCA |
| | | GCA |
| GMCSF_Tal_2 | 55 | TCCTGAACCTGAGTAGAgaca |
| | | ctgctgcTGAGATGGTAAGTG |
| | | AGA |

### Transwell Assay

Transwell assays were performed using anti-CD22 CAR T-cells (GMCSF WT or KO) from multiple donors, co-cultured with tumor cells (bottom chamber) and human CD14+ monocytes (top chamber), and separated by a polystyrene membrane with a pore size of 0.4 μm. Briefly, 1E5 CAR T-cells and 5E4 tumor cells were incubated with 1E5 monocytes for various time points in the absence or presence of GMCSF antibody at increasing concentrations. The supernatant was collected after 16 h, unless stated otherwise, to measure cytokines using a BioLegend Human Inflammation 13-plex kit or ELISA. The CD14+ human monocytes used in this assay were acquired from STEMCELL Technologies. Approximately 1 h prior to the experiment, the cells were thawed at 37° C. in a water bath, and after centrifugation at 300×g for 5 min, the cells were resuspended and counted. For the transwell experiment, the cells were suspended in X-vivo media supplemented with 5% v/v human AB serum, the same media used for CAR T-cells suspension. This quick transition (˜1 h) between thawing and starting the experiment prevented any differentiation of monocytes into any other lineages.

### Serial Killing Assay

To assess the antitumor activity of the engineered CAR T-cells, a serial killing assay was performed according to Valton, J., et al. [A versatile safeguard for chimeric antigen receptor T-cell immunotherapies (2018) Sci. Rep. 8, 8972], by using a suspension of 2.5E5 Raji-luc tumor cells mixed with CAR T-cells at variable DT ratios (5:1, 3.5:1, 2.5:1, and 1:1) in a total volume of 1 ml of Xvivo media supplemented with 5% AB serum.

### Statistical Analysis

Statistical analysis was performed using Prism 6 (GraphPad Software) using either one-way or two-way ANOVA for comparisons wherever appropriate. p value significance was calculated using post-test Bonferroni or Dunnett's multiple comparisons test.

### CONCLUSION

Primary T-cells presenting the phenotype [TRAC]^{neg}[CAR CD22]^{pos}[GM-CSF]^{neg}[IC CD3-CD19]^{pos} can be successfully engineered by using anti-CD22 CAR expression cassette, co-transfection of (1) TRAC TALEN mRNAs and (2) GM-CSF TALEN mRNAs, and (co-) transduction of (3) AAV6 polynucleotide matrice comprising sequence encoding anti-CD22 CAR (SEQ ID NO:22), and (4) AAV6 polynucleotide matrice comprising sequence encoding Blinatumomab. We observed no differences in CAR expression among different groups of donors. GMCSF KO resulted in a 90% reduction in GMCSF secretion by CAR T-cells after 16 h of co-incubation with tumor cells. To confirm that GMCSF KO did not impair the proliferation and anti-tumor function of CAR T-cells, a tumor-mediated proliferation assay was performed and also a 24-h anti-tumor assay. No change was observed in either the proliferation capacity or anti-tumor properties of CAR T-cells after GMCSF KO in four independent donors treated with two different GMCSF TALEN constructs, suggesting that GMCSF KO does not impair the normal functions of CAR T-cells. A serial killing assay to challenge GMCSF KO CAR T-cells was performed with daily doses of tumor cells for six consecutive days. This assay showed similar results, with no impaired activity of GMCSF KO CAR T-cells compared with GMCSF wildtype (WT) cells performed at different effector to target (E/T) cell ratios. Finally, no difference was observed in the expansion of GMCSF KO CD4 CAR T-cells and GMCSF KO CD8 CAR T-cells.

Since GMCSF KO CAR T-cells proliferate as well as GMCSF WT CAR T-cells and exhibit similar anti-tumor properties, we then subjected these cells to the transwell assay described above. GMCSF KO CAR T-cells show suppressed secretion of inflammatory cytokines by monocytes. Consistent with activity tests, GMCSF KO do not impair the production of key CAR T-cell cytokines such as IFNγ. In addition, significant reduction in IL-6 and MCP produced by monocytes. GMCSF KO also led to a decrease in TNFα, and no change in IL-8 compared with CAR T-cells with WT GM-CSF. These results are consistent with a previous study by Sterner et al. [GM-CSF inhibition reduces cytokine release syndrome and neuroinflammation but enhances CAR-T cell function in xenografts (2019) Blood 133, 697-709], showing that knocking out GMCSF in anti-CD19 CAR T-cells by using CRISPR prevented cytokine release syndrome (CRS) symptoms such as weight loss and encephalopathy in a primary ALL xenograft. Similarly, the present invention points toward a strategy that could be used to prevent the side effects of CAR T-cell therapy.

In addition to anti-CD22 activity, the engineered CAR T-cells expressing Blinatumomab have a prolonged and increased activity in the transwell assay against tumor cells expressing CD19 and CD22 positive markers or tumor cells expressing CD19 that have lost CD22 expression. This improved activity is linked to Blinatumomab expression that redirects endogenous cells (from the patient) towards CD19 positive cells and limits rejection of allogenic CAR T-cells. Beside this effect, the fact that this allogeneic setting addresses both CD19 and CD22 positive cells, reduces tumor escape phenomenon.

In conclusion, we describe a strategy to engineer safer “all-in-one” CAR T-cells that confer lesser cytokine-mediated toxicity and extended activity, especially in allogeneic settings.

1. A method for producing therapeutic T-cells for use in allogeneic transfer settings, said method comprising:
(i) Providing a population of genetically engineered T-cells originating from a donor, in which expression of T-cell receptor is inhibited or inactivated;
(ii) Expressing in said population of T-cells at least one exogenous polynucleotide encoding a soluble immune cell engager (IC engager), wherein said immune cell engager is specifically directed against at least one patient's immune cell type.
(iii) Optionally, isolating T-cells that do not express TCR alpha at their cell surface.

2. Method according to claim 1, wherein the population of genetically engineered T-cells are derived from [CD34]+ hematopoietic pluripotent cells.

3. Method according to claim 1, wherein the population of genetically engineered T-cells are derived from induced pluripotent stem cells (iPS), Embryonic Stem Cells (ES) or umbilical stem cells.

4. Method according to claim 1, wherein the population of genetically engineered T-cells are derived from PBMC.

5. Method according to any one of claims 1 to 4, wherein at least 50%, preferably at least 70%, pref. at least 90%, more pref. at least 95% of said engineered T-cells in step i) express a short hairpin RNA (shRNA) or small interfering (siRNA) directed against a polynucleotide sequence encoding a component of TCR.

6. Method according to any one of claims 1 to 4, wherein at least 50%, preferably at least 70%, pref. at least 90%, more pref. at least 95% of said engineered T-cells in step i) are mutated in their TCRA and/or TCRB alleles.

7. Method according to claim 6, wherein said mutations in said TCRA or TCRB have been introduced by using rare-cutting endonucleases or base editors.

8. Method according to claim 7, wherein, wherein said rare-cutting endonuclease is a homing endonuclease, a TALE-Nuclease, a Mega-TAL-nuclease, a Zing Finger Nuclease or a RNA-guided endonuclease, such as CAS9 or CAS12 (CPF1).

9. Method according to any one of claims 7 and 8, wherein said nuclease cleaves a gene selected from CD3, TCRalpha, and/or TCRbeta.

10. Method according to any one of claims 1 to 9, wherein said soluble immune cell engager is directed against immune cells selected from T-cells, NK-cells, macrophages or antigen presenting cells (APC).

11. Method according to any one of claims 1 to 10, wherein said soluble immune cell engager binds an immune cell's activating receptor complex.

12. Method according to claim 11, wherein said soluble immune cell engager binds a component of the TCR.

13. Method according to claim 12, wherein said component of the T-cell receptor is selected from CD3, TCRalpha, TCRbeta, TCRgamma and/or TCR delta.

14. Method according to claim 13, wherein said soluble immune cell engager binds CD3 antigen.

15. Method according to claim 11, wherein said soluble immune cell engager is directed against NK cells.

16. Method according to claim 15, wherein said soluble immune cell engager binds CD16 surface antigen.

17. Method according to claim 11, wherein said soluble immune cell engager is directed against APC/macrophage.

18. Method according to claim 17, wherein said soluble immune cell engager binds CD40 surface antigen.

19. Method according to any one of claims 1 to 18, wherein said soluble immune cell engager is selected from bispecific T-cell engagers (BITE), dual-affinity re-targeting antibodies (DART), bispecific engagement by antibodies based on the T-cell receptor (BEAT), CROSSMAB, TRIOMAB, tandem diabody (TANDAB), ADAPTIR, affinity-tailored adaptors for T-cells (ATAC), DUOBODY, XMAB, T-cell redirecting antibody (TRAB), BICLONICS, DUTAMAB, VELOCI-BI, hinge-mutated, bispecific antibody-armed activated T-cells (AATC), bi- & tri-specific killer cell engagers (BIKE, TRIKE)

20. Method according to any one of claims 1 to 19, wherein said immune cell engager is a BiTE.

21. Method according to claim 19, wherein said IC engager binds CD3 and CD19.

22. Method according to claim 20, wherein said IC engager is a BiTE having a polypeptide sequence comprising SEQ ID NO. 42.

23. Method according to any one of claims 1 to 19, wherein said IC engager binds CD3 and CD22.

24. Method according to any one of claims 1 to 19, wherein said IC engager binds CD3 and CD20.

25. Method according to any one of claims 1 to 19, wherein said IC engager binds CD3 and CD123.

26. Method according to any one of claims 1 to 19, wherein said IC engager binds CD3 and CLL1.

27. Method according to any one of claims 1 to 19, wherein said IC engager binds CD3 and Mesothelin.

28. Method according to any one of claims 1 to 19, wherein said IC engager binds CD3 and Trop2.

29. Method according to any one of claims 1 to 19, wherein said IC engager binds CD16 and CD19.

30. Method according to any one of claims 1 to 19, wherein said IC engager binds CD16 and CD22.

31. Method according to any one of claims 1 to 19, wherein said IC engager binds CD16 and CD20.

32. Method according to any one of claims 1 to 19, wherein said IC engager binds CD16 and CD123.

33. Method according to any one of claims 1 to 19, wherein said IC engager binds CD16 and CLL1.

34. Method according to any one of claims 1 to 19, wherein said IC engager binds CD16 and Mesothelin.

35. Method according to any one of claims 1 to 19, wherein said IC engager binds CD16 and Trop2.

36. Method according to any one of claims 1 to 19, wherein said IC engager binds CD40 and CD19.

37. Method according to any one of claims 1 to 19, wherein said IC engager binds CD40 and CD22.

38. Method according to any one of claims 1 to 19, wherein said IC engager binds CD40 and CD20.

39. Method according to any one of claims 1 to 19, wherein said IC engager binds CD40 and CD123.

40. Method according to any one of claims 1 to 19, wherein said IC engager binds CD40 and CLL1.

41. Method according to any one of claims 1 to 19, wherein said IC engager binds CD40 and Mesothelin.

42. Method according to any one of claims 1 to 19, wherein said IC engager binds CD40 and Trop2.

43. Method according to any one of claims 1 to 42, wherein the polynucleotide sequence for expression of said immune cell engager has been introduced at a locus regulated by or encoding TCR, HLA, β2m, PD1, CTLA4, TIM3, GM-CSF, LAG3 CD69, IL2Ra, and/or CD52.

44. Method according to any one of claims 1 to 43, wherein the polynucleotide sequence for expression of said immune cell engager is introduced by using a viral vector.

45. Method according to any one of claims, wherein said immune cell engager further specifically binds at least one infection associated antigen or tumor antigen.

46. Method according to claim 45, wherein said tumor antigen is selected from: Interleukin 3 receptor subunit alpha.spanning 4-domains A1 (MS4A1 also known as CD20); CD22 molecule (CD22); CD229 molecule (CD229) CD24 molecule (CD24); CD248 molecule (CD248); CD276 molecule (CD276 or B7H3); CD3 molecule (CD3); CD33 molecule (CD33); CD38 molecule (CD38); CD44v6; CD5 molecule (CD5); CD56 molecule (CD56); CD7 molecule (CD7); CD70 molecule (CD70); CD72; CD79a; CD79b; TNF receptor superfamily member 8 (TNFRSF8 also known as CD30); KIT proto-oncogene receptor tyrosine kinase (CD117); V-set pre-B cell surrogate light chain 1 (VPREB1 or CD179a); adhesion G protein-coupled receptor E5 (ADGRE5 or CD97); TNF receptor superfamily member 17 (TNFRSF17 also known as BCMA); SLAM family member 7 (SLAMF7 also known as CS1); L1 cell adhesion molecule (L1CAM); C-type lectin domain family 12 member A (CLEC12A also known as CLL-1); tumor-specific variant of the epidermal growth factor receptor (EGFRvIII); thyroid stimulating hormone receptor (TSHR); Fms related tyrosine kinase 3 (FLT3); ganglioside GD3 (GD3); Tn antigen (Tn Ag); lymphocyte antigen 6 family member G6D (LY6G6D); Delta like canonical Notch ligand 3 (DLL3); Interleukin-13 receptor subunit alpha-2 (IL-13RA2); Interleukin 11 receptor subunit alpha (IL11RA); mesothelin (MSLN); Receptor tyrosine kinase like orphan receptor 1 (ROR1); Prostate stem cell antigen (PSCA); erb-b2 receptor tyrosine kinase 2 (ERBB2 or Her2/neu); Protease Serine 21 (PRSS21); Kinase insert domain receptor (KDR also known as VEGFR2); Lewis y antigen (LewisY); Solute carrier family 39 member 6 (SLC39A6); Fibroblast activation protein alpha (FAP); Hsp70 family chaperone (HSP70); Platelet-derived growth factor receptor beta (PDGFR-beta); Cholinergic receptor nicotinic alpha 2 subunit (CHRNA2); Stage-Specific Embryonic Antigen-4 (SSEA-4); Mucin 1, cell surface associated (MUC1); mucin 16, cell surface associated (MUC16); claudin 18 (CLDN18); claudin 6 (CLDN6); Epidermal Growth Factor Receptor (EGFR); Preferentially expressed antigen in melanoma (PRAME); Neural Cell Adhesion Molecule (NCAM); ADAM metallopeptidase domain 10 (ADAM10); Folate receptor 1 (FOLR1); Folate receptor beta (FOLR2); Carbonic Anhydrase IX (CA9); Proteasome subunit beta 9 (PSMB9 or LMP2); Ephrin receptor A2 (EphA2); Tetraspanin 10 (TSPAN10); Fucosyl GM1 (Fuc-GM1); sialyl Lewis adhesion molecule (sLe); TGS5; high molecular weight-melanoma-associated antigen (HMWMAA); o-acetyl-GD2 ganglioside (OAcGD2); tumor endothelial marker 7-related (TEM7R); G protein-coupled receptor class C group 5, member D (GPRC5D); chromosome X open reading frame 61 (CXORF61); ALK receptor tyrosine kinase (ALK); Polysialic acid; Placenta-specific 1 (PLAC1); hexasaccharide portion of globoH glycoceramide (GloboH); NY-BR-1 antigen; uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 family member K (LY6K); olfactory receptor family 51 subfamily E member 2 (OR51E2); TCR Gamma Alternate Reading Frame Protein (TARP); Wilms tumor protein (WT1); ETV6-AML1 fusion protein due to 12; 21 chromosomal translocation (ETV6-AML1); sperm autoantigenic protein 17 (SPA17); X Antigen Family, Member 1E (XAGE1E); TEK receptor tyrosine kinase (Tie2); melanoma cancer testis antigen-1 (MAD-CT-1); melanoma cancer testis antigen-2 (MAD-CT-2); Fos-related antigen 1; p53 mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B 1; v-myc avian myelocytomatosis viral oncogene neuroblastoma derived homolog (MYCN); Ras Homolog Family Member C (RhoC); Cytochrome P450 1B 1 (CYP1B 1); CCCTC-Binding Factor (Zinc Finger Protein)-Like (BORIS); Squamous Cell Carcinoma Antigen Recognized By T Cells 3 (SART3); Paired box protein Pax-5 (PAX5); proacrosin binding protein sp32 (OY-TES 1); lymphocyte-specific protein tyrosine kinase (LCK); A kinase anchor protein 4 (AKAP-4); synovial sarcoma, X breakpoint 2 (55X2); Leukocyte-associated immunoglobulin-like receptor 1 (LAIR1); Fc fragment of IgA receptor (FCAR); Leukocyte immunoglobulin-like receptor subfamily A member 2 (LILRA2); CD300 molecule-like family member f (CD300LF); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1).

47. Method according to claim 45, wherein said immune cell engager binds a tumor antigen selected from: CD19, CD20 and/or CD22.

48. Method according to claim 45, wherein said immune cell engager binds a tumor antigen selected from: CD123 and/or CLL1.

49. Method according to claim 45, wherein said immune cell engager binds a tumor antigen selected from: BCMA, CD38 and/or CS1.

50. Method according to claim 45, wherein said immune cell engager binds a solid tumor antigen selected from: Mesothelin, MUC1, ROR1, B7H3, CDH3, CEA, DLL3, EGFR, EpCAM, GD2, GPA33, GPC3, HER2, PSCA, PSMA, SSTR2.

51. Method according to any one of claims 1 to 50, wherein said method further comprises expressing a chimeric antigen receptor into said T-cells.

52. Method according to claim 51, wherein said CAR is directed against a tumor antigen selected from Interleukin 3 receptor subunit alpha.spanning 4-domains A1 (MS4A1 also known as CD20); CD22 molecule (CD22); CD229 molecule (CD229) CD24 molecule (CD24); CD248 molecule (CD248); CD276 molecule (CD276 or B7H3); CD3 molecule (CD3); CD33 molecule (CD33); CD38 molecule (CD38); CD44v6; CD5 molecule (CD5); CD56 molecule (CD56); CD7 molecule (CD7); CD70 molecule (CD70); CD72; CD79a; CD79b; TNF receptor superfamily member 8 (TNFRSF8 also known as CD30); KIT proto-oncogene receptor tyrosine kinase (CD117); V-set pre-B cell surrogate light chain 1 (VPREB1 or CD179a); adhesion G protein-coupled receptor E5 (ADGRE5 or CD97); TNF receptor superfamily member 17 (TNFRSF17 also known as BCMA); SLAM family member 7 (SLAMF7 also known as CS1); L1 cell adhesion molecule (L1CAM); C-type lectin domain family 12 member A (CLEC12A also known as CLL-1); tumor-specific variant of the epidermal growth factor receptor (EGFRvIII); thyroid stimulating hormone receptor (TSHR); Fms related tyrosine kinase 3 (FLT3); ganglioside GD3 (GD3); Tn antigen (Tn Ag); lymphocyte antigen 6 family member G6D (LY6G6D); Delta like canonical Notch ligand 3 (DLL3); Interleukin-13 receptor subunit alpha-2 (IL-13RA2); Interleukin 11 receptor subunit alpha (IL11RA); mesothelin (MSLN); Receptor tyrosine kinase like orphan receptor 1 (ROR1); Prostate stem cell antigen (PSCA); erb-b2 receptor tyrosine kinase 2 (ERBB2 or Her2/neu); Protease Serine 21 (PRSS21); Kinase insert domain receptor (KDR also known as VEGFR2); Lewis y antigen (LewisY); Solute carrier family 39 member 6 (SLC39A6); Fibroblast activation protein alpha (FAP); Hsp70 family chaperone (HSP70); Platelet-derived growth factor receptor beta (PDGFR-beta); Cholinergic receptor nicotinic alpha 2 subunit (CHRNA2); Stage-Specific Embryonic Antigen-4 (SSEA-4); Mucin 1, cell surface associated (MUC1); mucin 16, cell surface associated (MUC16); claudin 18 (CLDN18); claudin 6 (CLDN6); Epidermal Growth Factor Receptor (EGFR); Preferentially expressed antigen in melanoma (PRAME); Neural Cell Adhesion Molecule (NCAM); ADAM metallopeptidase domain 10 (ADAM10); Folate receptor 1 (FOLR1); Folate receptor beta (FOLR2); Carbonic Anhydrase IX (CA9); Proteasome subunit beta 9 (PSMB9 or LMP2); Ephrin receptor A2 (EphA2); Tetraspanin 10 (TSPAN10); Fucosyl GM1 (Fuc-GM1); sialyl Lewis adhesion molecule (sLe); TGS5; high molecular weight-melanoma-associated antigen (HMWMAA); o-acetyl-GD2 ganglioside (OAcGD2); tumor endothelial marker 7-related (TEM7R); G protein-coupled receptor class C group 5, member D (GPRC5D); chromosome X open reading frame 61 (CXORF61); ALK receptor tyrosine kinase (ALK); Polysialic acid; Placenta-specific 1 (PLAC1); hexasaccharide portion of globoH glycoceramide (GloboH); NY-BR-1 antigen; uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 family member K (LY6K); olfactory receptor family 51 subfamily E member 2 (OR51E2); TCR Gamma Alternate Reading Frame Protein (TARP); Wilms tumor protein (WT1); ETV6-AML1 fusion protein due to 12; 21 chromosomal translocation (ETV6-AML1); sperm autoantigenic protein 17 (SPA17); X Antigen Family, Member 1E (XAGE1E); TEK receptor tyrosine kinase (Tie2); melanoma cancer testis antigen-1 (MAD-CT-1); melanoma cancer testis antigen-2 (MAD-CT-2); Fos-related antigen 1; p53 mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B 1; v-myc avian myelocytomatosis viral oncogene neuroblastoma derived homolog (MYCN); Ras Homolog Family Member C (RhoC); Cytochrome P450 1B 1 (CYP1B 1); CCCTC-Binding Factor (Zinc Finger Protein)-Like (BORIS); Squamous Cell Carcinoma Antigen Recognized By T Cells 3 (SART3); Paired box protein Pax-5 (PAX5); proacrosin binding protein sp32 (OY-TES 1); lymphocyte-specific protein tyrosine kinase (LCK); A kinase anchor protein 4 (AKAP-4); synovial sarcoma, X breakpoint 2 (55X2); Leukocyte-associated immunoglobulin-like receptor 1 (LAIR1); Fc fragment of IgA receptor (FCAR); Leukocyte immunoglobulin-like receptor subfamily A member 2 (LILRA2); CD300 molecule-like family member f (CD300LF); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1).

53. Method according to claim 51 or 52, wherein said CAR(s) and said immune cell engager(s) are directed against the same antigen.

54. Method according to claim 51 or 52, wherein said CAR(s) and said immune cell engager(s) are directed against distinct tumor associated antigens to mitigate tumor escape and/or on-target/off-target tumor toxicities.

55. Method according to claim 54, wherein said CAR is directed against CD22, while said IC engager is directed against CD20 and/or CD19.

56. Method according to claim 54, wherein said CAR is directed against CS1, while said IC engager is directed against BCMA and/or CD38, or vice-versa.

57. Method according to claim 54, wherein said CAR is directed against CD123, while said IC engager is directed against CLL1 or vice-versa.

58. Method according to claim 54, wherein said CAR is directed against mesothelin, while said IC engager is directed against Trop2 or vice-versa.

59. Method according to any one of claims 51 to 58, wherein the polynucleotide sequence(s) for expression of said CAR and/or said immune cell engager is(are) introduced at a locus regulated by or encoding TCR, HLA, β2m, PD1, CTLA4, TIM3, LAG3, GM-CSF, CD25 and/or CD69.

60. Method according to any one of claims 51 to 58, wherein said CAR and said soluble immune cell engager are co-expressed, preferably co-transcribed, more preferably transcribed from the same polynucleotide while comprising a self-cleaving peptide.

61. Method according to claim 60, wherein said polynucleotide sequence(s) for expression of said CAR and/or said immune cell engager are introduced in the immune cells using unique or separate viral vector(s).

62. An engineered T-cell, the genotype of which is: [TCR]^{negative}[IC engager]^{positive}.

63. An engineered T-cell, the genotype of which is:
[TCR]^{negative}[IC engager]^{positive} [CAR]^{positive} such as disclosed in Table 15

64. An engineered T-cell according to claim 62 or 63, wherein said IC engager is directed to a component of TCR, CD16 or CD40.

65. An engineered T-cell according to claim 64, wherein said component of the T-cell receptor is selected from CD3, TCRalpha, TCRbeta, TCRgamma and/or TCR delta.

66. An engineered T-cell according to any one of claims 62 to 65, wherein said T-cell is a primary cell.

67. An engineered T-cell according to any one of claims 62 to 66, wherein said T-cell is a mammalian cell, preferably a human cell.

68. An engineered T-cell according to any one of claims 62 to 67, wherein at least one allele encoding TCR alpha, TCRbeta, and/or CD3 has been inactivated by mutation.

69. An engineered T-cell according to any one of claims 62 to 68, wherein at least one allele selected from β2m, PD1, CTLA4, GM-CSF, dCK, CD52 and/or GR has been inactivated.

70. An engineered T-cell according to any one of claims 62 to 69, wherein said T-cell comprises an exogenous polynucleotide encoding a soluble immune cell engager directed against immune cells selected from T-cells, NK-cells, macrophages or antigen presenting cells (APC).

71. An engineered T-cell according to claim 70, wherein said soluble immune cell engager binds an immune cell's activating receptor complex.

72. An engineered T-cell according to claim 70 or 71, wherein said soluble immune cell engager binds a component of the TCR, such as CD3, TCRalpha, TCRbeta, TCRgamma and/or TCR delta.

73. An engineered T-cell according to any one of claims 62 to 69, wherein said soluble immune cell engager binds a CD3 antigen.

74. An engineered T-cell according to any one of claims 62 to 69, wherein said cell comprises an exogenous polynucleotide encoding a soluble immune cell engager directed against NK cells.

75. An engineered T-cell according to claim 74, wherein said immune cell engager binds CD16 surface antigen.

76. An engineered T-cell according to any one of claims 62 to 69, wherein said cell comprises an exogenous polynucleotide encoding a soluble immune cell engager directed against APC/macrophages.

77. An engineered T-cell according to claim 76, wherein said soluble immune cell engager binds CD40 surface antigen.

78. An engineered T-cell according to any one of claims 62 to 77, wherein said immune cell engager further specifically binds at least one infection associated antigen or tumor antigen.

79. An engineered T-cell according to claim 78, wherein said immune cell engager binds a tumor antigen selected from: CD19, CD20 and/or CD22.

80. An engineered T-cell according to claim 78, wherein said immune cell engager binds a tumor antigen selected from: CD123 and/or CLL1.

81. An engineered T-cell according to claim 78, wherein said immune cell engager binds a tumor antigen selected from: BCMA, CD38 and/or CS1.

82. An engineered T-cell according to claim 78, wherein said immune cell engager binds a solid tumor antigen selected from: Mesothelin, MUC1, ROR1, B7H3, CDH3, CEA, DLL3, EGFR, EpCAM, GD2, GPA33, GPC3, HER2, PSCA, PSMA, SSTR2.

83. An engineered T-cell according to claim 78, wherein said immune cell engager binds a liquid tumor antigen selected from: CD19, CD20, CD22, CD30, CD33, CD38, CD123, BCMA and FCRH5.

84. An engineered T-cell according to any one of claims 62 to 83, wherein said soluble immune cell engager is selected from bispecific T-cell engagers (BITE), dual-affinity re-targeting antibodies (DART), bispecific engagement by antibodies based on the T-cell receptor (BEAT), CROSSMAB, TRIOMAB, tandem diabody (TANDAB), ADAPTIR, affinity-tailored adaptors for T-cells (ATAC), DUOBODY, XMAB, T-cell redirecting antibody (TRAB), BICLONICS, DUTAMAB, VELOCI-BI, hinge-mutated, bispecific antibody-armed activated t-cells (AATC), bi- & tri-specific killer cell engagers (BIKE, TRIKE).

85. An engineered T-cell according to claim 84, said immune cell engager is a BiTE.

86. An engineered T-cell according to claim 84, wherein said IC engager binds CD3 and CD19.

87. An engineered T-cell according to claim 85, wherein said IC engager is a BiTE having a polypeptide sequence comprising SEQ ID NO. 42.

88. An engineered T-cell according to claim 84, wherein said IC engager binds CD3 and CD22.

89. An engineered T-cell according to claim 84, wherein said IC engager binds CD3 and CD20.

90. An engineered T-cell according to claim 84, wherein said IC engager binds CD3 and CD123.

91. An engineered T-cell according to claim 84, wherein said IC engager binds CD3 and CLL1.

92. An engineered T-cell according to claim 84, wherein said IC engager binds CD3 and Mesothelin.

93. An engineered T-cell according to claim 84, wherein said IC engager binds CD3 and Trop2.

94. An engineered T-cell according to claim 84, wherein said IC engager binds CD16 and CD19.

95. An engineered T-cell according to claim 84, wherein said IC engager binds CD16 and CD22.

96. An engineered T-cell according to claim 84, wherein said IC engager binds CD16 and CD20.

97. An engineered T-cell according to claim 84, wherein said IC engager binds CD16 and CD123.

98. An engineered T-cell according to claim 84, wherein said IC engager binds CD16 and CLL1.

99. An engineered T-cell according to claim 84, wherein said IC engager binds CD16 and Mesothelin.

100. An engineered T-cell according to claim 84, wherein said IC engager binds CD16 and Trop2.

101. An engineered T-cell according to claim 84, wherein said IC engager binds CD40 and CD19.

102. An engineered T-cell according to claim 84, wherein said IC engager binds CD40 and CD22.

103. An engineered T-cell according to claim 84, wherein said IC engager binds CD40 and CD20.

104. An engineered T-cell according to claim 84, wherein said IC engager binds CD40 and CD123.

105. An engineered T-cell according to claim 84, wherein said IC engager binds CD40 and CLL1.

106. An engineered T-cell according to claim 84, wherein said IC engager binds CD40 and Mesothelin.

107. An engineered T-cell according to claim 84, wherein said IC engager binds CD40 and Trop2.

108. An engineered T-cell according to any one of claims 62 to 107, wherein the polynucleotide sequence for expression of said IC engager has been introduced at a locus regulated by or encoding TCR, HLA, β2m, PD1, CTLA4, TIM3, LAG3, GM-CSF, CD69. IL2Ra and/or CD52.

109. An engineered T-cell according to any one of claims 62 to 108, wherein the polynucleotide sequence for expression of said immune cell engager is introduced by using a viral vector.

110. An engineered T-cell according to any one of claims 62 to 109, wherein said CAR is directed against a tumor associated antigen selected from Mesothelin, MUC1, ROR1, B7H3, CDH3, CEA, DLL3, EGFR, EpCAM, GD2, GPA33, GPC3, HER2, PSCA, PSMA, SSTR2, CD19, CD20, CD22, CD30, CD33, CD38, CD123, BCMA and FCRH5.

111. An engineered T-cell according to claim 109 or 110, wherein said CAR(s) and said immune cell engager(s) are directed against the same antigen.

112. An engineered T-cell according to claim 109 or 110, wherein said CAR(s) and said immune cell engager(s) are directed against distinct tumor associated antigens to mitigate tumor escape and/or on-target/off-target tumor toxicities.

113. An engineered T-cell according to claim 112, wherein said CAR is directed against CD22, while said IC engager is directed against CD20 and/or CD19.

114. An engineered T-cell according to claim 112, wherein said CAR is directed against CS1, while said IC engager is directed against BCMA and/or CD38, or vice-versa.

115. An engineered T-cell according to claim 112, wherein said CAR is directed against CD123, while said IC engager is directed against CLL1 or vice-versa.

116. An engineered T-cell according to claim 112, wherein said CAR is directed against mesothelin, while said IC engager is directed against Trop2 or vice-versa.

117. An engineered T-cell according to any one of claims 112 to 116, wherein the polynucleotide sequence(s) for expression of said CAR and/or said immune cell engager is(are) introduced at a locus regulated by or encoding TCR, HLA, β2m, PD1, CTLA4, TIM3, LAG3, GM-SCF, CD25 and/or CD69.

118. An engineered T-cell according to any one of claims 112 to 117, wherein said CAR and said soluble immune cell engager are co-expressed, preferably co-transcribed, more preferably transcribed from the same polynucleotide while comprising a self-cleaving peptide.

119. An engineered T-cell according to any one of claims 112 to 117, wherein said polynucleotide sequence(s) for expression of said CAR and/or said immune cell engager are introduced in the immune cells using unique or separate viral vector(s).

120. A population of genetically engineered immune cells, in which expression of TCR is repressed or inactivated, characterized in that at least 2%, 5%, 10%, 20%, 40%, 50%, 60%, 70%, 80%, 90%, 95% or 99% of said cells exogenously express a soluble immune cell engager.

121. A population of genetically engineered immune cells obtainable by a method according to any one of claims 1 to 61.

122. A population of genetically engineered immune cells comprising at least 1%, preferably at least 2%, 5%, 10%, 20%, 40%, 50%, 60%, 70%, 80%, 90%, 95% or 99% of engineered T-cells according to any one of claims 62 to 119.

123. A population of genetically engineered immune cells according to any one of claims 120 to 122, for use as a medicament.

124. A population of genetically engineered immune cells according to any one of claims 120 to 122, for allogeneic use in a method of treating a cancer or infection.

125. A population of genetically engineered immune cells according to any one of claims 120 to 122, for use prior to stem cells transplantation (“bridge to transplant”).

126. A therapeutic composition comprising at least 2%, 5%, 10%, 20%, 40%, 50%, 60%, 70%, 80%, 90%, 95% or 99% of genetically engineered T-cells according to any one of claims 61 to 119.

127. A therapeutic composition comprising a population of genetically engineered immune cells according to any one of claims 120 to 125.

128. A method of treating a patient who has a cancer or infection correlated with a specific antigen marker at the malignant or infected cell surface, said method comprising the administration to said patient of genetically engineered allogeneic T-cells, wherein TCR expression is reduced or suppressed, wherein said engineered T-cells express soluble immune cell engager(s) that directs patient's immune cells against malignant or infected cells.

129. A method of treating a patient who has a cancer or infection correlated with a specific antigen marker at the malignant or infected cell surface, said method comprising the administration to said patient of a population of genetically engineered T-cells according to any one of claims 120 to 125.

130. The method according to any one of claim 128 or 129, wherein said genetically engineered T-cells co-express (1) a CAR directed against said antigen marker at the malignant or infected cell surface, and (2) a soluble immune cell engager that selectively binds immune cells.

131. The method according to any one of claim 128 or 129, wherein said immune cell engager redirects TCR positive cells, preferably the patient's immune cells, against said malignant or infected cells.

132. The method according to any one of claim 128 or 129, wherein said method comprises a preliminary step of lymphodepletion of the patient's immune cells.

133. The method according to claim 132, wherein said lymphodepletion is performed with an agent targeting CD52, such as alemtuzumab.

134. The method according to claim 133, wherein said genetically engineered T-cells have a CD52 gene repressed or inactivated.

135. The method according to any one of claim 128 or 129, wherein said soluble immune cell engager allows the elimination of TCR positive cells in the population of genetically engineered T-cells and/or among the patient's immune cells.

136. The method according to any one of claim 128 or 129, wherein the genetically engineered T-cells originate from at least one donor.

137. The method according to any one of claim 128 or 129, wherein said method comprises a further step of bone marrow stem cells transplantation.

138. The method according to any one of claim 128 or 129, wherein said soluble immune cell engager produced by the genetically engineered T-cells reduces fratricide killing of said genetically engineered immune cells by the patient's immune cells.

139. Method for increasing the persistence of allogeneic TCR deficient CAR positive T-cells into a patient, wherein said method comprises the step of expressing in said allogeneic CAR T-cells a soluble immune cell engager, wherein said immune cell engager binds the immune cells produced by the patient's immune system.

140. Method according to claim 139, wherein said soluble immune cell engager protects said allogeneic CAR T-cells from the killing activity by the patient's immune cells.

141. Method according to claim 139, wherein said soluble immune cell engager redirects the patient's immune cells against malignant or infected cells.

142. Method for increasing the number of gamma/delta T-cells active against malignant or infected cells in a patient after lymphodepletion, wherein said method comprises administering allogeneic TCR deficient T-cells, wherein said T-cells express soluble T-cell engager(s) that specifically binds (1) TCR gamma delta cells and (2) at least one specific antigen marker at the surface of said malignant or infected cells.

143. Method for enriching a population of T-cells in which the expression of TCR is reduced or inactivated, wherein genetically engineered immune cells from a population according to any one of claims 120 to 122 are cultured together in condition where at least one soluble T-cell engager is expressed, so as to eliminate the TCR positive cells from said population.
